(19) United States
(12) Patent Application Publication
(10) Pub. No.: US 2024/0257662 A1
(43) Pub. Date: Aug. 1, 2024

(54) DIGITAL APPARATUS AND APPLICATION FOR CANCER CACHEXIA THERAPY AND METHODS OF USE THEREOF

(71) Applicant: S-Alpha Therapeutics, Inc., Seoul (KR)

(72) Inventor: Seung Eun Choi, Seoul (KR)

(73) Assignee: S-Alpha Therapeutics, Inc., Seoul (KR)

(21) Appl. No.: 18/423,265

(22) Filed: Jan. 25, 2024

Related U.S. Application Data
(63) Continuation of application No. 18/082,722, filed on Dec. 16, 2022, now Pat. No. 11,903,721.
Application No. 18/082,722 is a continuation of application No. 16/860,641, filed on Apr. 28, 2020, now abandoned.

(30) Foreign Application Priority Data
Dec. 31, 2019 (KR) 10-2019-0180050

(51) Int. Cl. G09B 19/00; A61B 5/00; G09B 5/02

(52) U.S. Cl. CPC G09B 19/00; A61B 5/4088; A61B 5/7465; G09B 5/02

(57) ABSTRACT

Systems and methods for cancer cachexia therapy are provided. A system may include a digital apparatus, which may include a digital instruction generation unit configured to generate digital therapeutic modules for treating cancer cachexia, generate specified digital instructions based on the digital therapeutic modules and provide the digital instructions to a first user, and an outcome collection unit configured to collect the first user's execution outcomes of the digital instructions.

### BACKGROUND

Dementia refers to a clinical syndrome that disables a person's ability to perform everyday activities due to postnatal cognitive decline in memories, languages, judgments, etc. Types of dementia are divided into degenerative dementia which includes Alzheimer's disease (AD), vascular dementia caused due to stroke, and others caused due to various factors such as injuries or drugs. Currently, U.S. Food and Drug Administration (FDA) approves two types of drugs for AD treatment: cholinesterase inhibitors (e.g., Donepezil, Rivastigmine and Galantamine) and N-methyl-D-aspartate (NMDA) antagonist (e.g., Memantine). However, the therapeutic effect of these drugs is highly limited. Specifically, the aforementioned drugs only serve to ease the symptoms rather than halt disease's progression or cure the disease itself. In addition, the questions of the uselessness of drug treatment for dementia have been raised as drug treatment often entails serious fatal side effects. Furthermore, the fact that there are too many interactions between drugs may be dangerous to patients who take such drugs. Thus, methods and apparatuses that can treat or inhibit the progression of amnestic MCI and AD without such limitations are needed. Cancer cachexia is characterized by an ongoing loss of skeletal muscle mass (with or without loss of fat mass) that cannot be fully reversed by conventional nutrition. Current therapies for cachexia include medication aimed at retarding or halting progression of the disorder. Treatments include, for example, orexigenic agents (i.e., appetite stimulants), corticosteroids, cannabinoids, serotonin antagonists, prokinetic agents, androgens and anabolic agents, anticytokine agents, non-steroidal anti-inflammatory drugs, and regulators of circadian rhythm, with most therapies directed to treating the underlying or associated condition (e.g., cancer). However, such treatment (e.g., using small molecules, biologics, etc.) is often compromised by the patient's inability to tolerate the treatment due to their cachexia. Thus, there is a need in the art for improved treatments (e.g., digital therapeutics) for wasting disorders, such as cachexia.

### SUMMARY

Methods and apparatuses are described herein for treating a patient with mild cognitive impairment (MCI) or dementia by one or more digital therapeutics. For example, a medical profession (e.g., a doctor) may determine whether the patient has the MCI or the dementia based on one or more symptoms of the MCI or the dementia. If the patient has been diagnosed with the MCI or the dementia, the medical professional may prescribe and/or administer the one or more digital therapeutics to the patient to improve a plurality of neurohumoral factors that cause the MCI or the dementia. The one or more digital therapeutics comprise one or more digital instructions that are generated to treat at least one imbalance of the plurality of neurohumoral factors based on at least one neurohumoral change among the plurality of neurohumoral factors by the patient's performance of the one or more digital instructions. The plurality of neurohumoral factors may include at least one of sex steroid hormone, insulin-like growth factor-2 (IGF-2), β-catenin in Wnt signaling, Bcl-2-associated athanogene 1 (BAG1), cAMP response element-binding protein (CREB), inflammation factors, corticosteroids, or neurohormones. The at least one imbalance of the plurality of neurohumoral factors includes at least one of a sex steroid hormone imbalance, a IGF-2 decrease, a β-catenin degradation, a BAG1 inactivation, a CREB inactivation, an increase in inflammation factors, a corticosteroids increase, or a neurohormone decrease. The one or more digital therapeutics is performed by a user's device. The present disclosure relates to digital therapeutics (hereinafter referred to as DTx) intended for cancer cachexia therapy. The present disclosure also relates to systems that integrate digital therapeutics with one or both of a healthcare provider portal and an administrative portal to treat cancer cachexia in a patient. Some embodiments of the present disclosure may comprise deducing a mechanism of action (hereinafter referred to as MOA) in cancer cachexia, and establishing a therapeutic hypothesis and a digital therapeutic hypothesis for inhibiting progression of cancer cachexia, and treating the cancer cachexia based on these findings.

### BRIEF DESCRIPTION OF THE DRAWINGS

A more detailed understanding may be had from the following description, given by way of example in conjunction with the accompanying drawings, wherein like reference numerals in the figures indicate like elements, and wherein:

FIG. 1A is a system diagram illustrating an example mechanism of action (MOA) of Alzheimer's disease (AD);

FIG. 1B is a system diagram illustrating an example treatment procedure based on therapeutic hypothesis for treating or inhibiting progression of MCI and AD;

FIG. 1C is a system diagram illustrating another example treatment procedure based on a user device for treating or inhibiting progression of MCI and AD;

FIG. 2 is a diagram illustrating an example device that can be used to treat MCI and AD;

FIG. 3 is a diagram illustrating an example input and output loop of a user device for treating or inhibiting progression of MCI and AD;

FIG. 4 is a diagram illustrating an example feedback loop for treating or inhibiting progression of and treating MCI and AD;

FIG. 5A is a diagram illustrating example modules to implement digital therapeutics for treating or inhibiting progression of MCI and AD;

FIG. 5B is a diagram illustrating example background factors that support a user device for treating or inhibiting progression of MCI and AD;

FIG. 6A is a diagram illustrating an example method of assigning a patient-customized prescription;

FIG. 6B is a diagram illustrating an example method of assigning a patient-customized digital prescription;

FIG. 7A is a diagram illustrating an example digital instruction for an execution environment setting;

FIG. 7B is a diagram illustrating an example digital instruction for a lifestyle module;

FIG. 7C is a diagram illustrating an example digital instruction for a memory/learning module;

FIG. 7D is a diagram illustrating an example digital instruction for an exercise module;

FIG. 7E is a diagram illustrating an example digital instruction for a positive/achievement module;

FIG. 8 is a diagram illustrating an example procedure using an digital application for treating or inhibiting progression of MCI and AD;

FIG. 9 is a diagram illustrating an example procedure for generating digital instructions to treat or inhibit progression of MCI and AD;

FIG. 10 is a diagram illustrating an example procedure that repeats the execution of digital instructions based on feedback for treating or inhibiting progression of MCI and AD;

FIG. 11 is a diagram illustrating another example procedure that repeats the execution of digital instructions based on feedback for treating or inhibiting progression of MCI and AD;

FIG. 12 is a diagram illustrating an example procedure for treating a patient with MCI or dementia by digital therapeutics; and

FIG. 13 is a diagram illustrating an example hardware configuration of a user device for treating or inhibiting progression of MCI and AD.

FIG. 14 illustrates muscle atrophy associated with cancer cachexia;

FIG. 15 illustrates various biochemical pathways and physiological symptoms associated with cancer cachexia, as well as applications of certain embodiments of the present disclosure and associated effects;

FIG. 16 is a block diagram showing an exemplary configuration of a digital apparatus for treating cancer cachexia according to one embodiment of the present disclosure;

FIG. 17 is a diagram showing exemplary input and output loops of a digital application for treating cancer cachexia according to one embodiment of the present disclosure;

FIG. 18 is a diagram showing an exemplary background factors supporting the digital apparatus and the application for treating cancer cachexia according to one embodiment of the present disclosure;

FIG. 19 depicts diagrams showing an exemplary method of assigning a patient-customized digital prescription using the digital apparatus and the application for treating cancer cachexia according to one embodiment of the present disclosure;

FIG. 20 is a flowchart illustrating exemplary operations in a digital application for treating cancer cachexia according to one embodiment of the present disclosure;

FIG. 21 is a diagram showing an exemplary hardware configuration of the digital apparatus for treating cancer cachexia according to one embodiment of the present disclosure;

FIG. 22 is a flow chart illustrating an exemplary system for treating cancer cachexia, the system comprising an administrative portal (e.g., Administrator's web), a healthcare provider portal (e.g., Doctor's web) and a digital apparatus configured to execute a digital application (e.g., an application or ‘app’) for treating cancer cachexia in a subject;

FIG. 23 is a flow chart illustrating an exemplary digital application usage flow of the present disclosure;

FIG. 24 is a flow chart illustrating an exemplary execution flow for a login verification during a splash process at the starting of a digital application of the present disclosure;

FIG. 25 is a diagram illustrating an exemplary patient portal structure of a digital application of the present disclosure;

FIG. 26 is a flow chart illustrating an exemplary patient portal of internal activity use flow of a digital application of the present disclosure;

FIG. 27 is a flow chart illustrating an exemplary patient portal structure of the present disclosure;

FIG. 28 is a flow chart illustrating an exemplary usage flow of a digital application of the present disclosure;

FIG. 29 is a flow chart illustrating an exemplary target heart rate calculation formula of a digital application of the present disclosure;

FIG. 30 is a diagram illustrating an exemplary scheduled exercise sessions of a digital application of the present disclosure;

FIG. 31 is a diagram illustrating an exemplary patient portal structure of a digital application of the present disclosure;

FIG. 32 is a flow chart illustrating an exemplary doctor portal structure and administrative portal structure of a digital application of the present disclosure;

FIG. 33 is a flow chart illustrating an exemplary execution flow for an administrative portal in a system of the present disclosure;

FIG. 34 is a diagram illustrating an exemplary usage flow of a digital application of the present disclosure;

FIG. 35 is a diagram illustrating an exemplary screen touch sensing configuration for the voluntary skeletal muscle exercise module;

FIG. 36 is a diagram illustrating an exemplary usage flow of a digital application of the present disclosure;

FIG. 37 is a diagram illustrating an exemplary head lifting sensing configuration for the voluntary skeletal muscle exercise module;

FIG. 38 is a diagram illustrating an exemplary head turning sensing configuration for the voluntary skeletal muscle exercise module;

FIG. 39 is a flow chart illustrating an exemplary execution flow for the head turning instructions for the voluntary muscle exercise module;

FIG. 40 is a diagram illustrating an exemplary usage flow of a digital application of the present disclosure;

FIG. 41 is a flow chart illustrating an exemplary execution flow for the hand gripping instructions for the voluntary muscle exercise module;

FIG. 42 is a diagram illustrating an exemplary usage flow of a digital application of the present disclosure;

FIG. 43 is a diagram illustrating an exemplary arm shaking configuration for the voluntary skeletal muscle exercise module;

FIG. 44 is a diagram illustrating an exemplary leg lifting configuration for the voluntary skeletal muscle exercise module;

FIG. 45 is a flow chart illustrating an exemplary execution flow for the leg lifting instructions for the voluntary muscle exercise module.

### DETAILED DESCRIPTION

According to “Year 2016 nationwide dementia epidemiology survey” (Report Number: NDR-1603-0015, 2017, June, Central Dementia Center, Ministry of Health and Welfare, Korea, Republic of.), the standardized dementia prevalence rate of male Korean elderly among aged 65 or older was 8.18% and that of the female was 10.46%, making a total of 9.50%. The data shows that women's prevalence rate was higher than that of men.

Further specifying the types of dementia from the standardized dementia prevalence rate among Korean elderly aged 65 or older (9.50%), AD counted for 7.07% of the total, while vascular dementia counted for 0.83% and the others for 1.60%. It is clear that AD takes an overwhelmingly large proportion (approximately ¾) when compared to the other types of dementia.

What is noticeable is the increase in the standardized dementia prevalence rate along with population ageing. Comparing the standardized prevalence rate of Korean elderly aged 65 or older with that of the Korean elderly aged 85 or older, the rate goes up from 9.50% to 38.39%. The standardized dementia prevalence rate of men aged 85 or older, in particular, rises to 53.99%.

The dementia prevalence rate of Korean elderly and the number of dementia patients estimated with the future population based on the data concerning age, sex and region standardization of 2015 population census is even more noticeable. The standardized dementia prevalence rate among those aged 65 or older in 2016 of 9.73% is estimated to rise as 10.29% in 2020, 10.56% in 2030, and 16.09% in 2050. According to the estimated shift, the number of Korean dementia patients by 2025 is over 1 million, and by 2050 over 3 million.

MCI (Korean Standard Disease and Sign Classification Code: F06.7), as an intermediate state between normal aging and dementia, does not cause any difficulty in everyday activities but results in a decline of memories and cognitive functions compared to a person's age group. Types of MCI are divided into amnestic MCI which entails memory loss and non-amnestic MCI which causes a decline of non-memory-related cognitive functions. As it is known that about half of MCI develops into dementia, MCI, in terms of preventing dementia, is receiving attention from the public.

According to the aforementioned report, the standardized MCI prevalence rate for male Korean elderly aged 65 or older is 18.09%, and that of the female was 25.28%, making a total of 22.25%. The data shows that, as have been observed from the dementia prevalence rate, the female prevalence rate among those aged 65 or older was higher than that of men.

Further specifying the types of MCI from the standardized MCI prevalence rate among Korean elderly aged 65 or older (22.25%), amnestic MCI counted for 16.69% while non-amnestic MCI counted for 5.56%. Amnestic MCI takes about ¾ of the total proportion. Amnestic MCI particularly requires a more active care as it has higher risks to be developed as dementia although it may not cause any difficulty in everyday lives.

Concerning the increase in the prevalence rate, dementia care cost per patient and government budget on dementia care should be noted. According to Korean Dementia Observatory 2018, Central Dementia Center, dementia care cost per patient is 20.74 million KRW and the government budget allocated for dementia care is 14.6 trillion KRW, which accounts for 0.8% of national GDP. Social costs of dementia is estimated to increase sharply from total investment 17.9 trillion KRW in 2020 to 87.2 trillion KRW in 2050, Korea, Republic of.

The increase in both the dementia prevalence rate and the government budget on dementia care is a global issue that many countries encounter although the specifics may vary depending on regions and progressions of population ageing.

In case of a vascular dementia patient, he or she may be cured by performing surgeries if the cause of dementia is due to stroke, brain tumor or normal pressure hydrocephalus (NPH). If it is the case of vascular dementia caused due to brain infraction, the patient may prevent the disease or delay its progression by eliminating or managing elements such as hypertension, diabetes, smoking, hyperlipidemia, etc. However, it is hard to expect a positive therapeutic effect with the aforementioned methods from an AD, which is degenerative dementia, patient unlike those with vascular dementia.

Currently, US FDA approves two types of drugs for AD treatment: cholinesterase inhibitors such as Donepezil, Rivastigmine and Galantamine, and N-methyl-D-aspartate (NMDA) antagonist such as Memantine. Donepezil, which is cholinesterase inhibitor, is generally prescribed to treat all stages of AD, and Rivastigmine and Galantamine to treat mild to moderate stage of AD. Memantine in combination with Donepezil is prescribed to treat moderate to severe stage of AD. However, the therapeutic effect of such drug treatment is highly limited. The aforementioned drugs only serve to ease the symptoms rather than halt disease's progression or cure the disease itself. At the same time, the questions of the uselessness of drug treatment for dementia have been raised as drug treatment entails serious and often fatal side effects and the fact that too many interactions between drugs may be dangerous has been pointed out. In fact, in France, insurance plans have suspended to cover the aforementioned four types of dementia treatment since August 2018.

In addition, various cases where multinational pharmaceutical companies fail to develop or stop developing cure for AD (e.g., Fizer's Bapineuzumab, Eli Lilly and Company's Solanezumab, Merck's Verubecestat which is β-secretase (BACE) inhibitor, Boehringer Ingelheim's ‘BI 409306’, Astrazeneca's Saracatinib, Biogen's Aducanumab, Novartis and Amgen's ‘CNP520 (Umibecestat)’, Roche's Crenezumab) simply demonstrate the difficulty of developing AD treatment. Hence, it is predicted that no innovative AD treatment will emerge in near future.

In this disclosure, a digital apparatus and an application for inhibiting progression of and treating amnestic MCI and AD are provided based on mechanism of action (MOA) of amnestic MCI and AD, therapeutic hypothesis and digital therapeutic hypothesis for treating and/or inhibiting progression of MCI and AD.

Embodiments disclosed herein may be based on a rational design of the application in order for clinically verifying the digital therapeutic hypothesis for amnestic MCI and AD and embodying the digital therapy.

The MOA, therapeutic hypothesis and digital therapeutic hypothesis may be deduced based on neuro-humoral factors of amnestic MCI and AD. Based on the digital therapeutic hypothesis for amnestic MCI and AD, a credible digital apparatus and an application which inhibits progression of amnestic MCI and AD and offers improved therapeutic effect through patients' repetitive execution of digital instructions may be provided.

The digital apparatus for MCI and AD treatment in accordance with one embodiment may include a processor generating digital instructions. For example the processor generates a DTx module for MCI and AD treatment bases upon the MOA and therapeutic hypothesis of MCI and AD. The processor may further generate specified digital instructions based on the DTx module, and provide the aforementioned instructions to a first user, and the first user's execution outcomes of the digital instructions may be collected using the apparatus.

The digital application for MCI and AD treatment in accordance with one embodiment, as a digital application stored in a computer-readable medium, may instruct a computing apparatus to execute operations, which comprises: generating a DTx module for treating MCI and AD based on the MOA and therapeutic hypothesis of MCI and AD; generating specified digital instructions based on the DTx module; providing the digital instructions to a first user; and collecting the first user's execution outcomes of the digital instructions.

As described above, MOA, therapeutic hypothesis and digital therapeutic hypothesis may be obtained based on neuro-humoral factors of the progression of amnestic MCI and AD. Patients may be given digital tasks based on these findings, and their execution and completion of tasks may be collected and analyzed in order to effectively inhibit the progression of amnestic MCI and AD and offer improved therapeutic effect.

The development of new drugs starts with confirming a medical demand in situ, proposing a MOA based on the expert reviews and meta-analysis on the corresponding disease, and deducing therapeutic hypothesis based on the expert reviews and the meta-analysis. Also, after a library of drugs whose therapeutic effects are expected is prepared based on the therapeutic hypothesis, a candidate material is found through screening, and the corresponding candidate material is subjected to optimization and preclinical trials to check its effectiveness and safety from a preclinical stage, thereby deciding the candidate material as a final candidate drug. To mass-produce the corresponding candidate drug, a chemistry, manufacturing, and control (CMC) process is also established, a clinical trial is carried out on the corresponding candidate drug to verify a MOA and therapeutic hypothesis of the candidate drug, thereby ensuring the clinical effectiveness and safety of the candidate drug.

Drug targeting and signaling, which fall upstream of the development of new drugs, have many uncertainties. In many cases, because the drug targeting and signaling take a methodology of putting together the outcomes, which have been reported in the art, and interpreting the outcomes, it may be difficult to guarantee the novelty of invention. On the contrary, the invention of drugs capable of regulating the drug targeting and signaling to treat a disease requires the highest level of creativity except for the field of some antibody or nucleic acid (DNA, RNA) therapeutics in spite of the development of research methodology for research and development of numerous new drugs. As a result, the molecular structures of the drugs are the most critical factors in the field of new drugs.

Unlike the drugs, DTx are basically realized using a device that implements DTx. Due to the nature of the DTx, the rational design of DTx against the corresponding disease, and the realization of the DTx based on the rational design may be considered to be a very creative process when considering the clinical verification and approval processes as the therapeutics. That is, the core of the DTx depends on the rational design of DTx suitable for treatment of the corresponding disease, and the development of specific procedures capable of clinically verifying the DTx based on the rational design.

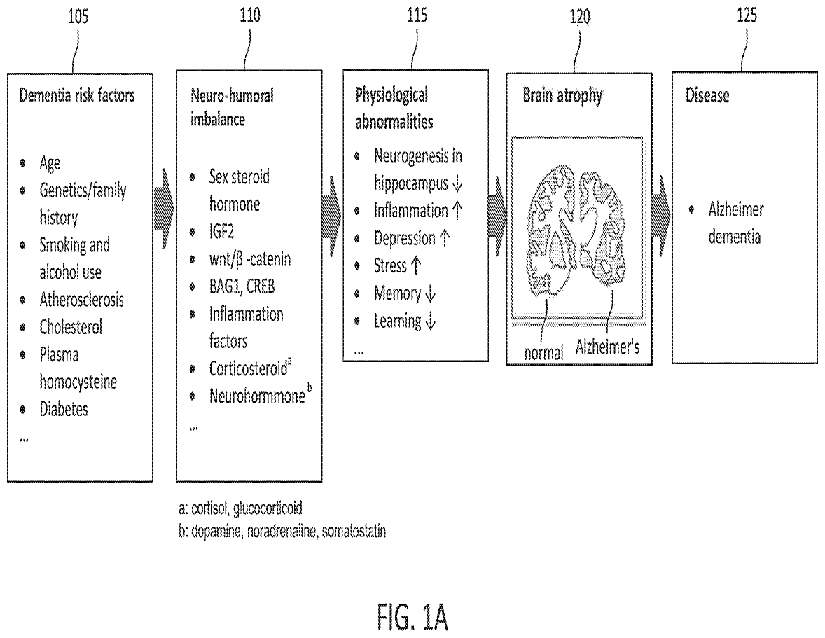

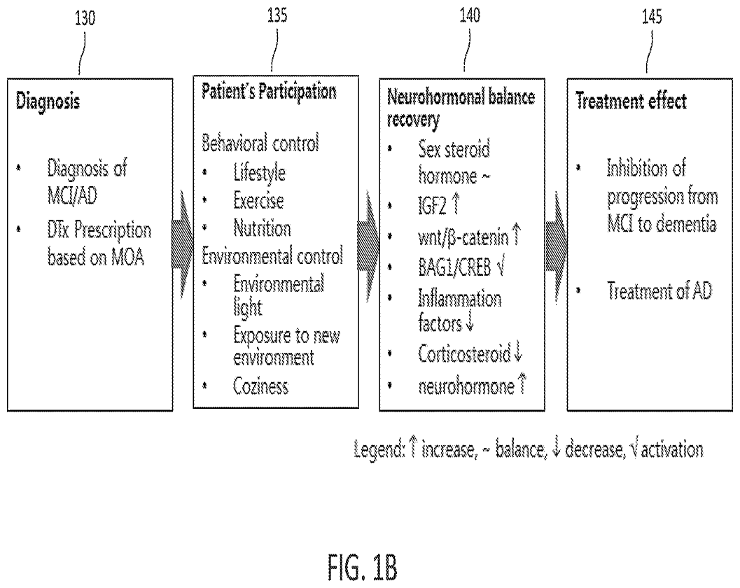

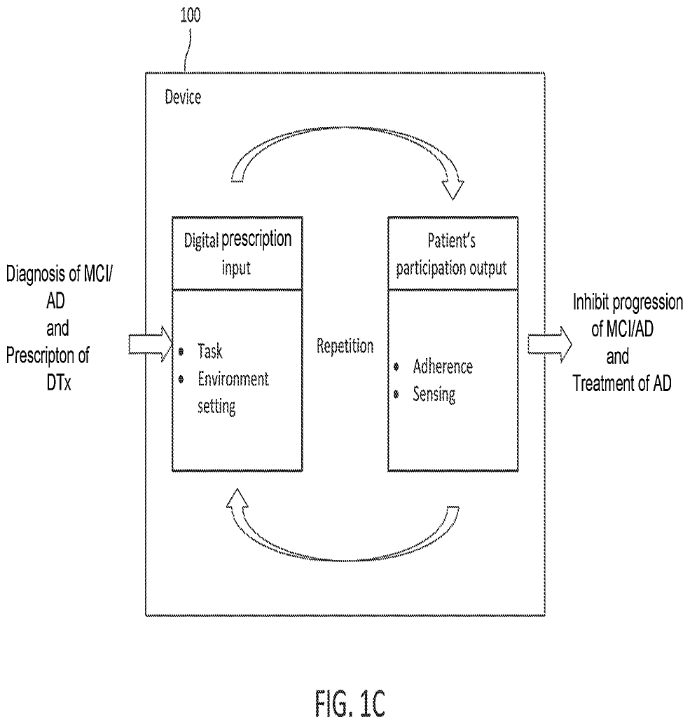

FIG. 1A illustrates an example mechanism of action (MOA) of Alzheimer's disease (AD), which may be used in combination with any of other embodiments described herein. FIG. 1B illustrates an example treatment procedure based on therapeutic hypothesis for treating or inhibiting progression of MCI and AD, which may be used in combination with any of other embodiments described herein. FIG. 1C illustrates another example treatment procedure based on a user device for treating or inhibiting progression of MCI and AD, which may be used in combination with any of other embodiments described herein.

A digital apparatus and an application for inhibiting progression of and treating MCI and AD may be realized based on the MOA and therapeutic hypothesis deduced through the literature search and expert reviews of clinical trial articles on amnestic MCI and AD.

Generally speaking, disease therapy is carried out by analyzing a certain disease in terms of pathophysiological functions and dispositions in order to determine a start point, a progression point, and an end point for the disease. Also, an indication of the disease is defined by characterization of the corresponding disease and statistical analysis of the disease. Also, patient's physiological factors, especially neuro-humoral factors, which correspond to the verified indications, are analyzed, and the patient's neuro-humoral factors are restricted to a narrow extent associated with the disease to deduce a MOA.

Next, therapeutic hypothesis, in which the corresponding disease is treated by controlling actions and environments directly associated with regulation of the corresponding neuro-humoral factors associated with the disease, is deduced. To realize this therapeutic hypothesis into digital therapeutics, digital therapeutic hypothesis for achieving a therapeutic effect through repeated digital instruction and execution, which are associated with the “control of patient's action/environment regulation of neurohumoral factors, is proposed. The digital therapeutic hypothesis can be realized as a digital apparatus and an application configured to present changes in patient's actions (including behavioral, emotional, and cognitive areas), improvement of patient's environment, and patient's participation in the form of specific instructions and collect and analyze execution of the specific instructions.

The literature search for the clinical trials as described above may be executed through meta-analysis and data mining, and the clinical specialist's feedbacks and deep reviews may be applied in each analysis step. Basically, embodiments described herein encompasses extracting a MOA and therapeutic hypothesis for MCI and AD using the procedure as described above, and regulating the neuro-humoral factors based on these results to provide a digital apparatus and an application as DTx for inhibiting progression of and treating MCI and AD.

However, a method of extracting a MOA and therapeutic hypothesis for MCI and AD is not limited to the methods as described above, and MOAs and therapeutic hypotheses for diseases may be extracted using various methods.

Referring to FIG. 1A, various risk factors 105 in aging process such as age, inheritance/family history, smoking & drinking, arteriosclerosis, cholesterol, serum homocysteine, diabetes, MCI and other factors may cause the imbalance of neurohumoral factors in the aging process. For example, neuro-humoral factors 110, which may include abnormality in sex steroid hormone, decline in insulin-like growth factor-2 (IGF-2), abnormality in wnt/β-catenin, decline in BAG1, cAMP response element-binding protein (CREB), hypersecretion of corticosteroids such as inflammation factors, cortisol and glucocorticoid, and hyposecretion of neurohormones such as dopamine, noradrenaline and somatostatin, inhibit neurogenesis in hippocampus in terms of physiological functions, may cause inflammation in brain tissue, and can also cause stress and depression. These physiological abnormalities 115 or physiological factors, over a long period of time, may induce plaque deposits, and neuronal apoptosis, leading to brain atrophy 120 with plaque deposits as a result, which is the anatomical characteristics of AD. As a result, a clinical syndrome or disease 125 commonly referred to as dementia occurs, which entails malfunctioning of brain, decline in cognitive functions such as memory, language and judgment, and hence the difficulties in performing everyday life activities.

Referring to FIG. 1B, the therapeutic hypothesis for MCI and AD relates to inhibition of progression of and treatment of MCI and AD by restoring the balance of neuro-humoral factors 140 through patients' participation 135 including the patients' behavioral control (e.g., lifestyle, habits, exercise, sand nutrition) and environmental control (e.g., bright, familiar, relaxing execution atmosphere setting) after diagnosis 130 of MCI and/or AD. Restoring the balance of neuro-humoral factors 140 may result in treatment effect 145 in MCI and/or AD.

Referring to FIG. 1C, the digital therapeutic hypothesis for MCI and AD may be realized by a device 100 configured to present changes in patients' actions, improvement of patients' environment, and patients' participation in the form of specific instructions, and collect/analyze execution of the specific instructions. When the DTx are used, the imbalance of neuro-humoral factors for amnestic MCI and AD patients may be corrected through the digital inputs (instructions) and outputs (execution) to achieve inhibition of progression of and treatment of MCI and AD.

The MOA and the therapeutic hypothesis for MCI and AD described with reference to FIGS. 1A and 1B are not limited thereto. The methodology may be applied to other types of MCI and AD.

Also, although sex steroid hormone, IGF-2, wnt/β-catenin, BAG1, CREB, inflammation factors, corticosteroids and neurohormones are described as the neuro-humoral factors as shown in FIGS. 1A and 1B, it should be noted that the description of the neuro-humoral factors is given by way of illustration only, and are not intended to be limiting in all aspects of the MOA and the therapeutic hypothesis for amnestic MCI and AD.

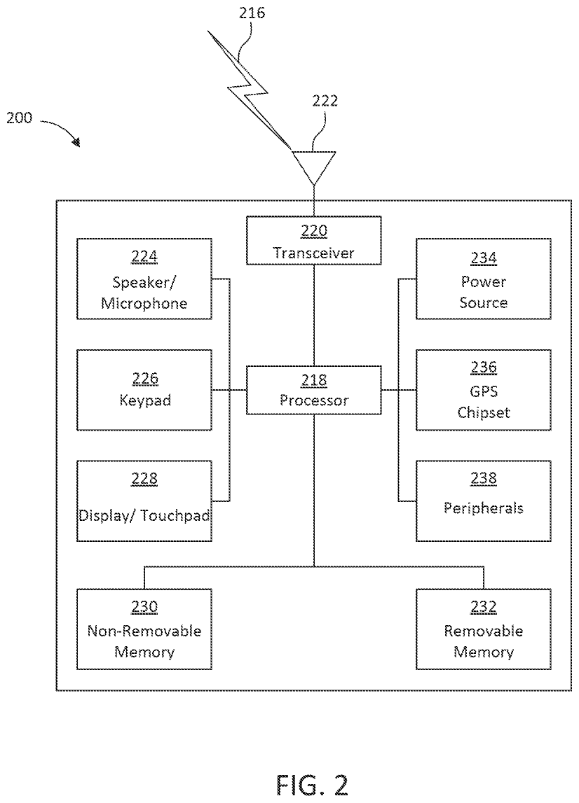

FIG. 2 is a system diagram illustrating an example device 200 that can be used for inhibiting progression of and treating amnestic MCI and AD, which may be used in combination with any of other embodiments described herein. As shown in FIG. 2, the device 200 may include a processor 218, a transceiver 220, a transmit/receive element 222, a speaker/microphone 224, a keypad 226, a display/touchpad 228, non-removable memory 230, removable memory 232, a power source 234, a global positioning system (GPS) chipset 236, and/or other peripherals 238, among others. It will be appreciated that the device 200 may include any sub-combination of the foregoing elements while remaining consistent with an embodiment. By way of example, the device 200 may include a mobile device, a user equipment (UE), a mobile station, a fixed or mobile subscriber unit, a subscription-based unit, a pager, a cellular telephone, a personal digital assistant (PDA), a smartphone, a laptop, a netbook, a personal computer, a wireless sensor, a hotspot or Mi-Fi device, an Internet of Things (IoT) device, a watch or other wearable, a head-mounted display (HMD), a vehicle, a drone, a medical device and applications (e.g., remote surgery), an industrial device and applications (e.g., a robot and/or other wireless devices operating in an industrial and/or an automated processing chain contexts), a consumer electronics device, a device operating on commercial and/or industrial wireless networks, and the like.

The processor 218 may be a general purpose processor, a special purpose processor, a conventional processor, a digital signal processor (DSP), a plurality of microprocessors, one or more microprocessors in association with a DSP core, a controller, a microcontroller, Application Specific Integrated Circuits (ASICs), Field Programmable Gate Arrays (FPGAs), any other type of integrated circuit (IC), a state machine, and the like. The processor 218 may perform data processing, power control, input/output processing, sensor date processing, and/or any other functionality that enables the device 200 to treat mild cognitive impairment and dementia. The processor 218 may be coupled to the transceiver 220, which may be coupled to the transmit/receive element 222. While FIG. 2 depicts the processor 218 and the transceiver 220 as separate components, it will be appreciated that the processor 218 and the transceiver 220 may be integrated together in an electronic package or chip.

The transmit/receive element 222 may be configured to transmit data to, or receive data from a sever located in a medical institution. For example, medical instructions from a doctor/medical information sensed from a user may be received/transmitted from/to the server, via a base station over the air interface 216. In one embodiment, the transmit/receive element 222 may be an antenna configured to transmit and/or receive RF signals. In an embodiment, the transmit/receive element 222 may be an emitter/detector configured to transmit and/or receive IR, UV, or visible light signals, for example. In yet another embodiment, the transmit/receive element 222 may be configured to transmit and/or receive both RF and light signals. It will be appreciated that the transmit/receive element 222 may be configured to transmit and/or receive any combination of wireless signals. The transceiver 220 may be configured to modulate the signals that are to be transmitted by the transmit/receive element 222 and to demodulate the signals that are received by the transmit/receive element 222.

The processor 218 of the device 200 may be coupled to, and may receive user input data from, the speaker/microphone 224, the keypad 226, the display/touchpad 228 (e.g., a liquid crystal display (LCD) display unit or organic light-emitting diode (OLED) display unit) and/or the peripherals 238 (e.g., sensors or digital camera). The processor 218 may also output user data or digital instructions to the speaker/microphone 224, the keypad 226, the display/touchpad 228 and/or the peripherals 238. In addition, the processor 218 may access information from, and store data in, any type of suitable memory, such as the non-removable memory 230 and/or the removable memory 232. The non-removable memory 230 may include random-access memory (RAM), read-only memory (ROM), a hard disk, or any other type of memory storage device. The removable memory 232 may include a subscriber identity module (SIM) card, a memory stick, a secure digital (SD) memory card, and the like. In other embodiments, the processor 218 may access information from, and store data in, memory that is not physically located on the device 200, such as on a server or a home computer (not shown).

The processor 218 may receive power from the power source 234, and may be configured to distribute and/or control the power to the other components in the device 200. The power source 234 may be any suitable device for powering the device 200. For example, the power source 234 may include one or more dry cell batteries (e.g., nickel-cadmium (NiCd), nickel-zinc (NiZn), nickel metal hydride (NiMH), lithium-ion (Li-ion), etc.), solar cells, fuel cells, and the like.

The processor 218 may also be coupled to the GPS chipset 236, which may be configured to provide location information (e.g., longitude and latitude) regarding the current location of the device 200. In addition to, or in lieu of, the information from the GPS chipset 236, the device 200 may receive location information over the air interface 216 from a base station and/or determine its location based on the timing of the signals being received from two or more nearby base stations. It will be appreciated that the device 200 may acquire location information by way of any suitable location-determination method while remaining consistent with an embodiment.

The processor 218 may further be coupled to other peripherals 238, which may include one or more software and/or hardware modules that provide additional features, functionality and/or wired or wireless connectivity. For example, the peripherals 238 may include an accelerometer, an e-compass, a satellite transceiver, a digital camera (for photographs and/or video), a universal serial bus (USB) port, a vibration device, a television transceiver, a hands free headset, a Bluetooth® module, a frequency modulated (FM) radio unit, a digital music player, a media player, a video game player module, an Internet browser, a Virtual Reality and/or Augmented Reality (VR/AR) device, an activity tracker, and the like. The peripherals 238 may include one or more sensors. The sensors may be one or more of a gyroscope, an accelerometer, a hall effect sensor, a magnetometer, an orientation sensor, a proximity sensor, a temperature sensor, a time sensor; a geolocation sensor, an altimeter, a light sensor, a touch sensor, a magnetometer, a barometer, a gesture sensor, a biometric sensor, a humidity sensor and the like.

The processor 218 may perform a digital instruction generation, a sensing data collection, an execution input, an outcome analysis, communication with a database and a security function.

Based on the mechanism of action in and the therapeutic hypothesis and digital therapeutic hypothesis for amnestic MCI and AD, a doctor (e.g., a second user) may prescribe DTx, which are realized in a digital apparatus and/or an application for treating MCI and AD for the corresponding patient. In one example, the processor 218 may be configured to provide a prescription of the DTx to a patient as a specific behavioral instruction that the patient may execute based on the interaction between the neuro-humoral factors for MCI and AD and the patient's behaviors/environments. For example, the neuro-humoral factors may include, but are not limited to, sex steroid hormone, IGF-2, wnt/β-catenin, BAG1, CREB, inflammation factors, corticosteroids, neurohormones, and all types of neuro-humoral factors that may cause myopia can be considered.

The processor 218 may generate digital instructions based on the inputs from the doctor. In this case, the processor 218 may generate digital instructions based on information collected from/by the doctor when diagnosing a patient. Also, the processor 218 may generate digital instructions based on the information received from the patient. For example, the information received from the patient may include the patient's basal factors, medical information, and DTx literacy. In this case, the basal factors may include the patient's activity, heart rates, sleep, meals (e.g., nutrition and calories), and the like. The medical information may include the patient's electronic medical record (EMR), family history, genetic vulnerability, genetic susceptibility, and the like. The DTx literacy may include the patient's accessibility to the digital therapy instructions and the apparatus, an acceptance posture, and the like.

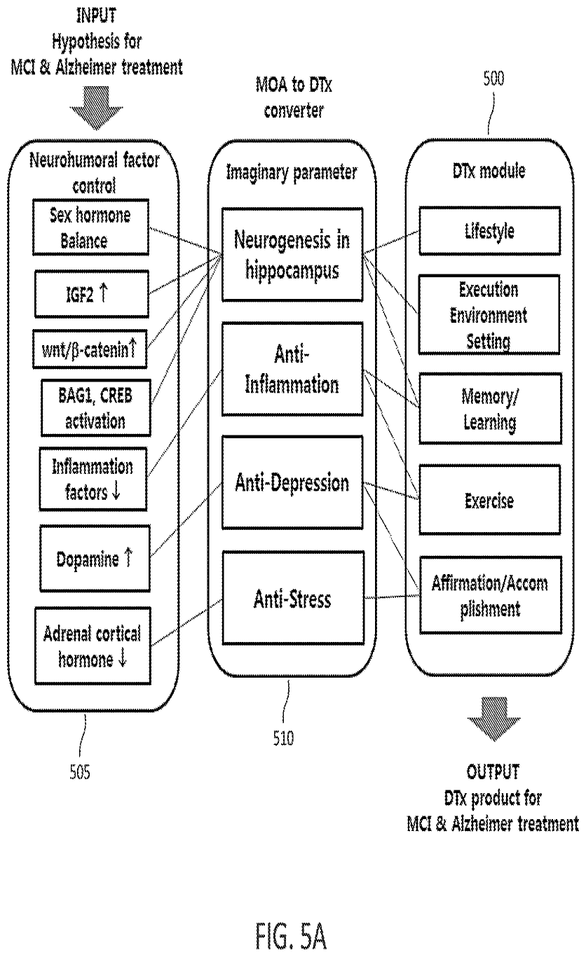

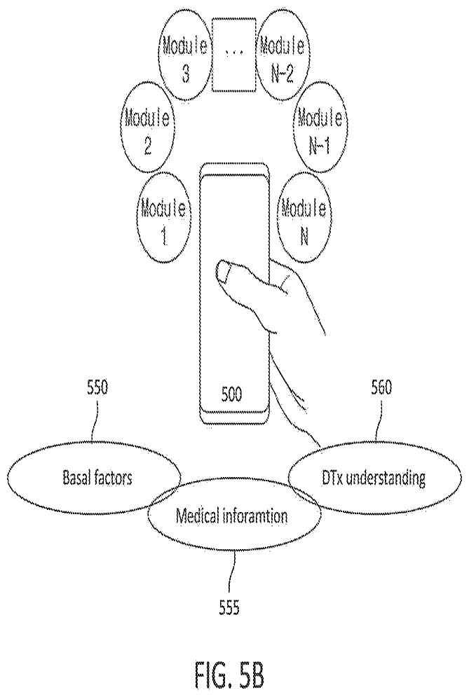

The processor 218 may reflect the MOA and the therapeutic hypothesis for MCI and AD in order to utilize one or more imaginary parameters and generate a digital module. In this case, the imaginary parameters may be deduced in term of neurogenesis in hippocampus, anti-inflammation, anti-stress and anti-depression, considering the patient's environments, behaviors, emotions, and cognition. The imaginary parameters will further be described in detail as shown in FIG. 5A.

The processor 218 may generate digital instructions particularly designed to allow a patient to have therapeutic effect, and provide the instructions to the patient. For example, the processor 218 may generate specific digital instructions in each DTx module.

The processor 218 may perform sensing data collection and execution input that can collect the patient's execution outcomes of the digital instructions. Specifically, the processor 218 is configured to sense the patient's adherence to the digital instructions and allow a patient to directly input the execution outcomes of the digital instructions, and thus serve to output the patient's execution outcomes of the digital instructions.

The processor 218 may collect the patient's behavior adherence or participation in predetermined periods, and report the patient's behavior adherence or participation to external systems. Therefore, a doctor may continue to monitor an execution course of the digital instructions through the application even when a patient does not directly visit a hospital.

The database can store the MOA and the therapeutic hypothesis of MCI and AD, the digital instructions provided to the user and the user's execution outcome data. Although it is not shown in FIG. 2, the database 050 may be included in the device 200 for treating MCI and AD. Alternatively or additionally, the database 050 may be provided in an external server.

Meanwhile, a series of loops including inputting the digital instructions, outputting the patient's execution outcomes of the digital instructions and evaluating the execution outcomes can be repeatedly executed several times. In this case, the processor may generate patient-customized digital instructions for this cycle by reflecting the patient's digital instructions provided in the previous cycle, the output values and the evaluation.

As described above, the device 200 for inhibiting progression of and treating amnestic MCI and AD can inhibit the progression of amnestic MCI and AD and provide improved therapeutic effect by deducing a MOA, therapeutic hypothesis and digital therapeutic hypothesis based on the neuro-humoral factors of amnestic MCI and AD. The device 200 may provide digital instructions based on these findings, and collect and analyze the execution outcomes.

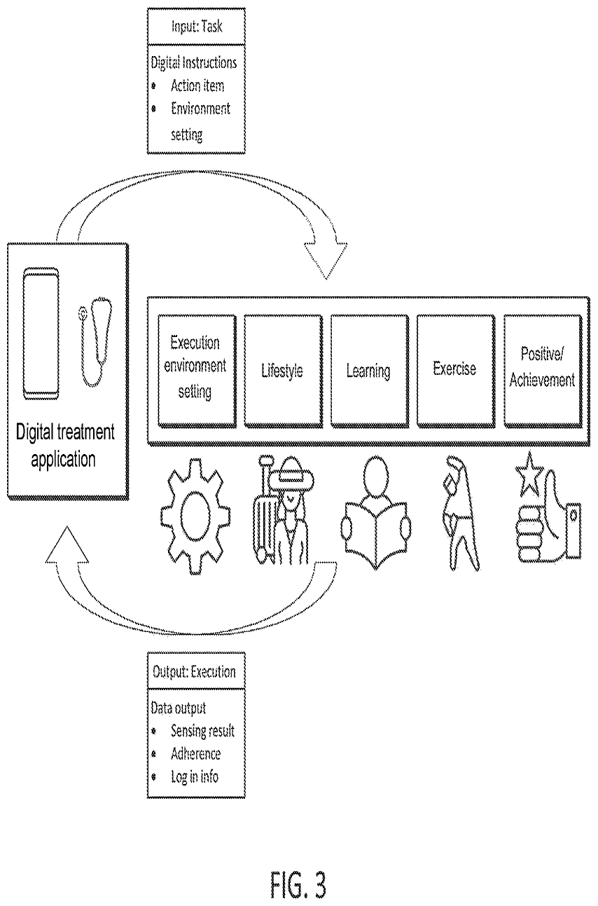

FIG. 3 is a diagram illustrating an example input and output loop of a user device for treating or inhibiting progression of MCI and AD, which may be used in combination with any of other embodiments described herein.

Referring to FIG. 3, the digital application for treating MCI and AD according to one embodiment may provide a corresponding digital prescription for a patient in the form of instructions. The execution outcomes of the corresponding digital instructions may be recursively entered into the application as an input.

The digital instructions provided to the patient may include specific action instructions and control of the patient's light environments. As shown in FIG. 3, the digital instructions may include, but are not limited to, execution environment setting, lifestyle, learning, exercise and positive/achievement.

The patient's execution outcomes of the digital instructions may comprise: (1) log-in/log-out information for instructions and execution; (2) adherence information sensed as passive data such as exercise, heart rates associated with the stress, a change in oxygen saturation, and the like; and (3) directly input information on the patient's execution outcomes.

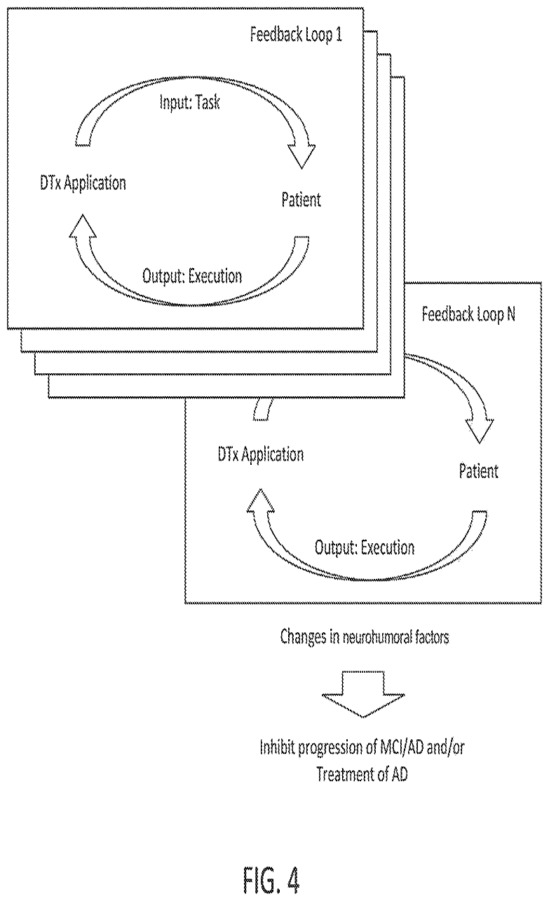

FIG. 4 is a diagram illustrating an example feedback loop for treating or inhibiting progression of and treating MCI and AD, which may be used in combination with any of other embodiments described herein.

Referring to FIG. 4, inhibition of the progression of and treatment of MCI and AD may be achieved by repeatedly executing the aforementioned single feedback loop of FIG. 3 multiple times to regulate the neuro-humoral factors.

In the case of MCI and AD, constant digital therapy and observation is needed. Due to these characteristics, inhibitory and therapeutic effects on progression of the MCI and AD may also be achieved by gradual improvement of an instruction-execution cycle in the feedback loop, compared to the simply repeated instruction-execution cycle without the feedback loop during the corresponding course of therapy. For example, the digital instructions and the execution outcomes for the first cycle are given as input values and output values in a single loop, but new digital instructions can be generated by reflecting input values and output values generated in this loop using a feedback process of the loop to adjust the input for the next loop when the feedback loop is executed N times. This feedback loop may be repeated to deduce patient-customized digital instructions and maximize a therapeutic effect at the same time.

As such, in the digital apparatus and the application, the patient's digital instructions provided in the previous cycle (e.g., (N−1)^{th} cycle), and the data on instruction execution outcomes may be used to calculate the patient's digital instructions and execution outcomes in the current cycle (e.g., N^{th} cycle). That is, the digital instructions in the next loop can be generated based on the patient's digital instructions and execution outcomes of the digital instructions calculated in the previous loop. In this case, various algorithms and statistical models may be used for the feedback process, when necessary.

As described above, in the digital apparatus and the application for treating MCI and AD, it is possible to optimize the patient-customized digital instructions suitable for the patient through the rapid feedback loop.

FIG. 5A is a diagram illustrating example modules to implement digital therapeutics for treating or inhibiting progression of MCI and AD, which may be used in combination with any of other embodiments described herein. FIG. 5B is a diagram illustrating example background factors that support a user device for treating or inhibiting progression of MCI and AD, which may be used in combination with any of other embodiments described herein.

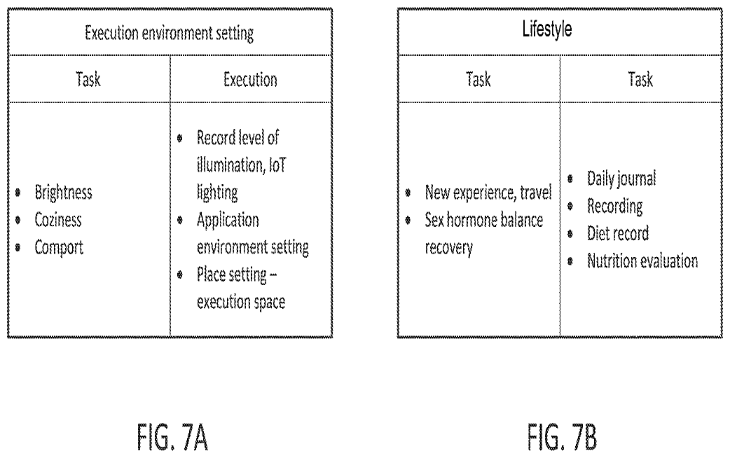

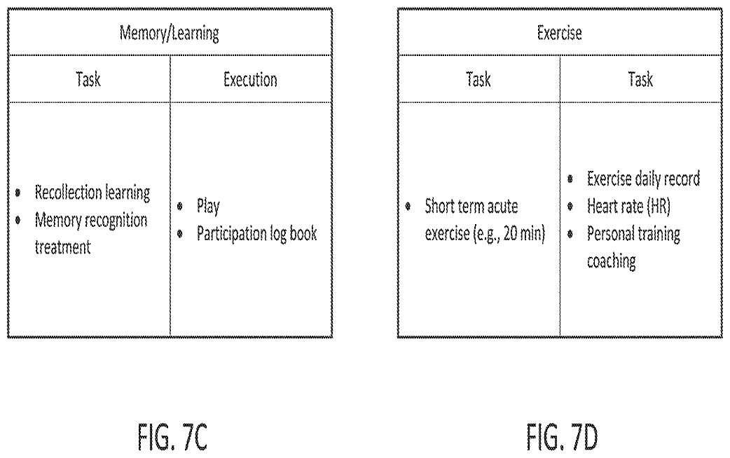

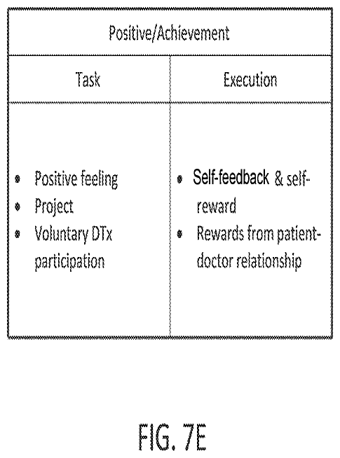

As shown in FIG. 5A, when the therapeutic hypothesis based on the MOA of MCI and AD is created, targeted neuro-humoral factors 505 (e.g., sex steroid hormone, IGF-2, wnt/β-catenin, BAG1, CREB, inflammation factors, corticosteroids and neurohormones, etc.) can be deduced. Imaginary parameters 510 may be utilized to allow specific instructions to correspond to the regulation of these neuro-humoral factors 505. Modules 500 to treat MCI and AD may be deduced using the “neuro-humoral factor-imaginary parameter-module” interrelation. Each of the modules 500 may be described in the form of modular instructions in further detail with reference to FIGS. 7A-E. In this case, each of the modules 500 is a basic design unit for digital therapeutics realized in the actual digital apparatus or the application, and is a collection of specific instructions.

Specifically, referring to FIG. 5A, the neuro-humoral factors 505 deduced based on the MOA and the therapeutic hypothesis for MCI and AD may include, but are not limited to, sex steroid hormone, IGF-2, wnt/β-catenin, BAG1, CREB, inflammation factors, corticosteroids and neurohormones. To treat MCI and AD, imbalance of the neurohumoral factors may be tackled by balancing sex steroid hormone, increasing secretion of IGF-2, balancing wnt/β-catenin, increasing secretion of BAG1 and CREB, decreasing secretion of inflammation factors and corticosteroids and increasing secretion of neurohormones.

To further elaborate on the “neuro-humoral factor-imaginary parameter-DTx module” interrelation from a molecular biological, neurophysiological and pathological perspective, each of the neuro-humoral factors may be illustrated in specified categories as i) neurogenesis in hippocampus related to declarative memory-semantic memory consolidation, ii) anti-inflammation related to episodic memory consolidation, iii) anti-stress and anti-depression.

Although almost all mammals, including human, undergo life-long continuous neurogenesis, generally active adult neurogenesis occurs only in limited brain areas where neurogenesis is especially apparent (the subgranular zone (SGZ) in the dentate gyrus of the hippocampus and the subventicular zone (SVZ) of the laternal venticles). Adult neurogenesis of the central nervous system (CNS) in the other areas is known to be highly limited under general physiological conditions.

What is first observed about the progression of amnestic MCI and AD is the damage in declarative memory-semantic memory. In order to treat or inhibit this progression, neurogenesis in hippocampus has been recently receiving attention, and genes and proteins related to neurogenesis may be used as the targets of dementia treatment drug developments.

Neurogenesis in hippocampus is related to sex steroid hormone, IGF-2, wnt/β-catenin, BAG1, CREB, or the like.

Decline in female hormone after menopause (i.e. imbalance in sex steroid hormone) illustrates relatively high dementia prevalence rate of women, which is as twice as higher than that of men. In the randomized controlled trial (RCT) on the impact of hormone replacement therapy (HRT) on cognitive function targeting postmenopausal women, only the HRT treatment group had demonstrated a significant increase in Montreal Cognitive Assessment (MoCA) score before and after the treatment, in other words, an increase in cognitive ability. MoCA is a type of screening test to detect MCI and AD and has been adopted in numerous hospitals.

Light is known to help enhance human cognitive function. However, the neurobiological mechanism of light in this positive sense has yet been acknowledged. Recently, research using a trained mouse on the timing of daily rhythms reported the relevance between long days and the increase of IGF-2 (locally secreted IGF), and between long days and the enhancement of long-term recognition memory in hippocampus.

Wnt signaling plays a significant role in nervous system development and adult synaptic plasticity. Particularly on learning and memory, Wnt signaling may take on a key role in normal functioning of hippocampus. Wnt/β-catenin E2/E4 balance relates to memory consolidation-storage-recollection through learning in the declarative memory area.

BAG gene relates to neurodegenerative diseases associated with ageing such as AD and is a crucial factor of neuronal differentiation. It may take a direct role in memory-related synaptic plasticity of CREB (cAMP Response Element Binding protein). BAG1/CREB relates to neurogenesis in hippocampus, and it has been recently reported that neurogenesis in hippocampus is controlled by the defense response resulted from treat- and extinction-signaling brain network.

By corresponding the aforementioned neuro-humoral factors 505 to the action instructions of the DTx module 500 using the imaginary parameter 510 of neurogenesis in hippocampus, dementia therapeutic effect can be derived from controlling/ameliorating the imbalance or deficiency of the neuro-humoral factor(s) through action instructions. The examples of specifically connecting the action instructions related to neurogenesis in hippocampus with the neuro-humoral factors can include amelioration of sex steroid hormone through dietary control, increase in IGF-2 secretion through good light environment, recovery of wnt/β-catenin balance through learning activities, and activation of BAG1/CREB brain neural network through experiences with treat- and extinction-situations.

Anti-inflammation is associated with inflammation factors. Inflammation factors may disturb consolidation of episodic memory and aggravate dementia by interrupting recollection of the declarative memory area. By corresponding inflammation factors to the action instructions of the DTx module using the imaginary parameter of anti-inflammation, dementia therapeutic effect can be derived from controlling/ameliorating the high inflammation level.

Anti-stress relates to corticosteroid hormone including cortisol and glucocorticoid. Anti-depression relates to neural hormone including dopamine, noradrenaline and somatostatin. The imbalance in corticosteroid steroid hormone and neural hormone negatively affect neurogenesis and leaning in hippocampus. By corresponding corticosteroid steroid hormone and neural hormone to the action instructions of the DTx module using the imaginary parameter of anti-stress and anti-depression, dementia therapeutic effect can be derived from controlling/ameliorating the imbalance in corticosteroid steroid hormone and neural hormone. The action instructions related to anti-inflammation, anti-stress and anti-depression can include safe and familiar execution environment, nutritious diet along with exercising and normal sleep inducing.

To summarize, the control of each neuro-humoral factor 505 may correspond to the DTx modules 500 by using imaginary parameters 510 such as neurogenesis in hippocampus, anti-inflammation, anti-stress and anti-depression. Also, the specific digital instructions for each module may be made based on the DTx modules. At the same time, the digital instructions can include execution environment setting and modules, such as lifestyle, learning, exercise, affirmation-achievement or similar module. However, the modules of this embodiment is not limited thereto.

The control of each of the neurohumoral factors 505 corresponded to the DTx module 500 using imaginary parameters 510 such as neurogenesis in hippocampus, anti-inflammation, anti-stress and anti-depression. And then, specific digital instructions may be generated for each module based on the converted modules. In this case, the digital instructions may include execution environment setups and modules (e.g., lifestyle, learning, exercise, affirmation-achievement), which can be output by monitoring. However, these modules 500 are given by way of illustration only, and are not intended to be limiting to the modules 500.

Referring to FIG. 5B, the background factors may be considered together in the design of the modules in the digital apparatus and the application for treating MCI and AD according to one embodiment of the present invention.

In this case, the background factors are elements necessary for correction of clinical trial outcomes during verification of the clinical effectiveness of digital therapy for MCI and AD. Specifically, in the background factors shown in FIG. 5B, the basal factors 550 may include, but are not limited to, activity, heart rates, sleep, meals (nutrition and calories), and the like. The medical information 555 may include, but are not limited to, EMR, family history, genetic vulnerability, and susceptibility, and the like. The medical information may have been written when a patient visited a hospital. The DTx literacy or understanding 560 may include, but is not limited to, the patient's accessibility to the digital therapy instructions and the apparatus, and an acceptance posture.

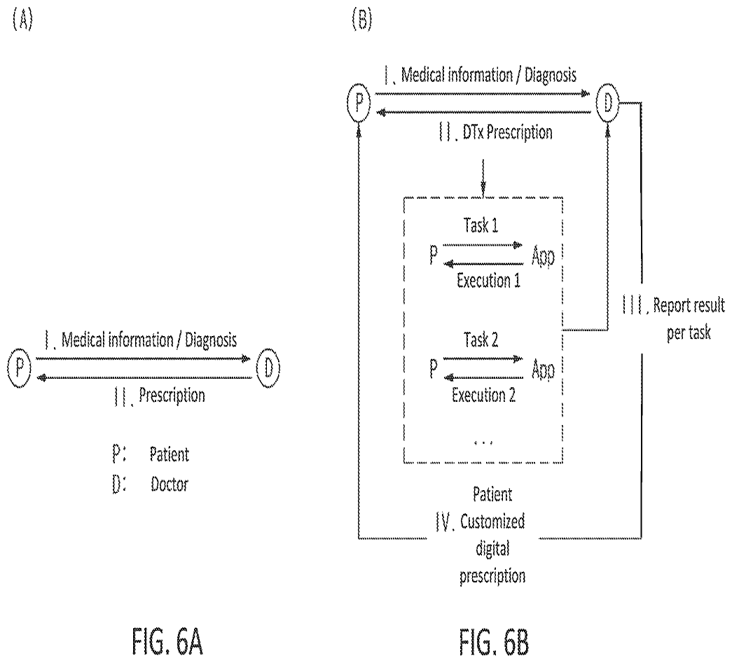

FIG. 6A is a diagram illustrating an example method of assigning a patient-customized prescription, which may be used in combination with any of other embodiments described herein. FIG. 6B is a diagram illustrating an example method of assigning a patient-customized digital prescription based on the analysis of a plurality of digital instructions and execution outcomes of the digital instructions, which may be used in combination with any of other embodiments described herein.

In this way, when the digital apparatus and the application for treating MCI and AD are used, a medical professional (e.g., doctor) may check the patient's instructions and execution outcomes for a given period and adjust the types of modules for treating MCI and AD, and the instructions for each module in a patient-customized manner, as shown in FIG. 6B.

FIG. 7A is a diagram illustrating an example digital instruction for an execution environment setting, which may be used in combination with any of other embodiments described herein. FIG. 7B is a diagram illustrating an example digital instruction for a lifestyle module, which may be used in combination with any of other embodiments described herein. FIG. 7C is a diagram illustrating an example digital instruction for a memory/learning module, which may be used in combination with any of other embodiments described herein. FIG. 7D is a diagram illustrating an example digital instruction for an exercise module, which may be used in combination with any of other embodiments described herein. FIG. 7E is a diagram illustrating an example digital instruction for a positive/achievement module, which may be used in combination with any of other embodiments described herein.

For digital therapy of MCI and AD, it is important for the participants to feel interest in the digital therapy and voluntarily take part in the therapy as continuous treatment. In this context, the modules may be configured by adding game elements to each module. In the digital apparatus and the application for inhibiting progression of and treating amnestic MCI and AD, as will be described below, each module comprises a collection of specific instructions.

Referring to FIG. 7A, specific examples of instructions for execution environment setups and a method of collecting output data are shown. In this case, the execution environment setups can be included as part of the configuration of the digital instruction generation as described above. The other modules shown in FIG. 7B to 7E may be executed under a bright, familiar and relaxing environment at execution environment setup of FIG. 7A.

Brightness setup may include, but is not limited to, setting the brightness of the digital instruction execution environment using an illuminance sensor or an IoT lamp, creating a living environment where a person can be exposed to bright surroundings, for example, for at least 16 hours a day.

Coziness or familiarity setup can include application environment setting (e.g., contents that might help the patient recall past memories such as the patient's favorite songs, scenes and lines from the patient's favorite movies, family pictures, documentary photography, etc.) and location setting configured to help patients recall memories in familiar and friendly surroundings for them during the execution of the instructions.

Comport or relaxation setup can include application environment setting (e.g., contents that might help relieve stress such as background wallpaper, music, tone, etc.) which provides a relaxing atmosphere to execute the instructions, and location setting which provides restfulness during the execution of the instructions.

FIGS. 7B-E show examples of specific instructions for each module, and methods of collecting output data. In these embodiments, modules can include lifestyle, learning, exercise and affirmation (or positive)-achievement modules.

Referring to FIG. 7B, specific examples of instruction for a lifestyle module, and a method of collecting output data are shown. In this case, the lifestyle module can be included as part of the configuration of the digital instruction generation as described above.

The new experience and travel instructions can include a trip to a new place to experience new environments once in every week or two week. The new experience and travel (instructions) can include recording (execution) using journals or various digital media.

Balance of sex hormones instruction aims at recovering from radical imbalance in sex steroid hormones due to ageing or climacterium. Particularly, climacteric women who have undergone radical changes in sex hormones are recommended to avoid HRT (hormone replacement therapy) or severe diet entailing radical loss of fat. Regaining the balance of sex hormones instruction through meals reflecting nutritional balance may include, but is not limited to, meal recording and nutrition evaluation (execution).

Referring to FIG. 7C, specific examples of instructions for a learning module and a method of collecting output data are shown. In this case, the learning module may be included as part of the configuration of the digital instruction generation as described above.

Specifically, the instructions of the learning module can include behavioral instructions that make patients constantly repeat and recall familiar things in a relaxed atmosphere. In case of the learning module, the first user's execution can be executed in the form of game record of log book which promotes memory and learning. Also, the comfortable environment for associative learning can be provided with the background, tone and music settings of the DTx. For example, the instructions of the learning module can induce the user to listen to relieving music or to smell good scents.

Referring to FIG. 7D, specific examples of instructions for an exercise module and a method of collecting output data are shown. In this case, the physical exercise module may be included as part of the configuration of the digital instruction generation as described above.

The exercise module may include a series of behavioral instructions which entail, for example, 20 minutes of acute exercise on a regular basis (e.g., three times a week).

Specifically, the behavioral instructions of the exercise module may include the method to collect the execution outcomes with the sensing data collection using neuronal biofeedback devices (e.g., EEG, ECG, EMG, EDG, etc.) or general sensors (e.g., activity, HR, etc.), or the method for the patient to directly input the execution outcomes using the execution input as described above. In this case, the exercise instructions can be organized according to the age and the physical condition of the patient, using the exercise therapy widely used by doctors or motor therapists.

In case of the exercise module, the first user can execute the behavioral instructions by writing an exercise journal, checking heart rates, or getting a personal training (PT) coach.

Referring to FIG. 7E, specific examples of instructions for an affirmation (or positive)/achievement module and a method of collecting output data are shown. In this case, the physical exercise module may be included as part of the configuration of the digital instruction generation as described above.

Specifically, the affirmation (or positive)/achievement module can include instructions that stimulate the secretion of dopamine through the patient's execution of tasks and the fulfillment of the completion. In this case, task completion instruction may be an instruction that makes patient feel a sense of accomplishment by completing the given tasks, wherein patients can update the tasks in accordance with the deadline and their voluntary participation can be induced. For example, the specific format of game can vary from learning, spot the difference or find the difference game, quiz, etc.

Particularly, that part realized as a format of quiz from the affirmation (or positive)/achievement module is further expected to enhance patient's health information literacy and DTx literacy. The enhancement in such abilities may be needed for a patient to constantly take part in the therapy and improve the patient's performance status.

In case of the affirmation (or positive)/achievement module, the first user can execute the task in ways such as self-feedback, self-reward, reward from a doctor based on the doctor-patient relationship and so on.

As mentioned above, long-term constant participation from the patients may be needed for the digital therapy. Diligent participation during the therapy can generate a compliment (reward) task in the affirmation (positive)/achievement module so that the patient can feel a sense of accomplishment. In the compliment task, patients who actively participate in the therapy can be compensated with reward and trust in patient-guardian and/or patient-doctor relationship.

Meanwhile, progression of MCI and AD and aging is closely related. Particularly, in the old aging period, there is a wide gap in standards set for new lifestyles, learning, exercise, and affirmation (or positive)/achievement, depending on age, sex, personality and preference. To bridge the gap, it is desirable to propose customized digital instructions for each module concerning the individual characteristics of each patient. Particularly, the instructions that require intercommunication with the application can be developed by combining with big data analysis and artificial intelligence analysis.

The digital instructions illustrated in FIG. 7B to FIG. 7E are given by way of illustration only, and are not intended to limit the present invention. For example, the digital instructions provided to the patient may be set in various manners, when necessary.

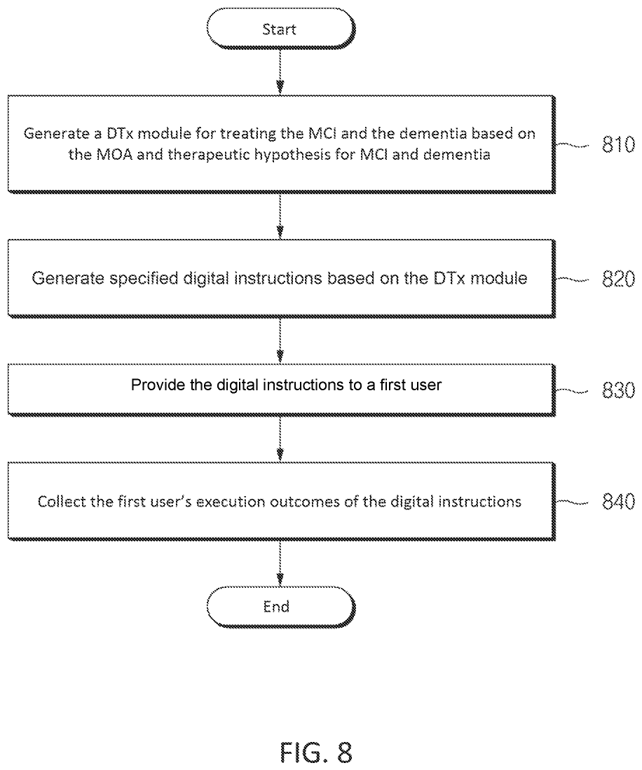

FIG. 8 is a diagram illustrating an example procedure using a digital application for treating or inhibiting progression of MCI and AD, which may be used in combination with any of other embodiments described herein.

Referring to FIG. 8, at step 810, the digital application for treating MCI and AD according to one embodiment can first generate a DTx module for treating MCI and AD based on the MOA and the therapeutic hypothesis of MCI and AD. In this case, the DTx module may be generated based on the neuro-humoral factors (e.g., sex steroid hormone, IGF-2, wnt/β-catenin, BAG1, CREB, inflammation factors, corticosteroids, neurohormones, etc.) for MCI and AD.

Alternatively or additionally, at step 810, the digital therapeutics module may be generated based on the inputs from a medical professional (e.g., doctor). In this case, a DTx module may be generated based on the information collected by the medical professional when diagnosing a patient, and the prescription outcomes recorded based on the information. Also, at step 810, the DTx module may be generated based on the information (e.g., basal factors, medical information, digital therapeutics literacy, etc.) received from the patient.

At step 820, specified digital instructions may be generated based on the DTx module. For example, a DTx module may be generated by applying imaginary parameters about the patient's environments and behavioral aspects (e.g., neurogenesis in hippocampus, anti-inflammation, anti-stress and anti-depression) to the MOA and the therapeutic hypothesis for MCI and AD. This DTx module is described with reference to FIGS. 5A and 5B, and thus description thereof will be omitted.

In this case, the digital instructions may be generated for at least one of execution environment setting, lifestyle, learning, exercise and affirmation (or positive)/achievement. Description of the execution environment setups and the specific digital instructions for each of the modules is as described in FIGS. 7A-E.

At step 830, the digital instructions may be provided to the patient. In this case, the digital instructions may be provided in the form of digital instructions which are associated with behaviors, emotions and cognition, and in which the patient's instruction adherence such as lifestyle and physical exercise may be monitored using a sensor, or provided in the form of digital instructions in which a patient is allowed to directly input the execution outcomes.

After the patient executes the presented digital instructions, at step 840, the patient's execution outcomes of the digital instructions may be collected. For example, the execution outcomes of the digital instructions may be collected by monitoring the patient's adherence to the digital instructions as described above, or allowing the patient to input the execution outcomes of the digital instructions.

Meanwhile, the digital application for treating MCI and AD according to one embodiment can repeatedly execute operations several times, wherein the operations include generating the digital instruction and collecting the patient's execution outcomes of the digital instructions. In this case, the generating of the digital instruction may include generating the patient's digital instructions for this cycle based on the patient's digital instructions provided in the previous cycle and the execution outcome data on the patient's collected digital instructions.

As described above, the MOA, the therapeutic hypothesis and the digital therapeutic hypothesis may be deduced considering neuro-humoral factors of the progression of amnestic MCI and AD. Patients will be given digital tasks based on these findings, and their execution and completion of tasks will be collected and analyzed in order to effectively inhibit the progression of amnestic MCI and AD and offer improved therapeutic effect.

Although the digital apparatus and the application for treating MCI and AD have been described in terms of MCI and AD therapy, the present invention is not limited thereto. For the other diseases other than MCI and AD, the digital therapy may be executed substantially in the same manner as described above.

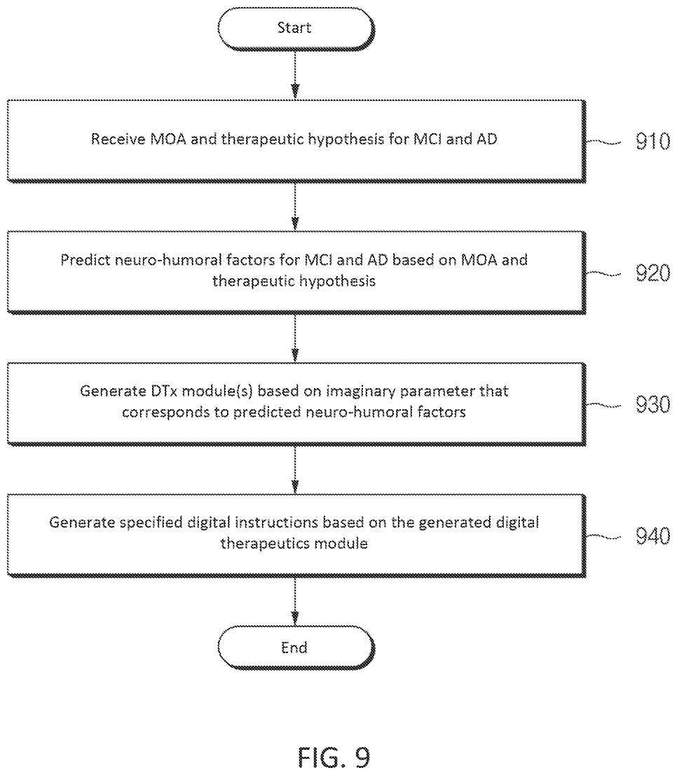

FIG. 9 is a diagram illustrating an example procedure for generating digital instructions to treat or inhibit progression of MCI and AD, which may be used in combination with any of other embodiments described herein.

FIG. 9 further describes the process of generating the module and the specified digital instructions for treating MCI and AD based on the MOA and the therapeutic hypothesis of MCI and AD as described in FIGS. 5A, 5B and 8.

At step 910, the MOA and the therapeutic hypothesis for MCI and AD can be input. In this case, the MOA and the therapeutic hypothesis for MCI and AD may be previously deduced through the literature search and expert reviews on the systematic related clinical trials on MCI and AD, as described above.

At step 920, neuro-humoral factors for MCI and AD may be predicted from the input MOA and therapeutic hypothesis. In this case, the neuro-humoral factors for MCI and AD predicted at step 920 may be deduced in the form of sex steroid hormone, IGF-2, wnt/β-catenin, BAG1, CREB, inflammation factors, corticosteroids, neurohormones, or the like. These neuro-humoral factors have been described in detail with reference to FIG. 5A, and thus description thereof will be omitted.

At step 930, a DTx module may be generated based on the predicted neuro-humoral factors corresponding to imaginary parameters. Here, the imaginary parameters may serve as converters that convert the neuro-humoral factors for MCI and AD into a DTx module, and this procedure is to set the physiological interrelation between the neuro-humoral factors and the environmental and behavioral factors, as shown in FIG. 5A.

At step 940, specified digital instructions may be generated based on the generated digital therapeutics module. In this case, the specific digital instructions may be generated by the execution environment setup, lifestyle, learning, exercise and affirmation (or positive)/achievement modules, which were described with reference to FIGS. 7A-E.

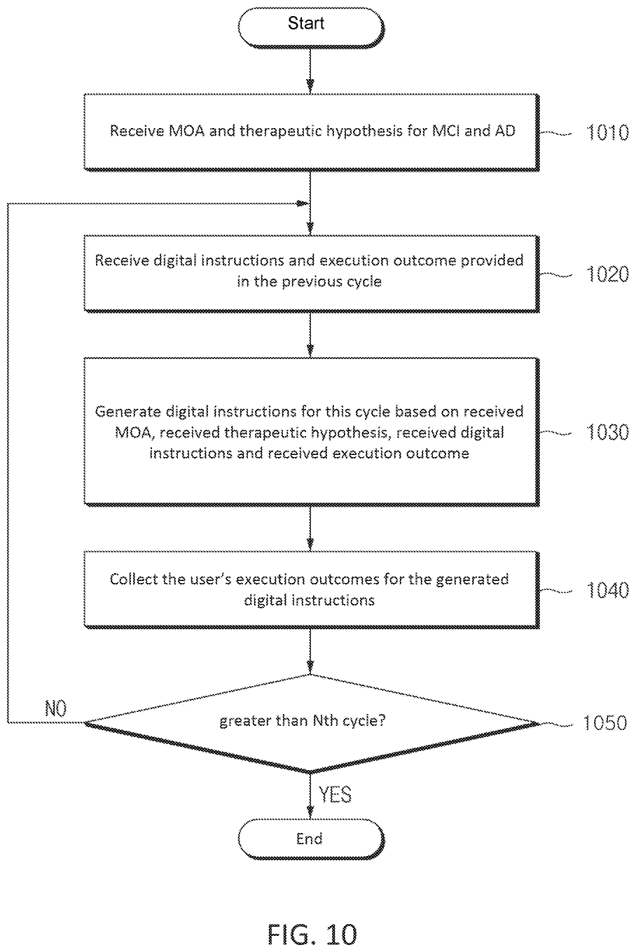

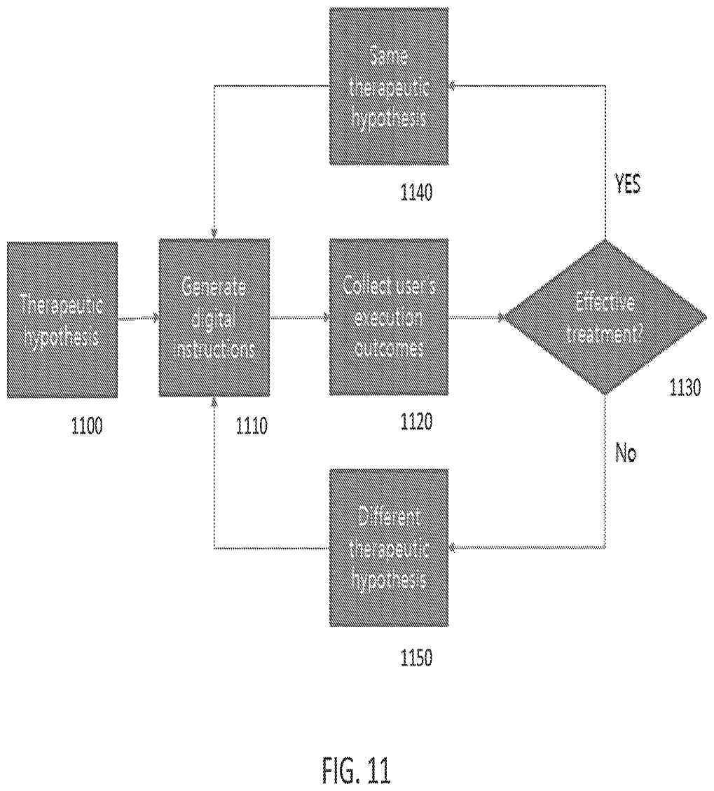

FIG. 10 is a diagram illustrating an example procedure that repeats the execution of digital instructions based on feedback for treating or inhibiting progression of MCI and AD, which may be used in combination with any of other embodiments described herein.

In FIG. 10, it is explained that generation of the digital instructions and collection of the execution outcomes at the digital application for treating MCI and AD are executed multiple times (e.g., N times). In this case, at step 1010, the MOA and the therapeutic hypothesis for MCI and AD may be input first. Also, at step 1020, the digital instructions provided in the previous cycle and the execution outcome data may be received. When the first cycle of execution is now in progress, step 1020 may be omitted because there are no previous data.

At step 1030, digital instructions for this cycle may be generated based on the input MOA and therapeutic hypothesis, the digital instruction provided in the previous cycle, and the execution outcome data. At step 1040, the user's execution outcomes of the generated digital instructions may be collected.

At step 1050, it is determined whether this cycle is greater than N^{th} cycle. If this cycle is less than the N^{th} cycle (i.e. NO), this may return again to step 1020, thus repeatedly executing step 1020 to step 1040. On the other hand, if this cycle is greater than the N^{th} cycle (i.e. YES), that is, when the generation of the digital instructions and the collection of the execution outcomes are executed N times, a feedback operation may be terminated. The number N may be preconfigured, predetermined or determined by a medical professional depending on the status of the patient.

FIG. 11 is a diagram illustrating another example procedure that repeats the execution of digital instructions based on feedback for treating or inhibiting progression of MCI and AD, which may be used in combination with any of other embodiments described herein.

Referring to FIG. 11, it is explained that generation of the digital instructions and collection of the execution outcomes at the application for treating MCI and AD are executed repeatedly. For example, at step 1110, the digital instructions may be generated if MOA and therapeutic hypothesis of MCI and AD are entered at step 1100. If the first cycle of execution is in progress, the first therapeutic hypothesis is input as 0 (e.g., TH 0) at step 1100. Also, at step 1120, the execution outcome data of the digital action instructions of the current cycle can be collected.

At step 1130, whether the current cycle has had sufficient therapeutic effect may be determined. If considered sufficient, the same therapeutic hypothesis is input for the next cycle at step 1140. If not, new therapeutic hypothesis may be generated based on the action instructions and execution outcomes of the current cycle at step 1150.

The algorithm above illustrates the feed process per cycle to attain optimal action instructions and execution outcomes from patients based on the MCI and AD therapeutic hypothesis which helps conclude optimal therapeutic effect for each patient.

The determination at step 1130 may be made by the doctor who monitors the digital instructions and execution outcomes. However, not all instruction-execution cycles need for the determination. Doctors can make judgments by collecting and analyzing the execution outcomes for different action instructions regularly such as on a pre-doctor-designated period basis, daily basis, weekly basis and monthly basis.

Step 1150 to step 1110 shows the process of creating a new therapeutic hypothesis to generate optimal action instructions for patient treatment. As described in FIGS. 5A and 5B, using the “neuro-humoral factor-imaginary parameter-module” interrelation, at step 1150 a new weight of neuro-humoral factors may be generated based on the new therapeutic hypothesis. The changes in the weight may be converted to the weight for each module of the DTx modules at step 1110. For example, through the feedback loop of step 1150-step 1110, combination for each module, repeat per module, execute time, intensity and others can be optimized.

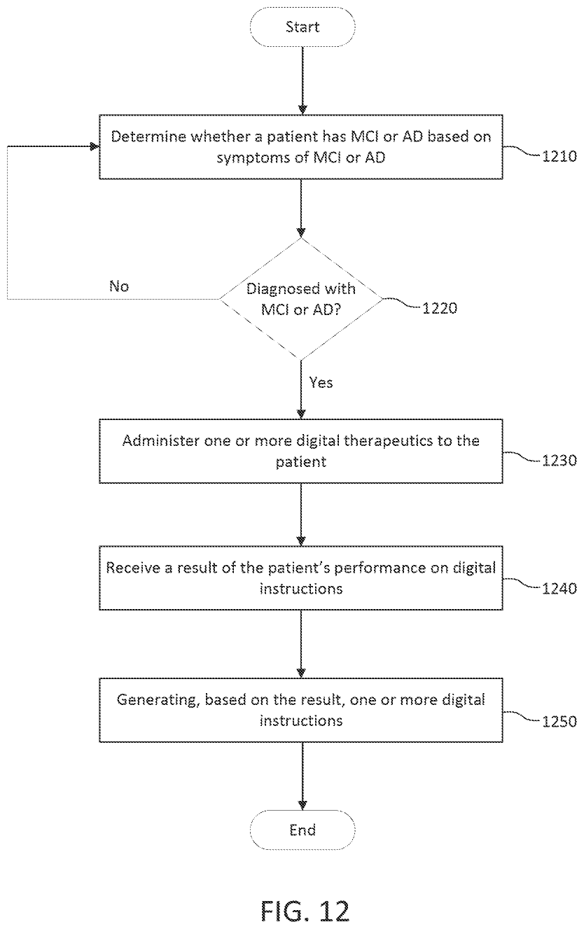

FIG. 12 is a diagram illustrating an example procedure for treating a patient with MCI or dementia by digital therapeutics, which may be used in combination with any of other embodiments described herein. As illustrated in FIG. 12, at step 1210, a medical professional (e.g., doctor) may determine determining whether the patient has MCI or dementia based on one or more symptoms of the MCI or the dementia. If the patient is diagnosed with MCI or dementia at step 1220, the medical professional may prescribe or administer one or more digital therapeutics to the patient to improve the patient's neurohumoral factors that caused the MCI or the dementia of the patient. The neurohumoral factors may include, but are not limited to, sex steroid hormone, insulin-like growth factor-2 (IGF-2), β-catenin in Wnt signaling, Bcl-2-associated athanogene 1 (BAG1), cAMP response element-binding protein (CREB), inflammation factors, corticosteroids, or neurohormones. More specifically, the corticosteroids may include cortisol and glucocorticoid, and the neurohormones may include dopamine, noradrenaline, and somatostatin.

The digital therapeutics prescribed or administered by the medical professional may comprise one or more digital instructions that are generated to treat imbalance of the neurohumoral factors based on the neurohumoral change among the neurohumoral factors. The neurohumoral imbalance (i.e. the imbalance of the neurohumoral factors) that may have caused the MCI or dementia to the patient may include, but are not limited to, a sex steroid hormone imbalance, a IGF-2 decrease, a β-catenin degradation, a BAG1 inactivation, a CREB inactivation, an increase in inflammation factors, a corticosteroids increase, or a neurohormone decrease.

The patient may perform the one or more digital instructions, thereby improving their neurohumoral imbalance. Examples of the digital instructions may include, but are not limited to, an execution environment setting, a lifestyle change, learning, exercising, being affirmative or an achievement task. The neurohumoral change caused by the patient's execution of the digital instructions may include, but are not limited to, neurogenesis in the patient's hippocampus, anti-inflammation, anti-stress, or anti-depression. Particularly, among the digital instructions, at least one of the execution environment setting, the lifestyle change, or the learning may be associated with the neurogenesis in the patient's hippocampus to treat at least one of the sex steroid hormone imbalance, the IGF-2 decrease, the β-catenin degradation, the BAG1 inactivation, or the CREB inactivation. Among the digital instructions, at least one of the execution environment setting or the learning may be associated with the anti-stress to treat the corticosteroids increase. Among the digital instructions, exercising may be associated with the anti-inflammation to treat the increase in inflammation factors. Among the digital instructions, the at least one of the execution environment setting, the being affirmative or the achievement task may be associated with the anti-depression to treat the neurohormone decrease.

At step 1240, the medical professional may receive or monitor the result of the patient's performance of the one or more digital instructions. The patient's performance of the one or more digital instructions may be repeated predetermined multiple times to improve the neurohumoral imbalance. After receiving or monitoring the patient's performance, at step 1250, different or the same digital instructions may be generated by the device or under the supervision of the medical professional. These digital instructions generated based on the result of the patient's performance may be used to treat imbalance of the plurality of neurohumoral factors.

In one embodiment, a patient may directly use a user device or apparatus to treat the patient's MCI or dementia. For example, the device may generate one or more digital therapeutics to improve the patient's neurohumoral factors that has caused the MCI or dementia. The digital therapeutics may comprise one or more digital instructions that are generated to treat at least one imbalance of the neurohumoral factors based on at least one neurohumoral change among the neurohumoral factors by the patient's performance of the one or more digital instructions. The device may also be configured to generate, based on the patient's performance of the one or more digital instructions, different or the same digital instructions to treat at least one imbalance of the neurohumoral factors of the patient.

The device may communicate with other devices (e.g., server) to report the result of the patient's performance on the one or more digital instructions. For example, the device used by the patient may transmit the result of patient's performance on the digital instructions to the server for the medical institution (e.g., hospital) or the device used by the medical professional. The device used by the patient may also receive, from other devices (e.g., server) second digital instructions. These second digital instructions may be the same or different digital instructions that the patient performed and reported. The second digital instructions may be instructed by the medical professional or the device used by the medical institution.

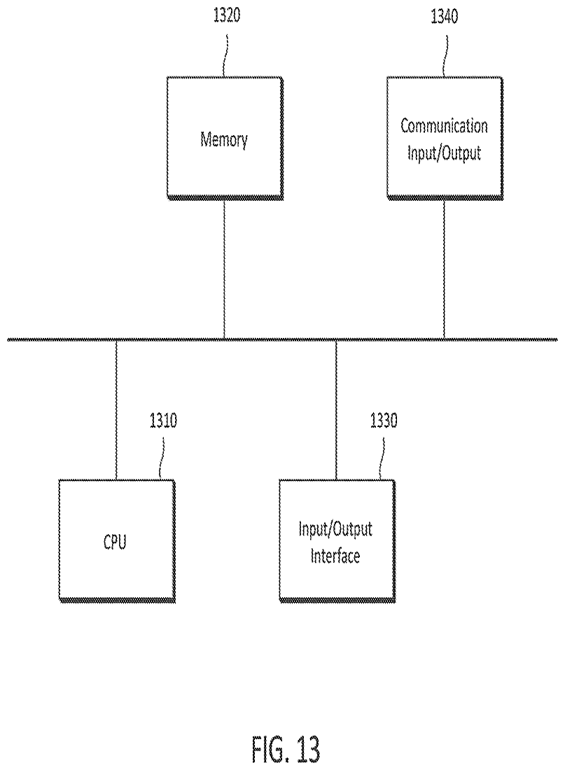

FIG. 13 is a diagram illustrating an example hardware configuration of a user device for treating or inhibiting progression of MCI and AD, which may be used in combination with any of other embodiments described herein.

Referring to FIG. 13, hardware of the digital apparatus for treating MCI and AD may include a CPU 1310, a memory 1320, an input/output I/F 1330, and a communication I/F 1340.

The CPU 1310 may be a processor configured to execute a digital program for treating MCI and AD stored in the memory 1320, process various data for treating digital MCI and AD, and execute functions associated with the digital therapy for MCI and AD. That is, the CPU 1310 may act to execute functions for each of the configurations by executing the digital program for treating MCI and AD stored in the memory 1320.

The memory 1320 can have a digital program for treating MCI and AD stored therein. Also, the memory 1320 may include the data used for the digital therapy for MCI and AD included in database, for example, the patient's digital instructions, instruction execution outcomes, the patient's medical information, and the like.

The memory 1320 may be a volatile memory or a non-volatile memory. If the memory 1320 is a volatile memory, RAM, DRAM, SRAM, and the like may be used as the memory 1320. If the memory 1320 is a nonvolatile memory, ROM, PROM, EAROM, EPROM, EEPROM, a flash memory, and the like may be used as the memory 620. Examples of the memories 1320 as listed above are given by way of illustration only, and are not intended to limit the present invention.

The input/output I/F 1330 can provide an interface in which input apparatuses (not shown) such as a keyboard, a mouse, a touch panel, and the like, and output apparatuses such as a display (not shown), and the like may be connected to the CPU 1310 to transmit and receive data.

The communication I/F 1340 is configured to transmit and receive various types of data to/from a server or other user device, and may be one of various apparatuses capable of supporting wire or wireless communication. For example, the types of data on the aforementioned digital behavior-based therapy may be received from a separately available external server through the communication I/F 1340.

As described above, the digital instructions generated to treat MCI and/or AD may be recorded in the memory 1320 and processed at the CPU 1310, for example, so that the digital instructions can be realized as a module configured to execute each of functional blocks.

Although all the components of the embodiment of the present invention are described to be combined and operated together, the invention is not necessarily limited to the embodiment of present invention. That is, within the scope of the purpose of the invention, the components can be selectively combined and operated.

The terms “comprises,” “comprising,” “includes” and/or “including,” used above, specify the presence of stated features, integers, steps, operations, elements, components and/or groups thereof, but do not preclude the presence or addition of one or more other features, integers, steps, operations, elements, components and/or groups thereof. All terminologies used herein including technical and scientific terminologies may hold the same meaning as are generally understood by those skilled in the art of technology of the present invention. Predefined, commonly used terminologies can hold the same or similar meaning to the contextual meaning of the relevant technology, and is not interpreted in an ideal or overly formal sense unless the context clearly indicates otherwise.

While the invention has been shown and described with reference to certain exemplary embodiments thereof, it will be understood by those skilled in the art that various changes in form and details may be made therein without departing from the spirit and scope of the invention as defined by the appended claims. In other words, the embodiments disclosed in the present invention are not intended to limit but rather to illustrate, and the exemplary embodiments do not limit the scope of the technical ideas of the invention. The scope of protection of the invention should be construed in accordance with the following claims, and all technical ideas within the same scope should be construed as being within the scope of the rights of this invention.

Although features and elements are described above in particular combinations, one of ordinary skill in the art will appreciate that each feature or element can be used alone or in any combination with the other features and elements. In addition, the methods described herein may be implemented in a computer program, software, or firmware incorporated in a computer-readable medium for execution by a computer or processor. Examples of computer-readable media include electronic signals (transmitted over wired or wireless connections) and computer-readable storage media. Examples of computer-readable storage media include, but are not limited to, a read only memory (ROM), a random access memory (RAM), a register, cache memory, semiconductor memory devices, magnetic media such as internal hard disks and removable disks, magneto-optical media, and optical media such as CD-ROM disks, and digital versatile disks (DVDs). A processor in association with software may be used to implement a radio frequency transceiver for use in a WTRU, UE, terminal, base station, RNC, or any host computer.

The present disclosure includes the following embodiments.

Embodiment 1. A method of treating a patient with mild cognitive impairment (MCI) or dementia by one or more digital therapeutics, the method comprising: administering the one or more digital therapeutics to the patient to improve a plurality of neurohumoral factors that cause the MCI or the dementia of the patient, wherein the one or more digital therapeutics comprise an instruction for an achievement task, detecting at least one neurohumoral change among the plurality of neurohumoral factors occurring because of the patient's performance of the one or more digital instructions, detecting the patient's adherence to the one or more digital instructions by at least one sensor selected from the group consisting of a gyroscope, an accelerometer, a hall effect sensor, a magnetometer, an orientation sensor, a proximity sensor, a temperature sensor, a time sensor, a geolocation sensor, an altimeter, a light sensor, a touch sensor, a magnetometer, a barometer, a gesture sensor, a biometric sensor, a humidity sensor, and generating one or more digital instructions to treat at least one imbalance of the plurality of neurohumoral factors based on the at least one neurohumoral change and the adherence.

Embodiment 2. The method of embodiment 1, wherein the plurality of neurohumoral factors includes at least one of sex steroid hormone, insulin-like growth factor-2 (IGF-2), β-catenin in Wnt signaling, Bcl-2-associated athanogene 1 (BAG1), cAMP response element-binding protein (CREB), inflammation factors, corticosteroids, or neurohormones.

Embodiment 3. The method of embodiment 2, wherein the corticosteroids include cortisol and glucocorticoid, and wherein the neurohormones include dopamine, noradrenaline, and somatostatin.

Embodiment 4. The method of embodiment 1, wherein the one or more digital instructions further includes at least one instruction selected from the group consisting of instructions for an execution environment setting, a lifestyle change, learning, exercising, and being affirmative.

Embodiment 5. The method of embodiment 4, wherein the at least one imbalance of the plurality of neurohumoral factors includes at least one of a sex steroid hormone imbalance, an IGF-2 decrease, a β-catenin degradation, a BAG1 inactivation, a CREB inactivation, an increase in inflammation factors, a corticosteroids increase, or a neurohormone decrease.

Embodiment 6. The method of embodiment 5, wherein the at least one neurohumoral change in the patient includes at least one of neurogenesis in the patient's hippocampus, anti-inflammation, anti-stress, or anti-depression.

Embodiment 7. The method of embodiment 6, wherein the at least one instruction further comprises instructions for the execution environment setting, the lifestyle change, or the learning, and the at least one instruction is associated with the neurogenesis in the patient's hippocampus to treat at least one of the sex steroid hormone imbalance, the IGF-2 decrease, the β-catenin degradation, the BAG1 inactivation, or the CREB inactivation.

Embodiment 8. The method of embodiment 6, wherein the at least one instruction further comprises instructions for the execution environment setting or the learning, and the at least one instruction is associated with the anti-stress to treat the corticosteroids increase.

Embodiment 9. The method of embodiment 6, wherein the at least one instruction further comprises instructions for the exercising, and the exercising is associated with the anti-inflammation to treat the increase in inflammation factors.

Embodiment 10. The method of embodiment 6, wherein the at least one instruction further comprises instructions for the execution environment setting, or the being affirmative, and the at least one instruction is associated with the anti-depression to treat the neurohormone decrease.

Embodiment 11. The method of embodiment 1, further comprising: receiving a result of the patient's performance of the one or more digital instructions that were repeated by the patient predetermined multiple times; and generating, based on the result, one or more digital instructions to treat at least one imbalance of the plurality of neurohumoral factors.

Embodiment 12. The method of embodiment 1, wherein the one or more digital therapeutics is performed by a wireless transmit/receive unit (WTRU).

Embodiment 13. A method of treating a subject having mild cognitive impairment (MCI) or dementia by one or more digital therapeutics, the method comprising: administering, by the subject, the one or more digital therapeutics to improve a plurality of neurohumoral factors that cause the MCI or the dementia of the subject, wherein the one or more digital therapeutics comprise first one or more digital instructions that are generated to treat at least one imbalance of the plurality of neurohumoral factors based on at least one neurohumoral change among the plurality of neurohumoral factors by performing the one or more digital instructions, and the first one or more digital instructions include an instruction for an achievement task.

Embodiment 14. The method of embodiment 13, wherein the plurality of neurohumoral factors includes at least one of sex steroid hormone, insulin-like growth factor-2 (IGF-2), β-catenin in Wnt signaling, Bcl-2-associated athanogene 1 (BAG1), cAMP response element-binding protein (CREB), inflammation factors, corticosteroids that include cortisol and glucocorticoid, or neurohormones that include dopamine, noradrenaline, and somatostatin.

Embodiment 15. The method of embodiment 13, wherein the first one or more digital instructions further includes at least one of an execution environment setting, a lifestyle change, learning, exercising, or being affirmative

[symbol]

Embodiment 16. The method of embodiment 13, wherein the at least one imbalance of the plurality of neurohumoral factors includes at least one of a sex steroid hormone imbalance, a IGF-2 decrease, a β-catenin degradation, a BAG1 inactivation, a CREB inactivation, an increase in inflammation factors, a corticosteroids increase, or a neurohormone decrease, and wherein the at least one neurohumoral change in the subject includes at least one of neurogenesis in the patient's hippocampus, anti-inflammation, anti-stress or anti-depression.

Embodiment 17. The method of embodiment 13, further comprising: receiving, by the subject, based on a result of performing the first one or more digital instructions, a second one or more digital instructions; and performing, based on the result, the second one or more digital instructions to treat at least one imbalance of the plurality of neurohumoral factors.

Embodiment 18. An apparatus for use by a subject having mild cognitive impairment (MCI) or dementia, the apparatus comprising: a processor configured to generate one or more digital therapeutics to improve a plurality of neurohumoral factors that cause the MCI or the dementia of the subject, wherein the one or more digital therapeutics comprise one or more digital instructions that are generated to treat at least one imbalance of the plurality of neurohumoral factors based on at least one neurohumoral change among the plurality of neurohumoral factors by the subject's performance of the one or more digital instructions.

Embodiment 19. The apparatus of embodiment 18, wherein the processor is further configured to generate, based on the subject's performance of the one or more digital instructions, second one or more digital instructions to treat at least one imbalance of the plurality of neurohumoral factors of the subject.

Embodiment 20. The apparatus of embodiment 18, further comprising: a transmitter configured to transmit, to another apparatus of a medical entity, a result of the subject's performance of the one or more digital instructions; and a receiver configured to receive, from the another apparatus, second one or more digital therapeutics that includes second one or more digital instructions generated based on the result in order to treat at least one imbalance of the plurality of neurohumoral factors.

Embodiment 21. The method of embodiment 1, wherein the one or more digital instructions further comprise an instruction for an execution environment setting, the execution environment setting includes brightness, coziness, or comfort.

Embodiment 22. The method of embodiment 1, wherein the one or more digital instructions further comprise an instruction for a lifestyle change, the lifestyle change includes new experience, traveling, or sex hormone balance recovery.

Embodiment 23. The method of embodiment 1, wherein the one or more digital instructions further includes instructions for an execution environment setting, a lifestyle change, learning, exercising, and being affirmative.

Cancer cachexia is characterized by an ongoing loss of skeletal muscle mass (with or without loss of fat mass) that cannot be fully reversed by conventional nutrition. Current therapies for cachexia include medication aimed at retarding or halting progression of the disorder. Treatments include, for example, orexigenic agents (i.e., appetite stimulants), corticosteroids, cannabinoids, serotonin antagonists, prokinetic agents, androgens and anabolic agents, anticytokine agents, non-steroidal anti-inflammatory drugs, and regulators of circadian rhythm, with most therapies directed to treating the underlying or associated condition (e.g., cancer). However, such treatment (e.g., using small molecules, biologics, etc.) is often compromised by the patient's inability to tolerate the treatment due to their cachexia. Thus, there is a need in the art for improved treatments (e.g., digital therapeutics) for wasting disorders, such as cachexia.

The present disclosure relates to digital therapeutics (hereinafter referred to as DTx) intended for cancer cachexia therapy. The present disclosure also relates to systems that integrate digital therapeutics with one or both of a healthcare provider portal and an administrative portal to treat cancer cachexia in a patient. Some embodiments of the present disclosure may comprise deducing a mechanism of action (hereinafter referred to as MOA) in cancer cachexia, and establishing a therapeutic hypothesis and a digital therapeutic hypothesis for inhibiting progression of cancer cachexia, and treating the cancer cachexia based on these findings.

In some aspects, the present disclosure provides a method of treating cancer cachexia in a subject in need thereof, the method comprising: providing, by an electronic device to the subject, one or more first modules selected from the group consisting of a voluntary skeletal muscle exercise module, a vagal nerve stimulation module, an aerobic exercise module, and a relaxation module, each of the one or more first modules comprising one or more first instructions for the subject to follow, wherein the electronic device (i) comprises a sensor sensing adherence by the subject to the first instructions of the one or more first modules, (ii) transmits adherence information, based on the adherence, to a server, and (iii) receives one or more second instructions from the server based on the adherence information; and providing, by the electronic device to the subject, one or more second modules selected from the group consisting of a voluntary skeletal muscle exercise module, a vagal nerve stimulation module, an aerobic exercise module, and a relaxation module, the one or more second modules comprising the one or more second instructions.

In some aspects, the present disclosure provides a computing system for treating cancer cachexia in a subject in need thereof, comprising a display configured to provide, to the subject, one or more first modules selected from the group consisting of a voluntary skeletal muscle exercise module, a vagal nerve stimulation module, an aerobic exercise module, and a relaxation module, each of the one or more first modules comprising one or more first instructions for the subject to follow; a sensor configured to sense adherence by the subject to the instructions of the one or more first modules; a transmitter configured to transmit adherence information, based on the adherence, to a server; and a receiver configured to receive, from the server, one or more second instructions based on the adherence information, wherein the display is further configured to provide, to the subject, one or more second modules selected from the group consisting of a voluntary skeletal muscle exercise module, a vagal nerve stimulation module, an aerobic exercise module, and a relaxation module, each of the one or more second modules comprising the one or more second instructions.

In some aspects, the present disclosure provides a non-transitory computer readable medium having stored thereon software instructions for treating cancer cachexia in a subject in need thereof that, when executed by a processor, cause the processor to: display, by an electronic device to the subject, one or more first modules selected from the group consisting of a voluntary skeletal muscle exercise module, a vagal nerve stimulation module, an aerobic exercise module, and a relaxation module, each of the one or more first modules comprising instructions for the subject to follow; sense, by a sensor in the electronic device, adherence by the subject to the instructions of the one or more first modules; transmit, by the electronic device, adherence information, based on the adherence, to a server; receive, from the server, one or more second instructions based on the adherence information; and display, to the subject, one or more second modules selected from the group consisting of a voluntary skeletal muscle exercise module, a vagal nerve stimulation module, an aerobic exercise module, and a relaxation module, the one or more second modules comprising the one or more second instructions.

In some aspects, the present disclosure provides a system for treating cancer cachexia in a subject, comprising: a digital apparatus configured to execute a digital application comprising one or more first modules, for treating cancer cachexia in a subject, wherein the digital apparatus comprises a sensor for sensing adherence by the subject to a first set of instructions of the one or more first modules; a healthcare provider portal configured to provide one or more options to a healthcare provider to perform one or more tasks to prescribe treatment for the cancer cachexia in the subject based on information received from the digital application; and an administrative portal configured to provide one or more options to an administrator of the system to perform one or more tasks to manage access to the system by the healthcare provider.

In some embodiments, the digital application for treating cancer cachexia instructs a processor of the digital apparatus to execute operations comprising:

generating digital therapeutic modules for treating cancer cachexia based on a mechanism of action in and a therapeutic hypothesis for the cancer cachexia. In some embodiments, the generating of the digital therapeutic modules comprises generating the digital therapeutic modules based on biochemical factors related to the cancer cachexia. In some embodiments, the one or more first modules comprise the voluntary skeletal muscle exercise module, and the voluntary skeletal muscle exercise module comprises one or more first instructions to increase MGF/IGF-1 secretion in the subject. In some embodiments, the one or more first instructions comprise instructions for aerobic, resistance, and/or concurrent exercise. In some embodiments, the one or more first modules comprise the vagal nerve stimulation module, and the vagal nerve stimulation module comprises one or more first instructions to reduce inflammation in the subject. In some embodiments, the one or more first modules comprise the vagal nerve stimulation module, and the vagal nerve stimulation module comprises at least one instruction selected from the group consisting of sense stimulation instructions for sight, sound, touch, taste, and smell. In some embodiments, the vagal nerve stimulation module comprises one or more sense stimulation instructions for sight, and the one or more sense stimulation instructions for sight include one or more instructions to view one or more figures (define in spec to include specific photos) to stimulate autonomic nervous system. In some embodiments, the electronic device receives and displays the figures. In some embodiments, the vagal nerve stimulation module comprises one or more sense stimulation instructions for sound, and the one or more sense stimulation instructions for sound include one or more instructions to hear one or more sounds to cause horror or relaxation. In some embodiments, the electronic device receives and plays the sounds. In some embodiments, the vagal nerve stimulation module comprises one or more sense stimulation instructions for touch, and the one or more sense stimulation instructions for touch include one or more instructions for abdominal breathing, controlling rate of breathing, cold massage, coughing, and skin massage. In some embodiments, the vagal nerve stimulation module comprises one or more sense stimulation instructions for touch, and the one or more sense stimulation instructions for touch include one or more instructions for abdominal breathing. In some embodiments, the vagal nerve stimulation module comprises one or more sense stimulation instructions for taste, and the one or more sense stimulation instructions for taste include one or more instructions to eat food to stimulate digestive glands in the subject. In some embodiments, the electronic device receives and displays information related to the food. In some embodiments, the vagal nerve stimulation module comprises one or more sense stimulation instructions for smell, and the one or more sense stimulation instructions for smell include one or more instructions to stimulate digestive glands and/or to relax. In some embodiments, the electronic device is configured to release a scent for aroma therapy. In some embodiments, the one or more first modules comprise the aerobic exercise module, and the aerobic exercise module comprises one or more first instructions to increase adiponectin secretion in the subject. In some embodiments, the one or more first instructions comprise one or more instructions for walking, biking, aerobic dance and/or swimming. In some embodiments, the one or more first modules comprise the relaxation module, and the relaxation module comprises one or more first instructions to increase adiponectin secretion in the subject. In some embodiments, the one or more first instructions comprise one or more meditation instructions. In some embodiments, said one or more first instructions comprise one or more sound instructions to hear relaxing sound. In some embodiments, the device receives and plays the relaxing sound. In some embodiments, the subject is an early cancer patient. In some embodiments, the subject has a cancer mass having a diameter of 3 cm or less. In some embodiments, the subject is a late cancer patient, and the system excludes providing a voluntary skeletal muscle exercise module. In some embodiments, the subject has a cancer mass having a diameter of more than 3 cm, and the system excludes providing a voluntary skeletal muscle exercise module. In some embodiments, the subject has moderate muscle atrophy, and the one or more first modules consists of the voluntary skeletal muscle exercise module, the vagal nerve stimulation module, the aerobic exercise module, and the relaxation module. In some embodiments, the subject has moderate muscle atrophy, and the one or more first modules consists of the vagal nerve stimulation module, the aerobic exercise module, and the relaxation module. In some embodiments, the subject has moderate muscle atrophy (in spec, define the moderate muscle atrophy as still being able to walk). In some embodiments, the subject has severe muscle atrophy (in spec, define the severe muscle atrophy as not being able to walk), and the system excludes providing a voluntary skeletal muscle exercise module and further excludes providing an aerobic exercise module. In some embodiments, the one or more first modules consists of the vagal nerve stimulation module and the relaxation module. In some embodiments, the digital application transmits data to a server, and wherein the server receives the one or more second instructions from an external reviewer. In some embodiments, the external reviewer comprises a health professional. In some embodiments, the external reviewer comprises an artificial intelligence (AI). In some embodiments, the sensor comprises one or more of: a camera, an accelerometer, a magnetometer, a light sensor, a microphone, a proximity sensor, a touch sensor, a gyroscope, a Global Positioning System (GPS) sensor, an ambient light sensor, a fingerprint sensor, a pedometer, a heart rate sensor, and a thermometer. In some embodiments, the sensor comprises a touch sensor, and the subject provides the adherence information to the electronic device using the touch sensor. In some embodiments, the one or more options provided to the healthcare provider are selected from the group consisting of adding or removing the subject, viewing or editing personal information for the subject, viewing adherence information for the subject, viewing a result of the subject for one or more at least partially completed digital therapeutic modules, prescribing one or more digital therapeutic modules to the subject, altering a prescription for one or more digital therapeutic modules, and communicating with the subject. In some embodiments, the one or more options comprise the viewing or editing personal information for the subject, and the personal information comprises one or more selected from the group consisting of an identification number for the subject, a name of the subject, a date of birth of the subject, an email of the subject, an email of the guardian of the subject, a contact phone number for the subject, a prescription for the subject, and one or more notes made by the healthcare provider about the subject. In some embodiments, the personal information comprises the prescription for the subject, and the prescription for the subject comprises one or more selected from the group consisting of a prescription identification number, a prescription type, a start date, a duration, a completion date, a number of scheduled or prescribed digital therapeutic modules to be performed by the subject, and a number of scheduled or prescribed digital therapeutic modules to be performed by the subject per day. In some embodiments, the one or more options comprise the viewing the adherence information, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules. In some embodiments, the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, an indicator of whether the scheduled or prescribed digital therapeutic module was fully or partially completed, and an exercise intensity (EI). In some embodiments, the one or more options provided to the administrator of the system are selected from the group consisting of adding or removing the healthcare provider, viewing or editing personal information for the healthcare provider, viewing or editing de-identified information of the subject, viewing adherence information for the subject, viewing a result of the subject for one or more at least partially completed digital therapeutic modules, and communicating with the healthcare provider. In some embodiments, the one or more options comprise the viewing or editing the personal information, and the personal information of the healthcare provider comprises one or more selected from the group consisting of an identification number for the healthcare provider, a name of the healthcare provider, an email of the healthcare provider, and a contact phone number for the healthcare provider. In some embodiments, the one or more options comprise the viewing or editing the de-identified information of the subject, and the de-identified information of the subject comprises one or more selected from the group consisting of an identification number for the subject, and the healthcare provider for the subject. In some embodiments, the one or more options comprise the viewing the adherence information for the subject, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules. In some embodiments, the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, an indicator of whether the scheduled or prescribed digital therapeutic module was fully or partially completed, and an exercise intensity (EI). In some embodiments, the digital application further comprises a push alarm for one or more of reminding the subject complete a digital therapeutic module. In some embodiments, the digital apparatus comprises a digital instruction generation unit configured to generate digital therapeutic modules for treating cancer cachexia, generate digital instructions based on the digital therapeutic modules, and provide the digital instructions to the subject; In some embodiments, an outcome collection unit configured to collect the subject's execution outcomes of the digital instructions. In some embodiments, the digital instruction generation unit generates the digital therapeutic modules based on biochemical factors related to the cancer cachexia onset. In some embodiments, the biochemical factors comprise insulin-like growth factor 1 (IGF1) and hypoxia-inducible factor 1 (HIF1). In some embodiments, the digital instruction generation unit generates the digital therapeutic modules based on the inputs from the healthcare provider. In some embodiments, the digital instruction generation unit generates the digital therapeutic modules based on information received from the subject. In some embodiments, the information is received from the subject comprises at least one of basal factors, medical information, and digital therapeutics literacy of the subject, the basal factors including the subject's activity, heart rate, sleep, and diet (including nutrition and calories), the medical information including the subject's electronic medical record (EMR), family history, genetic vulnerability, and genetic susceptibility, and the digital therapeutics literacy including the subject's accessibility, and technology adoption to the digital therapeutics and the apparatus. In some embodiments, the digital instruction generation unit generates the digital therapeutic modules matching to imaginary parameters which correspond to the mechanism of action in and the therapeutic hypothesis for the cancer cachexia. In some embodiments, the imaginary parameters are deduced in relation to the subject's environment, behaviors, emotions, and cognition. In some embodiments, the outcome collection unit collects the execution outcomes of the digital instructions by monitoring the subject's adherence to the digital instructions or allowing the subject to directly input the subject's adherence to the digital instructions. In some embodiments, the generation of the digital instructions at the digital instruction generation unit and the collection of the subject's execution outcomes of the digital instructions at the outcome collection unit are repeatedly executed several times with multiple feedback loops, and the digital instruction generation unit generates the subject's digital instructions for this cycle based on the subject's digital instructions in the previous cycle and the execution outcome data on the subject's digital instructions in the previous cycle collected at the outcome collection unit.

Hereinafter, exemplary embodiments of the present disclosure will be described in detail. However, the present disclosure is not limited to the embodiments disclosed below, but may be implemented in various forms. The following embodiments are described in order to enable those of ordinary skill in the art to embody and practice embodiments of the present disclosure.

Although the terms first, second, etc. may be used to describe various elements, these elements are not limited by these terms. These terms are only used to distinguish one element from another. For example, a first element could be termed a second element, and, similarly, a second element could be termed a first element, without departing from the scope of exemplary embodiments. The term “and/or” includes any and all combinations of one or more of the associated listed items.

The terminology used herein is for the purpose of describing particular embodiments only and is not intended to be limiting of exemplary embodiments. The singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, components and/or groups thereof, but do not preclude the presence or addition of one or more other features, integers, steps, operations, elements, components and/or groups thereof.

As used herein, the term “about” generally refers to a particular numeric value that is within an acceptable error range as determined by one of ordinary skill in the art, which will depend in part on how the numeric value is measured or determined, i.e., the limitations of the measurement system. For example, “about” may mean a range of ±20%, ±10%, or ±5% of a given numeric value.

As used herein, “muscle atrophy” can refer to a disease in which muscles of the body (e.g., legs and arms) are gradually atrophied, sometimes symmetrically. Muscle atrophy may accompany the occurrence of cancer, aging, renal diseases, genetic diseases, and various chronic diseases.

As used herein, the term “cancer cachexia” can refer to a condition characterized by an ongoing loss of skeletal muscle mass (with or without loss of fat mass) that cannot be fully reversed by conventional nutrition.

With reference to the appended drawings, exemplary embodiments of the present disclosure will be described in detail below. To aid in understanding the present disclosure, like numbers refer to like elements throughout the description of the figures, and the description of the same elements will be not reiterated.

Development of new drugs starts with confirming a medial demand in situ, proposing a mechanism of action based on the expert reviews and meta-analysis on the corresponding disease, and deducing a therapeutic hypothesis based on the expert reviews and the meta-analysis. Also, after a library of drugs whose therapeutic effects are expected is prepared based on the therapeutic hypothesis, a candidate material is found through screening, and the corresponding candidate material is subjected to optimization and preclinical trials to check its effectiveness and safety from a preclinical stage, thereby deciding the candidate material as a final candidate drug. To mass-produce the corresponding candidate drug, a CMC (chemistry, manufacturing, and control) process is also established, a clinical trial is carried out on the corresponding candidate drug to verify a mechanism of action and a therapeutic hypothesis of the candidate drug, thereby ensuring the clinical effectiveness and safety of the candidate drug.

From the point of view of this patent, drug targeting and signaling, which fall upstream of the development of new drugs, have many uncertainties. In many cases, because the drug targeting and signaling take a methodology of putting together the outcomes, which have been reported in the art, and interpreting the outcomes, it may be difficult to guarantee the novelty of disclosure. On the contrary, the disclosure of drugs capable of regulating the drug targeting and signaling to treat a disease requires the highest level of creativity except for the field of some antibody or nucleic acid (DNA, RNA) therapeutics in spite of the development of research methodology for research and development of numerous new drugs. As a result, the molecular structures of the drugs are the most critical factors that constitute the most potent substance patent in the field of new drugs.

Unlike the drugs whose rights are strongly protected through this substance patent, digital therapeutics are basically realized using software. Due to the nature of the digital therapeutics, the rational design of digital therapeutics against the corresponding disease, and the software realization of the digital therapeutics based on the rational design may be considered to be an improved process of disclosure to be protected as a patent when considering the clinical verification and approval processes as the therapeutics.

That is, the core of the digital therapeutics as in the present disclosure depends on the rational design of digital therapeutics suitable for treatment of the corresponding disease, and the development of specific software capable of clinically verifying the digital therapeutics based on the rational design. Hereinafter, a digital apparatus and an application for treating cancer cachexia according to the present disclosure realized in this aspect will be described in detail.

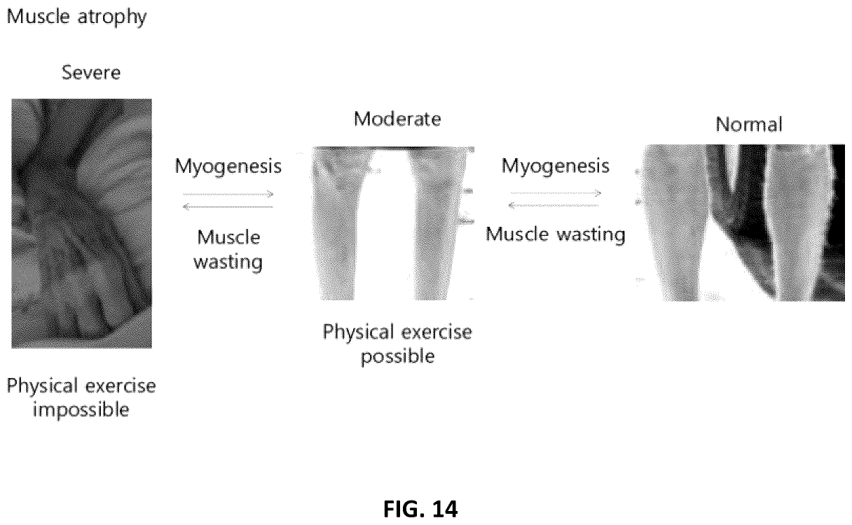

FIG. 14 illustrates the progression of muscle atrophy, which is one aspect of cancer cachexia (the other aspect being cancer). As used herein, “muscle atrophy” can refer to a disease in which muscles of the body (e.g., legs and arms) are gradually atrophied, sometimes symmetrically. Muscle atrophy may accompany the occurrence of cancer, aging, renal diseases, genetic diseases, and various chronic diseases. Muscle atrophy is represented by amyotrophic lateral sclerosis (Lou Gehrig's disease), spinal progressive muscular atrophy, etc. A normal or healthy individual (far right) is capable of exercise. Muscle wasting or atrophy can lead to moderate muscular atrophy, where physical exercise is still possible. Moderate muscle atrophy can be characterized by reduced motor function, wherein the patient can still walk, although not as well as a healthy individual (e.g., a patient with moderate muscle atrophy can have an abnormal gait, slouch, and/or have a rigid posture, etc.). Further muscle wasting can lead to severe muscle atrophy, where physical exercise is no longer possible. Severe muscle atrophy can be characterized by reduced or complete loss of motor function, such as an inability to walk. Generally, the stage of muscle atrophy can be determined or classified by muscle volume and/or athletic ability. Likewise, myogenesis can restore muscle function. The methods and systems of the present disclosure can promote myogenesis in patients suffering from severe and moderate muscle atrophy to restore normal muscle function. For patients suffering from muscle atrophy, methods and system of the present disclosure can be helpful in promoting myogenesis such that the muscle atrophy is reduced, and the patient is able to tolerate a cancer treatment that may not otherwise be possible, for example, in a patient suffering from severe muscle atrophy.

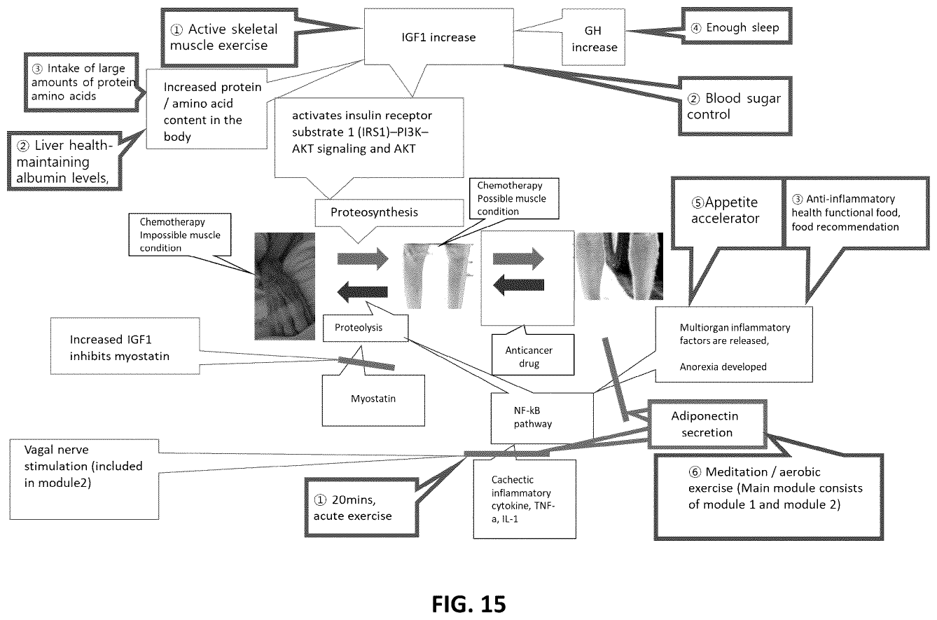

FIG. 15 illustrates various exemplary biochemical pathways and physiological symptoms associated with cancer cachexia, as well as exemplary applications of certain embodiments of the present disclosure and associated effects. Cachexia can be characterized by an acquired, accelerated loss of muscle caused by an underlying disease. When cachexia is seen in a patient with cancer, the condition may be referred to as “cancer cachexia”. Cancer cachexia affects the majority of patients with advanced cancer and is associated with a reduction in treatment tolerance, response to therapy, quality of life and duration of survival. Cancer cachexia is a multifactorial syndrome characterized by an ongoing loss of skeletal muscle mass, with or without loss of fat mass, which cannot be fully reversed by conventional nutritional support and leads to progressive functional impairment. Skeletal muscle loss may be the most significant event in cancer cachexia.

As mentioned above, one aim of the methods and systems of the present disclosure is to at least partially reduce muscle atrophy such that a patient is able to tolerate cancer treatment. Muscle atrophy can be caused by increased myostatin levels, which promote proteolysis. In one aspect, methods and systems of the present disclosure increase insulin-like growth factor 1 (IGF1) and/or a splice variant of IGF-1 called mechano growth factor (MGF) levels (IGF1/MGF level), for example, using voluntary skeletal muscle exercise modules and/or digital instructions for a patient's diet, which can (i) inhibit myostatin and prevent muscular atrophy associated with proteolysis, and/or (ii) activate insulin receptor substrate 1 (IRS1)-PI3K-AKT signaling and AKT to promote proteosynthesis. Muscle atrophy can also result from activation of the NF-κB pathway (e.g., via release of multiorgan inflammatory factors and/or cachetic inflammatory cytokines TNF-α/IL-1), which promote proteolysis. In one aspect, methods and systems of the present disclosure inhibit activation of the NF-κB pathway by promoting adiponectin secretion, for example, through meditation and/or aerobic exercise. In another aspect, methods and systems of the present disclosure inhibit activation of the NF-κB pathway by inhibiting cachetic inflammatory cytokines TNF-α/IL-1 through vagal nerve stimulation.

In addition to cachexia, cancer is another aspect of cancer cachexia. For early stage cancer patients, stimulating IGF1 secretion, for example, by physical exercise, may help treat cancer cachexia. In patients with terminal cancer, however, the above strategy may have side effects that promote HIF1 and accelerate cancer progression. Thus, for a late stage cancer, cancer cachexia therapy may exclude treatments to stimulate IGF1 secretion, such as a physical exercise. Accordingly, the prescription of module assigned to each patient can be different according to the progress of cancer. Thus, the early external activity for the early state cancer patients may be different from the late external activity for the late stage cancer patients as described herein.

In some embodiments, the late external activity includes touching a screen with a finger or a heel, and deep breathing. The touching may be monitored by a touch sensor, acceleration sensor, and/or gyro sensor. In additional embodiments, the late external activity includes lifting a head while lying down, and raising a hand or leg while lying down, which may be monitored by acceleration sensor, gyro sensor, or a touch sensor. In further embodiments, the late external activity includes grabbing phone while lying down, turning a head while lying down. The grabbing may be monitored by a touch sensor or a gesture recognition. The head turning may be monitored by a face recognition.

A digital apparatus and an application for inhibiting progression of and treating cancer cachexia according to the present disclosure will be described below.

Generally speaking, disease therapy is carried out by analyzing a certain disease in terms of pathophysiological functions and dispositions in order to determine a start point, a progression point, and an end point for the disease. Also, an indication of the disease is defined by characterization of the corresponding disease and statistical analysis of the disease. Also, patient's physiological factors, especially biochemical factors, which correspond to the verified indications, are analyzed, and the patient's biochemical factors are restricted a narrow extent associated with the disease to deduce a mechanism of action.

Next, a therapeutic hypothesis, in which the corresponding disease is treated by controlling actions and environments directly associated with regulation of the corresponding biochemical factors associated with the disease, is deduced. To realize this therapeutic hypothesis into digital therapeutics, a digital therapeutic hypothesis for achieving a therapeutic effect through repeated digital instruction and execution, which are associated with the “control of patient's action/environment→regulation of biochemical factors, is proposed. The digital therapeutic hypothesis of the present disclosure is realized as a digital apparatus and an application is realized as a digital apparatus and an application configured to present changes in patient's actions, and patient's participation in the form of specific instructions and collect and analyze execution of the specific instructions.

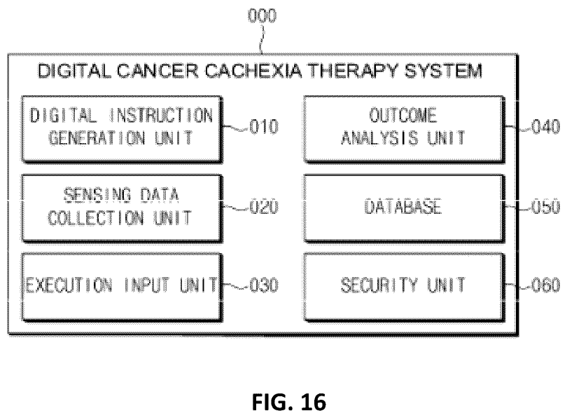

FIG. 16 is a block diagram showing a configuration of the digital apparatus for treating cancer cachexia according to one embodiment of the present disclosure. Referring to FIG. 16, a digital system 000 for treating cancer cachexia according to one embodiment of the present disclosure may include a digital instruction generation unit 010, a sensing data collection unit 020, an execution input unit 030, an outcome analysis unit 040, a database 050, and a security unit 060.

Based on the mechanism of action in and the therapeutic hypothesis and digital therapeutic hypothesis for cancer cachexia, a doctor (a second user) may prescribe digital therapeutics, which are realized in a digital apparatus and an application for treating cancer cachexia, for the corresponding patient. In this case, the digital instruction generation unit 010 is a device configured to provide a prescription of the digital therapeutics to a patient as a specific behavioral instruction that the patient may execute based on the interaction between the biochemical factors for cancer cachexia and the patient's behaviors. For example, the biochemical factors may include IGF1, HIF1, and the like, but the present disclosure is not limited thereto. For example, all types of biochemical factors that may cause cancer cachexia may be considered.

The digital instruction generation unit 010 may generate digital instructions based on the inputs from the doctor. In this case, the digital instruction generation unit 010 may generate digital instructions based on information collected by the doctor when diagnosing a patient. Also, the digital instruction generation unit 010 may generate digital instructions based on the information received from the patient. For example, the information received from the patient may include the patient's basal factors, medical information, and digital therapeutics literacy. In this case, the basal factors may include amount of the patient's activity, heart rates, sleep, meals (nutrition and calories), and the like. The medical information may include the patient's electronic medical record (EMR), family history, genetic vulnerability, genetic susceptibility, and the like. The digital therapeutics literacy may include the patient's accessibility and an acceptance posture to the digital therapy instructions and the apparatus, and the like.

The digital instruction generation unit 010 may reflect the mechanism of action in and the therapeutic hypothesis for cancer cachexia in order to utilize imaginary parameters and generate a digital module. In this case, the imaginary parameters may be deduced in term of the patient's behaviors.

The digital instruction generation unit 010 generates digital instructions particularly designed to allow a patient to have a therapeutic effect, and provides the instructions to the patient. For example, the digital instruction generation unit 010 may generate specific digital instructions in each of digital therapeutic modules.

The sensing data collection unit 020 and the execution input unit 030 may collect the patient's execution outcomes of the digital instructions provided at the digital instruction generation unit 010. Specifically, the sensing data collection unit 020 configured to sense the patient's adherence to the digital instructions and the execution input unit 030 configured to allow a patient to directly input the execution outcomes of the digital instructions are included, and thus serve to output the patient's execution outcomes of the digital instructions.

The outcome analysis unit 040 may collect the patient's behavior adherence or participation in predetermined periods and report the patient's behavior adherence or participation to external systems. Therefore, a doctor may continue to monitor an execution course of the digital instructions through the application even when a patient does not directly visit a hospital.

The database 050 may store the mechanism of action in cancer cachexia, the therapeutic hypothesis for cancer cachexia, the digital instructions provided to the user, and the user's execution outcome data. FIG. 16 shows that the database 050 is included in the digital apparatus 000 for treating cancer cachexia. However, the database 050 may be provided in an external server.

Meanwhile, a series of loops including inputting the digital instructions at the digital instruction generation unit 010, outputting the patient's execution outcomes of the digital instructions at the sensing data collection unit 020/execution input unit 030, and evaluating the execution outcomes at the outcome analysis unit 040 may be repeatedly executed several times. In this case, the digital instruction generation unit 010 may generate patient-customized digital instructions for this cycle by reflecting the patient's digital instructions provided in the previous cycle and output values, and the evaluation.

As described above, according to the digital therapy apparatus for inhibiting progression of and treating cancer cachexia according to the present disclosure, the cancer cachexia therapy whose reliability may be ensured is possible by deducing the mechanism of action in cancer cachexia and the therapeutic hypothesis and digital therapeutic hypothesis for cancer cachexia in consideration of the biochemical factors for cancer cachexia, presenting digital instructions for treating cancer cachexia based on the mechanism of action and the therapeutic hypotheses, and collecting and analyzing execution of specific instructions.

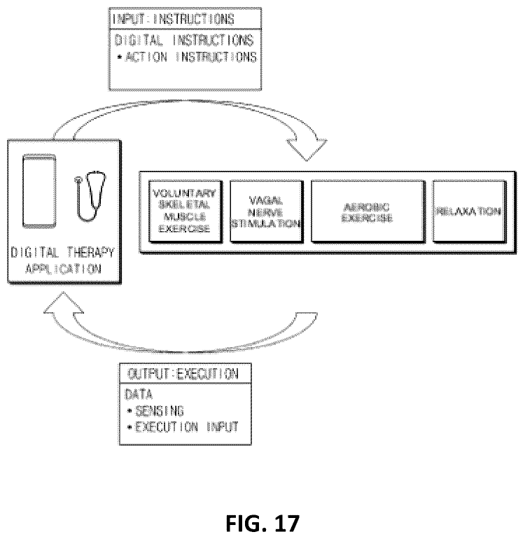

FIG. 17 is a diagram showing input and output loops of the digital application for treating cancer cachexia according to one embodiment of the present disclosure.

Referring to FIG. 17, the digital application for treating cancer cachexia according to one embodiment of the present disclosure may input the corresponding digital prescription for a patient in the form of instructions, and may output execution outcomes of the corresponding digital instructions.

The digital instructions provided to the patient may include specific action instructions for behaviors, and the like. As shown in FIG. 17, the digital instructions may include voluntary skeletal muscle exercise, vagal nerve stimulation, aerobic exercise, relaxation, and the like. However, the digital instructions are given by way of illustration only, and are not intended to be limiting to the digital instruction according to the present disclosure.

The patient's execution outcomes of the digital instructions consist of 1) log-in/log-out information for instructions and execution, 2) adherence information sensed as passive data such as voluntary skeletal muscle exercise, heart rates associated with the stress, a change in oxygen saturation, and the like, and 3) directly input information on the patient's execution outcomes.

The inhibition of the progression of and the treatment of cancer cachexia are shown to be achieved by repeatedly executing the aforementioned single feedback loop of FIG. 17 several times to regulate the biochemical factors.

Inhibitory and therapeutic effects on progression of the cancer cachexia may be more effectively achieved by gradual improvement of an instruction-execution cycle in the feedback loop, compared to the simply repeated instruction-execution cycle during the corresponding course of therapy.

For example, the digital instructions and the execution outcomes for the first cycle are given as input values and output values in a single loop, but new digital instructions may be generated by reflecting input values and output values generated in this loop using a feedback process of the loop to adjust the input for the next loop when the feedback loop is executed N times. This feedback loop may be repeated to deduce patient-customized digital instructions and maximize a therapeutic effect at the same time.

As such, in the digital apparatus and the application for treating cancer cachexia according to one embodiment of the present disclosure, the patient's digital instructions provided in the previous cycle (for example, a N−1^{st} cycle), and the data on instruction execution outcomes may be used to calculate the patient's digital instructions and execution outcomes in this cycle (for example, a N^{th} cycle). That is, the digital instructions in the next loop may be generated based on the patient's digital instructions and execution outcomes of the digital instructions calculated in the previous loop. In this case, various algorithms and statistical models may be used for the feedback process, when necessary.

As described above, in the digital apparatus and the application for treating cancer cachexia according to one embodiment of the present disclosure, it is possible to optimize the patient-customized digital instructions suitable for the patient through the rapid feedback loop.

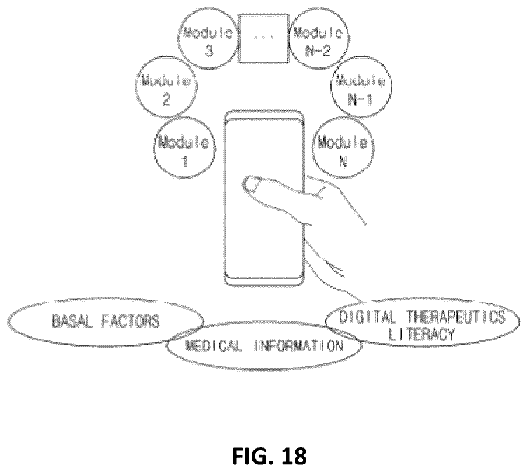

FIG. 18 is a diagram showing a background factors supporting the digital apparatus and the application for treating cancer cachexia according to one embodiment of the present disclosure

Referring to FIG. 18, the background factors may be considered together in the design of the modules in the digital apparatus and the application for treating cancer cachexia according to one embodiment of the present disclosure.

In this case, the background factors are elements necessary for correction of clinical trial outcomes during verification of the clinical effectiveness of digital cancer cachexia therapy according to the present disclosure. Specifically, in the background factors shown in FIG. 18, the basal factors may include activity, heart rates, sleep, meals (nutrition and calories), and the like, the medical information may include EMR, family history, genetic vulnerability, and susceptibility, and the like, which have been written when a patient visited a hospital, and the digital therapeutics literacy may include the patient's accessibility to the digital therapy instructions and the apparatus, and an acceptance posture.

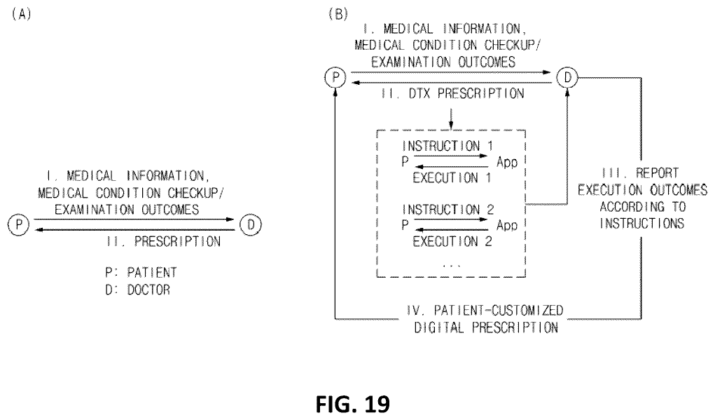

FIG. 19 is a diagram showing a method of assigning a patient-customized digital prescription using the digital apparatus and the application for treating cancer cachexia according to one embodiment of the present disclosure.

FIG. 19(A) show a prescription procedure for routine medical condition checkup of a patient by a doctor, and FIG. 19(B) show a method of allowing a doctor to assign a patient-customized digital prescription based on the analysis of a plurality of digital instructions and execution outcomes of the digital instructions.

In this way, when the digital apparatus and the application for treating cancer cachexia according to one embodiment of the present disclosure are used, the doctor may check the patient's instructions and execution outcomes for a given period and adjust the types of modules for treating cancer cachexia, and the instructions for each module in a patient-customized manner, as shown in FIG. 19(B).

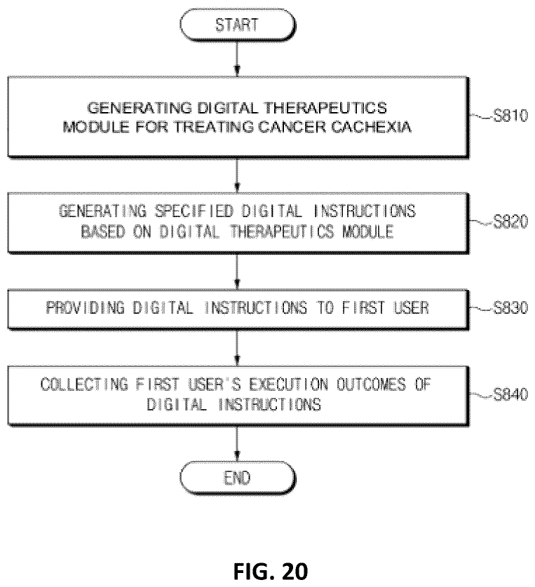

FIG. 20 is a flowchart illustrating operations in the digital application for treating cancer cachexia according to one embodiment of the present disclosure.

Referring to FIG. 20, the digital application for treating cancer cachexia according to one embodiment of the present disclosure may first generate a digital therapeutics module for treating cancer cachexia based on the mechanism of action in and the therapeutic hypothesis for cancer cachexia (S810). In this case, in S810, the digital therapeutics module may be generated based on the biochemical factors (for example, IGF1, HIF1, etc.) for cancer cachexia.

Meanwhile, in S810, the digital therapeutics module may be generated based on the inputs from the doctor. In this case, a digital therapeutics module may be generated based on the information collected by the doctor when diagnosing a patient, and the prescription outcomes recorded based on the information. Also, in S810, the digital therapeutics module may be generated based on the information (for example, basal factors, medical information, digital therapeutics literacy, etc.) received from the patient.

Next, in S820, specified digital instructions may be generated based on the digital therapeutics module. S820 may generate a digital therapeutics module by applying imaginary parameters about the patient's environments, behaviors, emotions, and cognition to the mechanism of action in and the therapeutic hypothesis for cancer cachexia.

In this case, the digital instructions may be generated for at least one of voluntary skeletal muscle exercise, vagal nerve stimulation, aerobic exercise, and relaxation modules. Then, the digital instructions may be provided to a patient (S830). In this case, the digital instructions may be provided in the form of digital instructions which are associated with behaviors and in which the patient's instruction adherence such as voluntary skeletal muscle exercise may be monitored using a sensor, or provided in the form of digital instructions in which a patient is allowed to directly input the execution outcomes.

After the patient executes the presented digital instructions, the patient's execution outcomes of the digital instructions may be collected (S840). In S840, the execution outcomes of the digital instructions may be collected by monitoring the patient's adherence to the digital instructions as described above, or allowing the patient to input the execution outcomes of the digital instructions.

In some embodiments, the patient or caregiver would input a sleep time, blood sugar level, food intake or meal information, appetite stimulant or other drug intake information, albumin levels, early external activity, late external activity, and/or internal activity information daily, and all or part of the information may be monitored by a doctor, for example, on his web.

Meanwhile, the digital application for treating cancer cachexia according to one embodiment of the present disclosure may repeatedly execute operations several times, wherein the operations include generating the digital instruction and collecting the patient's execution outcomes of the digital instructions. In this case, the generating of the digital instruction may include generating the patient's digital instructions for this cycle based on the patient's digital instructions provided in the previous cycle and the execution outcome data on the patient's collected digital instructions provided in the previous cycle.

As described above, according to the digital application for treating cancer cachexia according to one embodiment of the present disclosure, the reliability of the inhibition of progression of and treatment of cancer cachexia may be ensured by deducing the mechanism of action in and the therapeutic hypothesis for cancer cachexia in consideration of the biochemical factors for cancer cachexia, presenting the digital instructions to a patient based on the mechanism of action in and the therapeutic hypothesis for cancer cachexia, and collecting and analyzing the outcomes of the digital instructions.

Although the digital apparatus and the application for treating cancer cachexia according to one embodiment of the present disclosure have been described in terms of cancer cachexia therapy, the present disclosure is not limited thereto. For the other diseases other than the cancer cachexia, the digital therapy may be executed substantially in the same manner as described above.

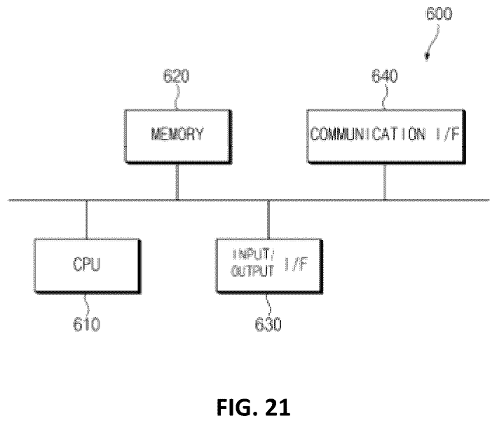

FIG. 21 is a diagram showing a hardware configuration of the digital apparatus for treating cancer cachexia according to one embodiment of the present disclosure.

Referring to FIG. 8, hardware 600 of the digital apparatus for treating cancer cachexia according to one embodiment of the present disclosure may include a CPU 610, a memory 620, an input/output I/F 630, and a communication I/F 640.

The CPU 610 may be a processor configured to execute a digital program for treating cancer cachexia stored in the memory 620, process various data for treating digital cancer cachexia and execute functions associated with the digital cancer cachexia therapy. That is, the CPU 610 may act to execute functions for each of the configurations shown in FIG. 16 by executing the digital program for treating cancer cachexia stored in the memory 620.

The memory 620 may have a digital program for treating cancer cachexia stored therein. Also, the memory 620 may include the data used for the digital cancer cachexia therapy included in the aforementioned database 050, for example, the patient's digital instructions and instruction execution outcomes, the patient's medical information, and the like.

A plurality of such memories 620 may be provided, when necessary. The memory 620 may be a volatile memory or a non-volatile memory. When the memory 620 is a volatile memory, RAM, DRAM, SRAM, and the like may be used as the memory 620. When the memory 620 is a non-volatile memory, ROM, PROM, EAROM, EPROM, EEPROM, a flash memory, and the like may be used as the memory 620. Examples of the memories 620 as listed above are given by way of illustration only, and are not intended to limit the present disclosure.

The input/output I/F 630 may provide an interface in which input apparatuses (not shown) such as a keyboard, a mouse, a touch panel, and the like, and output apparatuses such as a display (not shown), and the like may transmit and receive data (e.g., wirelessly or by hardline) to the CPU 610.

The communication I/F 640 is configured to transmit and receive various types of data to/from a server, and may be one of various apparatuses capable of supporting wire or wireless communication. For example, the types of data on the aforementioned digital behavior-based therapy may be received from a separately available external server through the communication I/F 640.

As described above, the computer program according to one embodiment of the present disclosure may be recorded in the memory 620 and processed at the CPU 610, for example, so that the computer program may be realized as a module configured to execute each of functional blocks shown in FIG. 3.

According to the digital apparatus and the application for treating, ameliorating, or preventing cancer cachexia according to the present disclosure, a reliable digital apparatus and application capable of inhibiting progression of and treating cancer cachexia may be provided by deducing a mechanism of action in cancer cachexia and a therapeutic hypothesis and a digital therapeutic hypothesis for cancer cachexia in consideration of biochemical factors for progression of cancer cachexia, presenting digital instructions to a patient, and collecting and analyzing execution outcomes of the digital instructions.

In certain embodiments, the present disclosure provides a method of treating, ameliorating, or preventing cancer cachexia in a subject in need thereof, the method comprising providing, by an electronic device to the subject, one or more first modules selected from the group consisting of a voluntary skeletal muscle exercise module, a vagal nerve stimulation module, an aerobic exercise module, and a relaxation module, each of the one or more first modules comprising one or more first instructions for the subject to follow, wherein the electronic device (i) comprises a sensor sensing adherence by the subject to the first instructions of the one or more first modules, (ii) transmits adherence information, based on the adherence, to a server, and (iii) receives one or more second instructions from the server based on the adherence information; and providing, by the electronic device to the subject, one or more second modules selected from the group consisting of a voluntary skeletal muscle exercise module, a vagal nerve stimulation module, an aerobic exercise module, and a relaxation module, the one or more second modules comprising the one or more second instructions.

In certain embodiments, the subject is diagnosed with cancer. In certain embodiments, the subject has cancer, and the subject is suffering from cancer cachexia. The subject may be a cancer patient, a patient at risk for cancer, or a patient with a family or personal history of cancer. In some cases, the patient is in a particular stage of cancer treatment. In certain embodiments, the subject has cancer, and the cancer is an early stage cancer. In certain embodiments, a subject with early stage cancer is a subject having a cancerous mass that is less than or equal to 0.1 centimeter (cm), less than or equal 0.5 cm, less than or equal 1 cm, less than or equal 2 cm, less than or equal 3 cm, less than or equal 4 cm, or less than or equal 5 cm in diameter. In certain embodiments, the subject has cancer, and the cancer is a late stage cancer. In certain embodiments, a subject with late stage cancer is a subject having a cancerous mass that is greater than or equal to 0.1 centimeter (cm), greater than or equal 0.5 cm, greater than or equal 1 cm, greater than or equal 2 cm, greater than or equal 3 cm, greater than or equal 4 cm, or greater than or equal 5 cm in diameter. Generally, the stage of cancer in a patient can be determined or classified based on HIF-1α expression levels (e.g., in the blood), hypoxia, and/or size of a cancerous mass in the subject.

Cancer patients may have any type of cancer. Examples of cancer can include, but are not limited to, adrenal cancer, anal cancer, basal cell carcinoma, bile duct cancer, bladder cancer, cancer of the blood, bone cancer, a brain tumor, breast cancer, bronchus cancer, cancer of the cardiovascular system, cervical cancer, colon cancer, colorectal cancer, cancer of the digestive system, cancer of the endocrine system, endometrial cancer, esophageal cancer, eye cancer, gallbladder cancer, a gastrointestinal tumor, kidney cancer, hematopoietic malignancy, laryngeal cancer, leukemia, liver cancer, lung cancer, lymphoma, melanoma, mesothelioma, cancer of the muscular system, Myelodysplastic Syndrome (MDS), myeloma, nasal cavity cancer, nasopharyngeal cancer, cancer of the nervous system, cancer of the lymphatic system, oral cancer, oropharyngeal cancer, osteosarcoma, Kaposi sarcoma, ovarian cancer, pancreatic cancer, penile cancer, pituitary tumors, prostate cancer, rectal cancer, renal pelvis cancer, cancer of the reproductive system, cancer of the respiratory system, sarcoma, salivary gland cancer, skeletal system cancer, skin cancer, small intestine cancer, stomach cancer, testicular cancer, throat cancer, thymus cancer, thyroid cancer, a tumor, cancer of the urinary system, uterine cancer, vaginal cancer, or vulvar cancer. The term ‘lymphoma’ may refer to any type of lymphoma including B-cell lymphoma (e.g., diffuse large B-cell lymphoma, follicular lymphoma, small lymphocytic lymphoma, mantle cell lymphoma, marginal zone B-cell lymphoma, Burkitt lymphoma, lymphoplasmacytic lymphoma, hairy cell leukemia, or primary central nervous system lymphoma) or a T-cell lymphoma (e.g., precursor T-lymphoblastic lymphoma, or peripheral T-cell lymphoma). The term ‘leukemia’ may refer to any type of leukemia including acute leukemia or chronic leukemia. Types of leukemia include acute myeloid leukemia, chronic myeloid leukemia, acute lymphocytic leukemia, acute undifferentiated leukemia, or chronic lymphocytic leukemia. In some cases, the cancer patient does not have a particular type of cancer. For example, in some instances, the patient may have a cancer that is not breast cancer.

Examples of cancer can include cancers that cause solid tumors as well as cancers that do not cause solid tumors. Furthermore, any of the cancers mentioned herein may be a primary cancer (e.g., a cancer that is named after the part of the body where it first started to grow) or a secondary or metastatic cancer (e.g., a cancer that has originated from another part of the body).

In some embodiments, the subject is at risk for cancer, and may be at risk because of a particular condition such as a pre-cancerous condition. Pre-cancerous conditions include but are not limited to actinic keratosis, Barrett's esophagus, atrophic gastritis, ductal carcinoma in situ, dyskeratosis congenita, sideropenic dysphagia, lichen planus, oral submucous fibrosis, solar elastosis, cervical dysplasia, leukoplakia, and erythroplakia). In some cases, a patient may be at risk of cancer because of cell or tissue dysplasia (e.g., an abnormal change in cell number, abnormal change in cell shape, abnormal change in cell size, or abnormal change in cell pigmentation).

In certain embodiments, the one or more first modules comprise the voluntary skeletal muscle exercise module. In certain embodiments, the voluntary skeletal muscle exercise module comprises one or more first instructions to increase MGF/IGF-1 secretion in the subject.

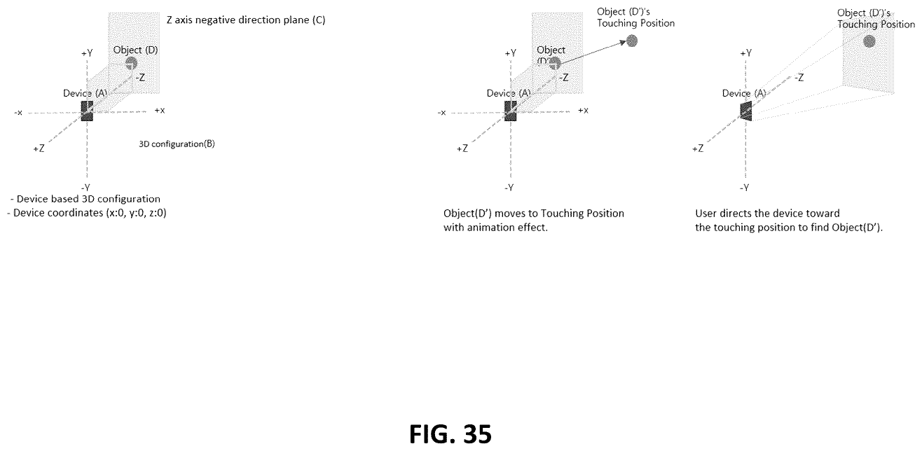

In certain embodiments, wherein the voluntary skeletal muscle exercise module comprises a screen touching exercise instruction to touch a randomly generated displayed animation on the electronic device. FIG. 35 is a diagram illustrating an exemplary screen touch sensing configuration for the voluntary skeletal muscle exercise module. The screen touch sensing configuration may be based on the electronic device's camera direction when running the digital application. The screen touch sensing configuration may be separated into the X-axis (left and right) and the Y-axis (up and down) (FIG. 35). In certain embodiments, the electronic device randomly generates a displayed animation of one object on the Z axis negative direction plane; When one object is touched, the selected object is removed, and a new object is created on the Z axis negative direction plane; with animation effects, the new object may appear on the screen of the electronic device for a brief period of time and moves to a new touching position; the electronic device may instruct the subject to move the device to a new location to find the new object and repeat the screen touching exercise. In certain embodiments, the screen touch sensing configuration may calculate the distance traveled by the device and record the data in the doctor portal and administrative portal (FIG. 35).

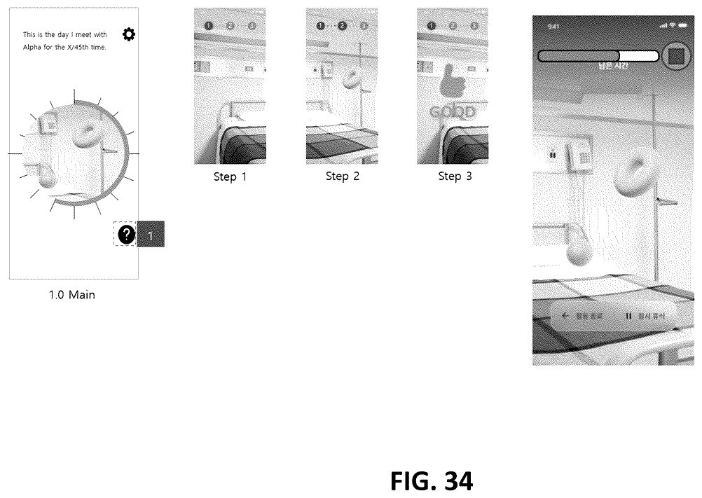

FIG. 34 is a diagram illustrating an exemplary usage flow of a digital application of the screen touching exercise of the present disclosure. At the start of the digital application, the subject may be instructed to perform screen touch exercises as an exemplary exercise of the voluntary skeletal muscle exercise modules. In some embodiments, the digital application may instruct the subject to pick up the electronic device and move the camera on the electronic device to find an animated object; when the animated object appears on the screen of the electronic device, the subject may be instructed to touch the animated object; When the subject touches the animated object, the tutorial ends. The digital application may instruct the subject to click the activity start button and activate a timer that measures the progress of the exercise module and the remaining time of the exercise module. The digital application may create coordinates dependent on the animated objects and their positions on the plane of the screen. The plane of the screen may be divided into four equal parts with maybe 1, 2, 3, or 4 animated objects displayed on different parts of the screen. The digital application may instruct the subject to touch the animated objects and repeat the exercise until the timer reaches 0.

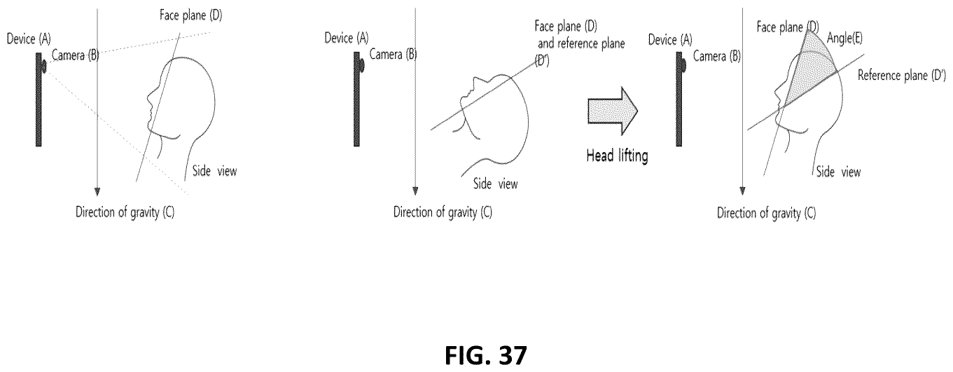

In certain embodiments, wherein the voluntary skeletal muscle exercise module comprises a head lifting exercise instruction to lift and position a head of the subject at a predetermined angle from the reference plane. FIG. 37 is a diagram illustrating an exemplary head lifting sensing configuration for the voluntary skeletal muscle exercise module. In certain embodiments, the electronic device recognizes the subject's face through the camera of the electronic device; The electronic device recognizes the direction of gravity and measures the plane position of the subject's face; The subject is instructed to set the position of the face plane as the reference plane before the exercise begins; The electronic device may instruct the subject to raise their head and measures the angle between the face plane and the reference plane; When the angle becomes greater than a certain value in the opposite direction to gravity, the electronic device determines that the subject has raised their head (FIG. 37).

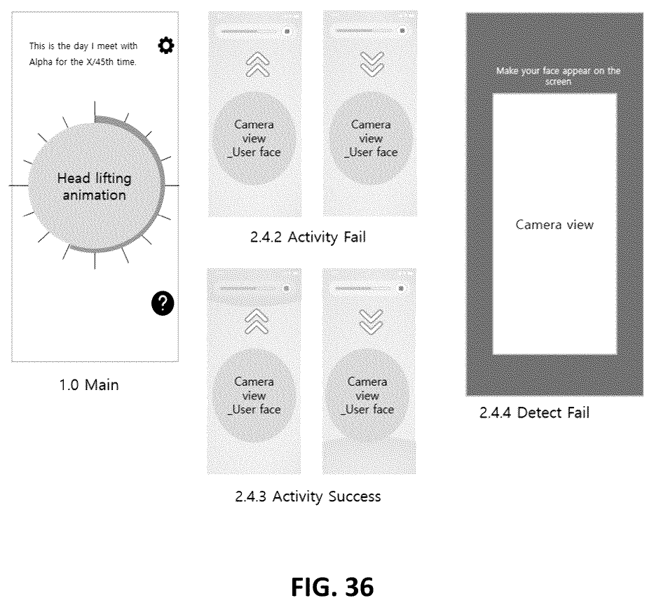

FIG. 36 is a diagram illustrating an exemplary usage flow of a digital application of the head lifting exercise of the present disclosure. At the start of the digital application, the subject may be instructed to perform head lifting exercises as an exemplary exercise of the voluntary skeletal muscle exercise modules. The digital application may instruct the subject to pick up the electronic device and position the electronic device on a cradle. The digital application may display a video of an animated head moving up and down and instruct the subject to follow the head motion of the animated head. The digital application may instruct the subject to click the activity start button and activate the timer that measures the progress of the exercise module and the remaining time of the exercise module. The digital application may display the subject's head on the electronic device screen and recognize the subject's head lifting motion; if the digital application does not recognize the subject's head lifting motion, the application may output a “please recognize your face” message. The digital application may end the activity when the timer reaches 0.

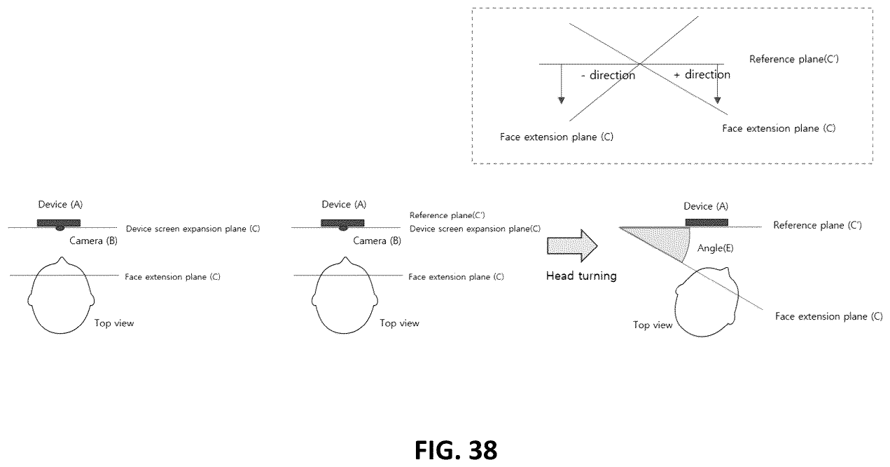

In certain embodiments, wherein the voluntary skeletal muscle exercise module comprises a head turning exercise instruction to turn a head of the subject clockwise and/or counterclockwise at a predetermined angle from the reference plane. FIG. 38 is a diagram illustrating the head turning sensing configuration for the voluntary skeletal muscle exercise module. In certain embodiments, the electronic device recognizes the subject's face through the camera of the electronic device; the electronic device sets the plane that extends the device screen, and measures the position of the plane of the subject's face; before the activity starts, the position of the extended plane of the device screen may be set as the reference plane; the electronic device may determine that the subject's face has rotated to the right when the subject's face plane moves more than a certain angle in the clockwise direction compared to the reference plane (FIG. 38). The electronic device may determine that the subject's face has rotated to the left when the subject's face plane moves more than a certain angle in the counterclockwise direction compared to the reference plane (FIG. 38).

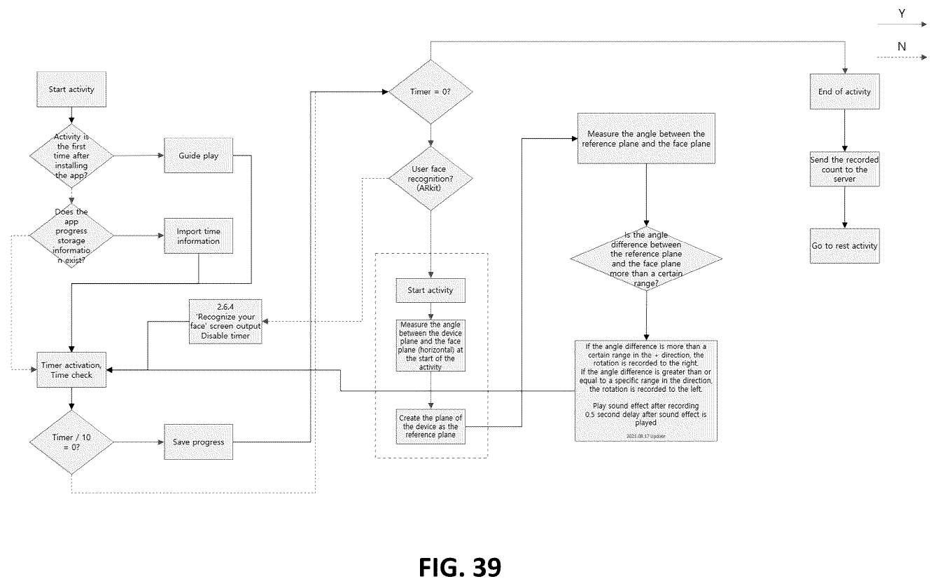

FIG. 39 is a flow chart illustrating an exemplary execution flow for the head turning instructions for the voluntary muscle exercise module. At the start of the digital application, the subject may be instructed to perform the head turning sensing configurations if this is the subject's first time installing the digital application. The digital application may load the application progress storage information if this is not the first time the subject has used the digital application. The digital application may import time and data information from previous stored data and activate a timer to save the subject's progress for the head turning exercise module. The digital application may also begin a user face recognition process before the start of the head turning activity by instructing the subject to perform the head turning sensing configurations. At the end of the head turning exercise module, the digital application may save the recorded data to the server and launch the rest module.

In certain embodiments, wherein the voluntary skeletal muscle exercise module comprises a hand gripping exercise instruction to grip the fist of the subject at a rate matching a displayed animation on the electronic device. In certain embodiments, the electronic device recognizes the subject's hand with the camera on the electronic device and saves the image every 0.01 seconds; the electronic device may measure the rate of the subject's open fist motion and closed fist motion compared to the displayed animations of an open fist and a closed fist on the electronic device; the electronic device may determine that the subject has performed the open fist and closed fist motion of the exercise when the photo stored for the open fist motion does not match the subject's current hand motion.

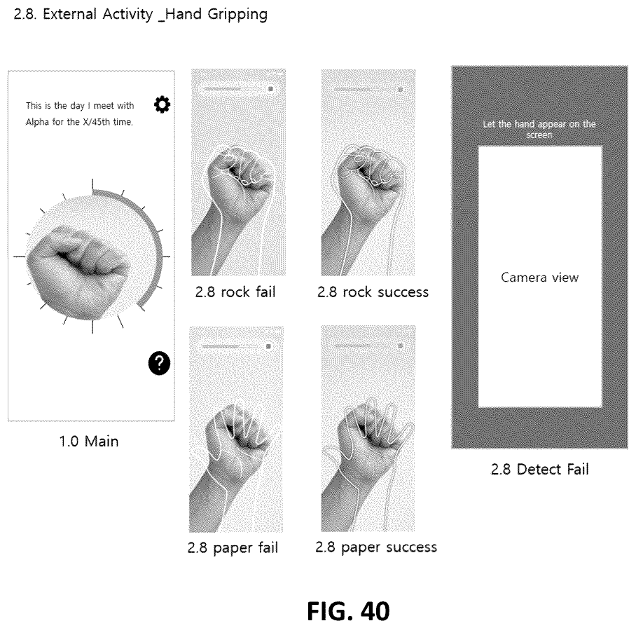

FIG. 40 is a diagram illustrating an exemplary usage flow of a digital application of the hand gripping exercise of the present disclosure. At the start of the digital application, the subject may be instructed to perform hand gripping exercises as an exemplary exercise of the voluntary skeletal muscle exercise modules. The digital application may instruct the subject to close their fists as the “rock” model animation and open their fists as the “paper” model animation while following the animated outline of a hand on the screen of the electronic device. The digital application may record the subject's hand gripping motions with the front facing camera of the electronic device. The digital application may instruct the subject to reposition their hands if the camera fails to detect the subject's hand. The digital application may end the activity when the timer reaches 0.

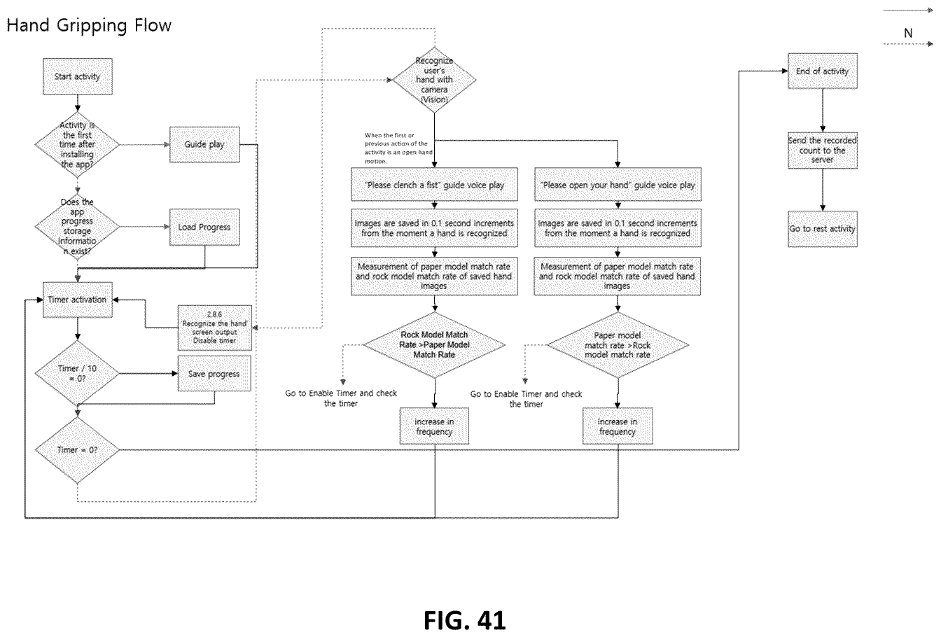

FIG. 41 is a flow chart illustrating an exemplary execution flow for the hand gripping instructions for the voluntary muscle exercise module. At the start of the digital application, the subject may be instructed to perform the hand gripping configurations if this is the subject's first time installing the digital application. The digital application may load the application progress storage information if this is not the first time the subject has used the digital application. The digital application may import time and data information from previous stored data and activate a timer to save the subject's progress for the hand gripping exercise module. The digital application may also begin a user hand recognition process before the start of the hand gripping exercise by instructing the subject to perform the hand gripping sensing configurations. The digital application may also recognize the subject's hand through the front facing camera of the electronic device and instruct the subject to “please clench a fist” or “please open your hand” using a guide voice. The digital application may save images in about 0.1 second increments from the moment the subject's hand is recognized. The digital application may measure the subject's rate of fist clenching with the saved images captured in about 0.1 second increments. The digital application may increase the frequency of the displayed animation instructing the subject to open and close their hand. At the end of the hand gripping exercise module, the digital application may save the recorded data to the server and launch the rest module.

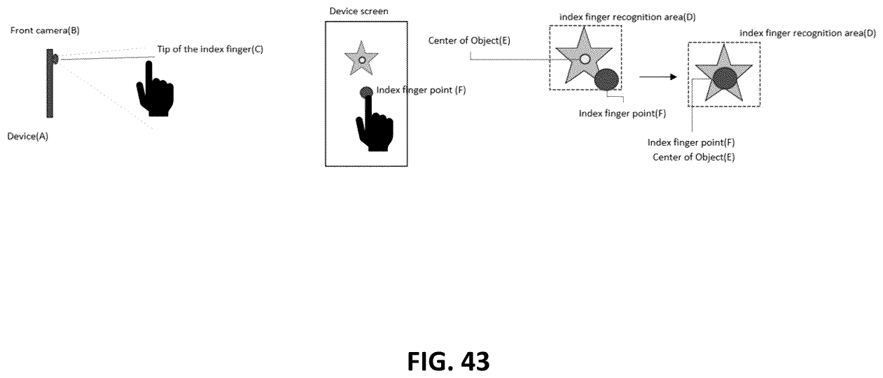

In certain embodiments, wherein the voluntary skeletal muscle exercise module comprises an arm shaking exercise instruction to move an object displayed on the electronic device across the screen with an index finger of the subject. FIG. 43 is a diagram illustrating an exemplary arm shaking exercise configuration for the voluntary skeletal muscle exercise module. In certain embodiments, the electronic device recognizes the subject's tip of the index finger with the front camera on the electronic device; On the screen of the electronic device, an object image in an arbitrary area, and a square-shaped index finger recognition area having a size that may include the object image are displayed; When the subject's index finger touches the index finger recognition area, the center of the displayed object image is moved to the position of the subject's index finger; As the subject moves their index finger, the object image follows on the screen of the electronic device; The electronic device measures the distance the object image has moved and sends the data to the doctor's web portal (FIG. 43).

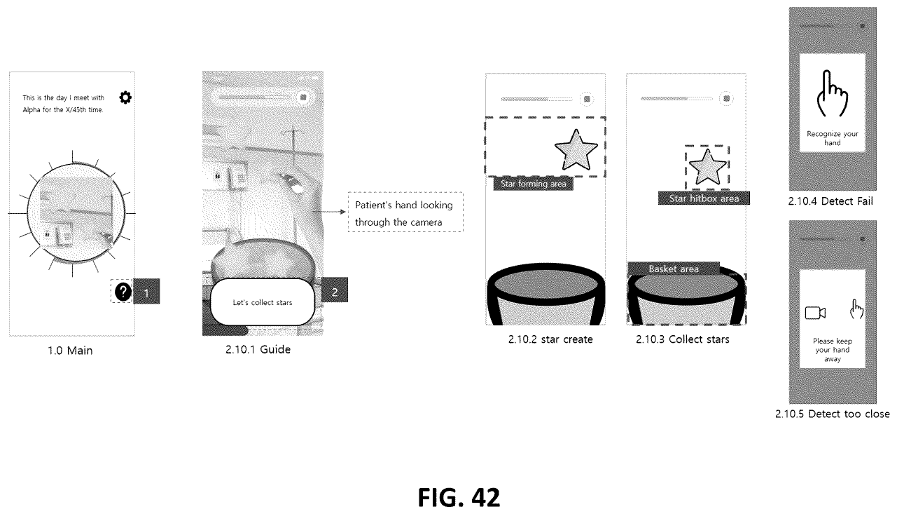

FIG. 42 is a diagram illustrating an exemplary usage flow of a digital application of the arm shaking exercise of the present disclosure. At the start of the digital application, the subject may be instructed to perform arm shaking exercises as an exemplary exercise of the voluntary skeletal muscle exercise modules. The digital application may instruct the subject to use their index fingers to move animated objects across the screen of the electronic device. The digital application may record the subject's arm shaking motions with the front facing camera of the electronic device. The digital application may instruct the subject to reposition their hands if the camera fails to detect the subject's hand. The digital application may end the activity when the timer reaches 0.

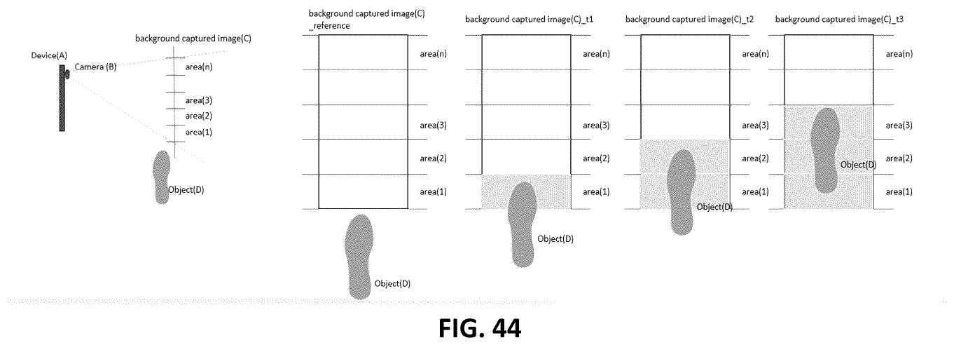

In certain embodiments, wherein the voluntary skeletal muscle exercise module comprises a leg lifting exercise instruct to lift their legs as measured by a change in a background area captured by the electronic device. FIG. 44 is a diagram illustrating an exemplary leg lifting exercise configuration for the voluntary skeletal muscle exercise module. In certain embodiments, the electronic device captures the background image of the subject and divide the image into multiple areas; the electronic device assigns an RGB (red, green, blue) value to each area and sets the background image as a reference value (FIG. 44). The electronic device determines that the subject has placed their foot in the corresponding area when the average RGB values of various areas of the image photographed at a specific time is different compared to the reference value of the background image (FIG. 44). The electronic device measures the movement of the subject when the amount of change in a specific area is sequentially greater than the reference value of the background image.

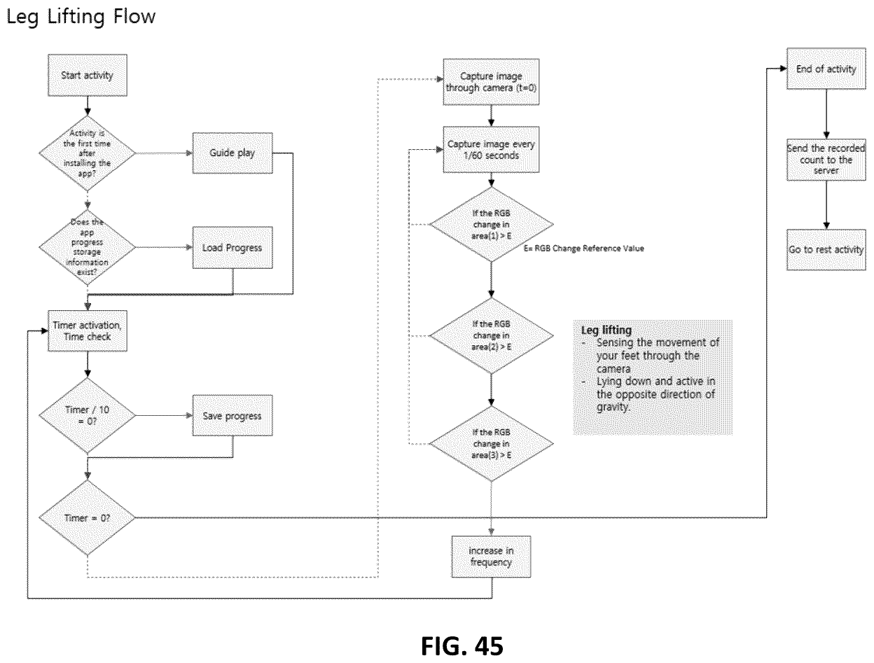

FIG. 45 is a flow chart illustrating an exemplary execution flow for the leg lifting exercise instructions for the voluntary muscle exercise module. At the start of the digital application, the subject may be instructed to perform the leg lifting exercise configurations if this is the subject's first time installing the digital application. The digital application may load the application progress storage information if this is not the first time the subject has used the digital application. The digital application may import time and data information from previous stored data and activate a timer to save the subject's progress for the leg lifting exercise module. The digital application may also begin a leg lifting recognition process before the start of the leg lifting exercise by instructing the subject to perform the leg lifting sensing configurations. The digital application may also recognize the subject's leg through the front facing camera of the electronic device and assign reference RGB values to the subject's background image. The digital application may save images in about 0.1 second increments from the moment the subject's leg is recognized. The digital application may measure the subject's rate of leg lifting with the saved images captured in about 0.1 second increments. The digital application may increase the frequency of the displayed animation instructing the subject to lift their leg if the change in RGB values is greater than reference RGB values. At the end of the leg lifting exercise module, the digital application may save the recorded data to the server and launch the rest module.

In certain embodiments, the one or more first instructions comprise instructions for aerobic, resistance, and/or concurrent exercise. Physical exercise (e.g., cardiovascular exercise) can promote IGF-1 production, which can (i) inhibit myostatin and prevent muscular atrophy associated with proteolysis, and/or (ii) activate insulin receptor substrate 1 (IRS1)-PI3K-AKT signaling and AKT to promote proteosynthesis. A module comprising physical exercise can include, for example, 20 minutes of high intensity exercise and 40 minutes of rest or low intensity exercise in a patient-bearable situation (for example, a module that mimics a low mountain climb, walk, etc.). This module can be repeated daily, and the effects observed after 1 week, 2 weeks, 3 weeks, 1 month, 2 months, 3 months, 4 months, 5 months, 6 months, 9 months, or 1 year.

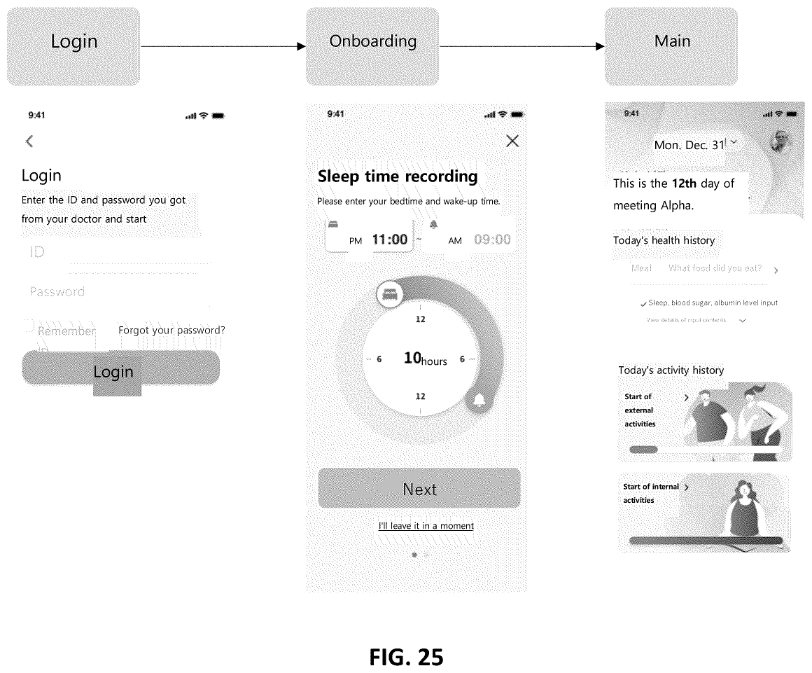

In certain embodiments, the one or more first modules comprise of the subject inputting health information into the electronic device. FIG. 25 is a diagram illustrating an exemplary patient portal structure of a digital application of the onboarding process of the present disclosure. In certain embodiments, the health information may comprise of the subject's sleep schedule, blood sugar levels, protein, amino acid, anti-inflammatory health food intake information, appetite stimulant consumption information, and albumin levels. In certain embodiments, the subject may input the health information as an onboarding process (FIG. 25). In certain embodiments, the digital application may instruct the subject to input their stable heart rate measurement. In certain embodiments, a subject's stable heart rate measurement may be a subject's resting heart rate measured at the initial onboarding process. In certain embodiments, the health information may be compiled into a daily checklist by the digital application wherein, the subject may be instructed to input their sleep and wake up times, blood sugar values, daily food intake, albumin levels, performance of daily external exercises, and performance of internal activities. In certain embodiments, the digital application may instruct the subject to input sleep and wake up times to measure whether the subject has enough sleep. In certain embodiments, a subject may be instructed to input blood sugar levels on an empty stomach before breakfast. In certain embodiments, the digital application may instruct the subject to input daily food intake to measure whether the subject is consuming enough protein, anti-inflammatory health food, and appetite stimulant. In certain embodiments, the digital application may instruct the subject to input albumin levels. In certain embodiments, the normal range of the subject's albumin levels may be about 3.5-5.2 g/dL. In certain embodiments, external activities may comprise of voluntary skeletal muscle exercises or aerobic exercises. In certain embodiments, internal activities may comprise of mediation exercises, breathing exercises, or massage exercises.

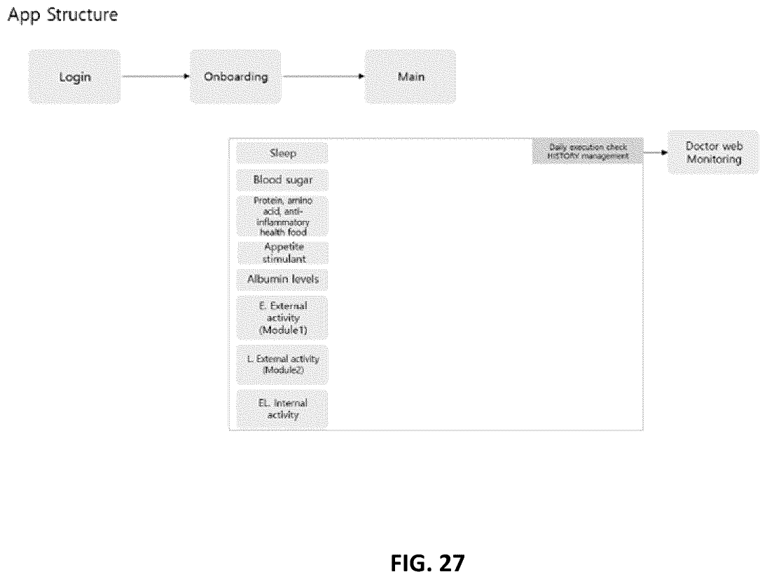

FIG. 27 is a flow chart illustrating an exemplary patient portal structure of the present disclosure. At the start of the application, the subject may be instructed to login with a “subject information key” automatically generated to subjects by healthcare providers. The health care provider may be instructed to input patient information before the subject's onboarding process. In addition, the subject may be instructed to onboard the application by inputting sleep and wake schedules, food intake information, and resting heart rate. In addition, the application records the subject's sleep and wake schedule, blood sugar, food intake information, albumin levels, and may instruct the subject to perform aerobic exercises, voluntary skeletal muscle exercises, and relaxation exercises.

In certain embodiments, the one or more first modules comprise the vagal nerve stimulation module, and the vagal nerve stimulation module comprises one or more first instructions to reduce inflammation in the subject. In certain embodiments, the one or more first modules comprise the vagal nerve stimulation module, and the vagal nerve stimulation module comprises at least one instruction selected from the group consisting of sense stimulation instructions for sight, sound, touch, taste, and smell. In certain embodiments, the vagal nerve stimulation module comprises one or more sense stimulation instructions for sight. In certain embodiments, the one or more sense stimulation instructions for sight include one or more instructions to view one or more figures or photos to stimulate autonomic nervous system. In certain embodiments, the vagal nerve stimulation module comprises one or more sense stimulation instructions for sound. In certain embodiments, the one or more sense stimulation instructions for sound include one or more instructions to hear one or more sounds to cause horror or relaxation. In certain embodiments, the vagal nerve stimulation module comprises one or more sense stimulation instructions for touch. In certain embodiments, the one or more sense stimulation instructions for touch include one or more instructions for abdominal breathing, controlling rate of breathing, cold massage, coughing, and skin massage. In some embodiments, the cold massage includes decreasing a temperature of a face of the subject. In certain embodiments, the vagal nerve stimulation module comprises one or more sense stimulation instructions for touch. In certain embodiments, the one or more sense stimulation instructions for touch include one or more instructions for abdominal breathing. In certain embodiments, the vagal nerve stimulation module comprises one or more sense stimulation instructions for taste. In certain embodiments, the one or more sense stimulation instructions for taste include one or more instructions to eat food to stimulate digestive glands in the subject. In certain embodiments, the vagal nerve stimulation module comprises one or more sense stimulation instructions for smell. In certain embodiments, the one or more sense stimulation instructions for smell include one or more instructions to stimulate digestive glands and/or to relax.

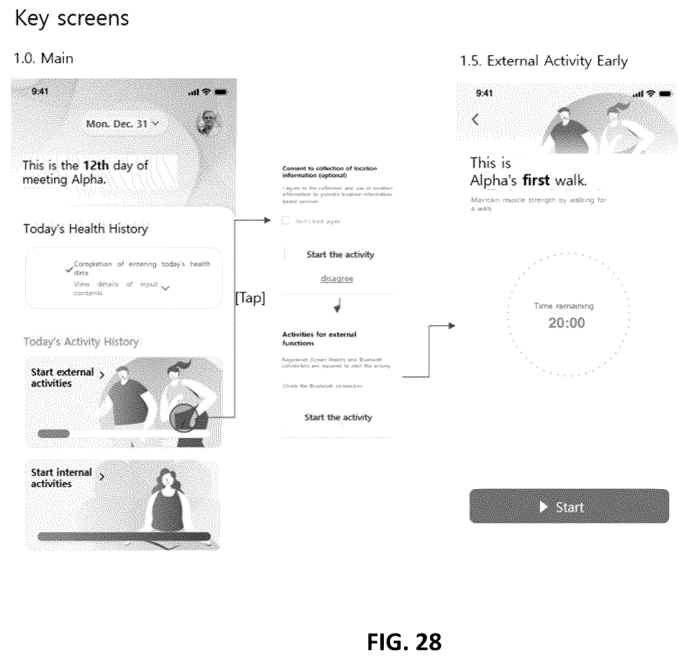

In certain embodiments, the one or more first modules comprise the aerobic exercise module, and the aerobic exercise module comprises one or more first instructions to increase adiponectin secretion in the subject. In certain embodiments, the one or more first instructions comprise one or more instructions for walking, biking, aerobic dance and/or swimming. FIG. 28 is a diagram illustrating an exemplary digital application structure that instructs the start of an external activity comprise, for example, aerobic exercises in about 30, 20, 10, or 5 minutes.

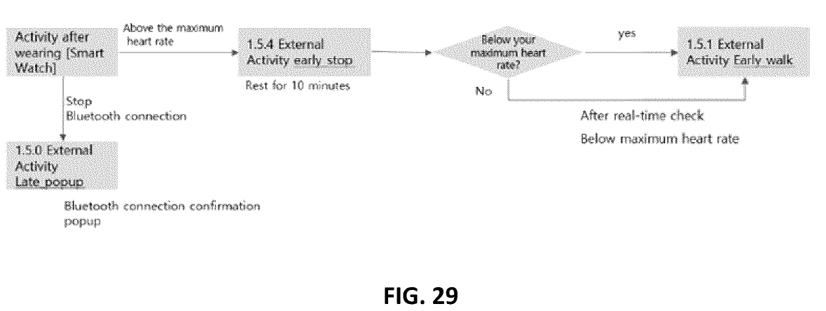

In certain embodiments, the one or more first modules may comprise of a guide screen that indicates the subject needs rest when maximum heart rate is reached. FIG. 29 is a flow chart illustrating the target heart rate calculation formula of a digital application of the present disclosure. In certain embodiments, the maximum heart rate may be calculated as [{207−(0.7*age)}-basal heart rate]*0.7+basal heart rate by the electronic device (FIG. 29). In certain embodiments, basal heart rate may be the subject's stable heart rate when setting up the device for the first time. The target heartrate of the subject may be kept within a medium intensity range of 50-70%. If the subject exceeds the maximum heart rate, the digital application may instruct the subject to stop the aerobic or voluntary skeletal muscle exercises (FIG. 16). In certain embodiments, the electronic device detects abnormal heart rate from the subject and a guide screen indicates that the subject should sufficiently rest. In certain embodiments, sufficient rest may be about 30, 20, 10 or 5 minutes, or whenever the subject's heart rate falls below the maximum heart rate. In certain embodiments, once the subject's heart rate falls below the maximum heart rate, the digital application may instruct the subject to resume the aerobic or voluntary muscle exercises (FIG. 29). In certain embodiments, after the subject's heart rate falls below the maximum heart rate, the subject may choose to end the aerobic exercise module or add one more aerobic exercise module. In certain embodiments, a guide screen instructing the subject to rest may be presented after the completion of the aerobic exercise module. In certain embodiments, the aerobic exercise module for late-stage subjects may be scheduled for about 30, 20, 10, or 5 minutes per day for a total of about 90 days.

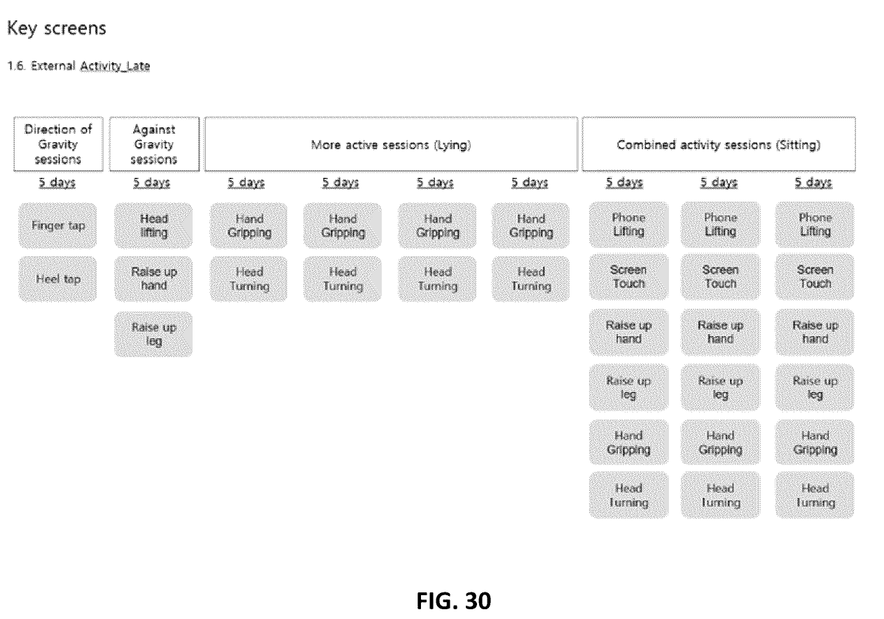

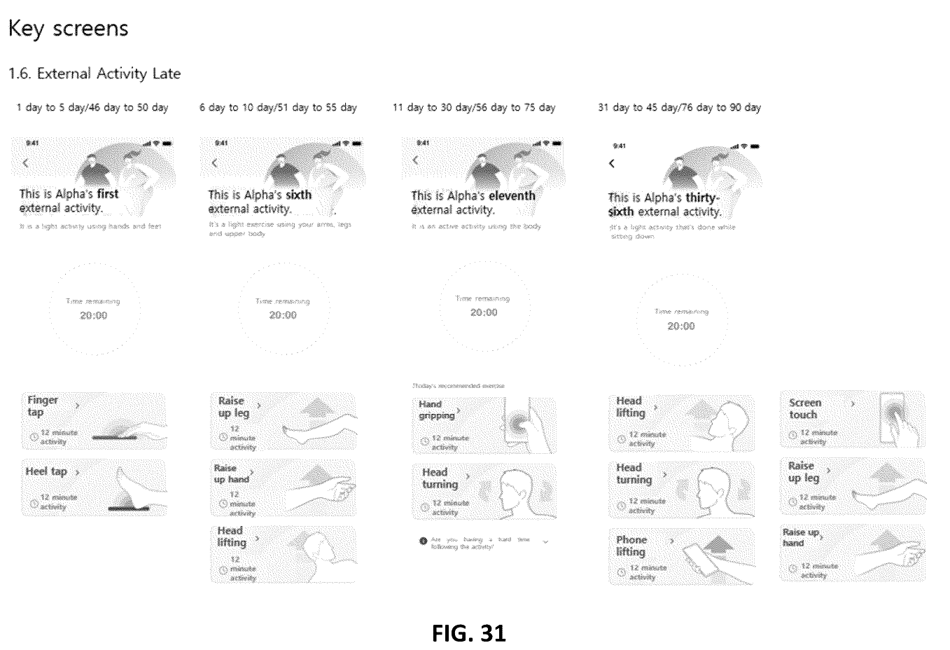

In certain embodiments, the one or more first modules comprise the voluntary skeletal muscle exercise module, and the voluntary skeletal muscle exercise module compromise one or more first instructions for gravity exercise sessions, anti-gravity exercise sessions, lying down exercise sessions, and sitting exercise sessions (FIG. 30). In certain embodiments, the gravity exercise sessions may comprise one or more instructions for finger taps and heel taps (FIG. 31). In certain embodiments, the finger taps exercise may instruct the subject to lie down and touch the electronic device screen with their finger for about 5, 4, 3, 2, or 1 minute, then rest for 3, 2, or 1 minute by deep breathing. In addition, the electronic device may provide sound feedback when the subject inputs the screen touch. In certain embodiments, the heel taps exercise may instruct the subject to lie down and touch the electronic device screen with their heels for about 5, 4, 3, 2, or 1 minute, then rest for 3, 2, or 1 minute by deep breathing. In addition, the electronic device may provide sound feedback when the subject inputs the heel screen touch.

In certain embodiments, one or more first instructions for anti-gravity exercise sessions comprise one or more instructions for head lifting, raising up hand, and raising up leg (FIG. 31). In certain embodiments, the head lifting exercise may instruct the subject to lie down and raise their head according to the guide instructions for about 5, 4, 3, 2, or 1 minute. In certain embodiments, the guide instructions may instruct the subject to raise their head for about 15, 10, 5, or 3 seconds and rest with deep breathing exercises for about 20, 15, 10, 8, or 6 seconds. In certain embodiments, the guide instructions for head lifting exercises may comprise voluntary breaks initiated by the subject at about 30, 20, 15, 10 and 5 seconds. In certain embodiments, the raising up hand exercise may instruct the subject to lie down and raise their left or right hand according to the guide instructions for about 5, 4, 3, 2, or 1 minute. In certain embodiments, the guide instructions may instruct the subject to raise their left or right hand for about 15, 10, 5, or 3 seconds and rest with deep breathing exercises for about 20, 15, 10, 8, or 6 seconds. In certain embodiments, the guide instructions for hand lifting exercises may comprise voluntary breaks initiated by the subject at about 30, 20, 15, 10 and 5 seconds. In certain embodiments, the raising up leg exercise may instruct the subject to lie down and raise their left or right leg according to the guide instructions for about 5, 4, 3, 2, or 1 minute. In certain embodiments, the guide instructions may instruct the subject to raise their left or right leg for about 15, 10, 5, or 3 seconds and rest with deep breathing exercises for about 20, 15, 10, 8, or 6 seconds. In certain embodiments, the guide instructions for raising up leg exercises may comprise voluntary breaks initiated by the subject at about 30, 20, 15, 10 and 5 seconds.

In certain embodiments, one or more first instructions for lying down exercise sessions may comprise one or more instructions for hand gripping, fist clenching, and head turning (FIG. 31). In certain embodiments, the hand gripping exercise may instruct the subject to lie down and grab the phone and press the screen for about 5, 4, 3, 2, or 1 second with their thumb and then rest by deep breathing for about 15, 10, 5, or 3 second s. In certain embodiments, after the subject has performed the hand gripping exercise for about 5, 4, 3, 2, or 1 minute, the subject may be instructed to rest for about 3, 2, or 1 minute by deep breathing. In certain embodiments, the fist clenching exercise may instruct the subject to lie down and slowly open and close their fists for about 15, 10, 5, or 3 seconds and then rest by deep breathing for about 15, 10, 5, or 3 seconds. In certain embodiments, after the subject has performed the hand gripping exercise for about 5, 4, 3, 2, or 1 minute, the subject may be instructed to rest for about 3, 2, or 1 minute by deep breathing. In certain embodiments, the head turning exercise may instruct the subject to lie down and slowly turn their head for about 15, 10, 5, or 3 seconds and then rest by deep breathing for about 15, 10, 5, or 3 seconds. In certain embodiments, after the subject has performed the head turning exercise for about 5, 4, 3, 2, or 1 minute, the subject may be instructed to rest for about 3, 2, or 1 minute by deep breathing.

In certain embodiments, one or more first instructions for sitting exercise sessions may comprise one or more instructions for phone lifting exercise, screen touching exercises, raising up hands exercise, raising up legs exercise, hand gripping exercise, and head turning exercise while the subject is sitting down (FIG. 31).

In certain embodiments, the voluntary skeletal muscle exercise module may be scheduled for about 60, 45, 30, or 15 days (FIG. 30). In certain embodiments, the voluntary skeletal muscle exercise module may be repeated and scheduled for a total of about 120, 90, 60, or 30 days (FIG. 30). In certain embodiments, one or more first instructions for gravity exercise sessions may be scheduled for about 15, 10, 5, or 3 days (FIG. 31). In certain embodiments, one or more first instructions for anti-gravity exercise sessions are scheduled for about 15, 10, 5, or 3 days (FIG. 30). In certain embodiments, one or more first instructions for lying down exercise sessions may be scheduled for about 30, 25, 20 or 15 days (FIG. 30). In certain embodiments, one or more first instructions for sitting down exercise sessions may be scheduled for about 20, 15, 10 or 5 days (FIG. 30). In certain embodiments, one or more instructions for gravity exercise sessions, anti-gravity exercise sessions, lying down exercise sessions, and sitting exercise sessions may be scheduled for about 30, 25, 20, 15, or 10 minutes per session each day (FIG. 31). In certain embodiments, the electronic device may display a guide screen indicating that the subject needs rest when maximum heart rate is reached.

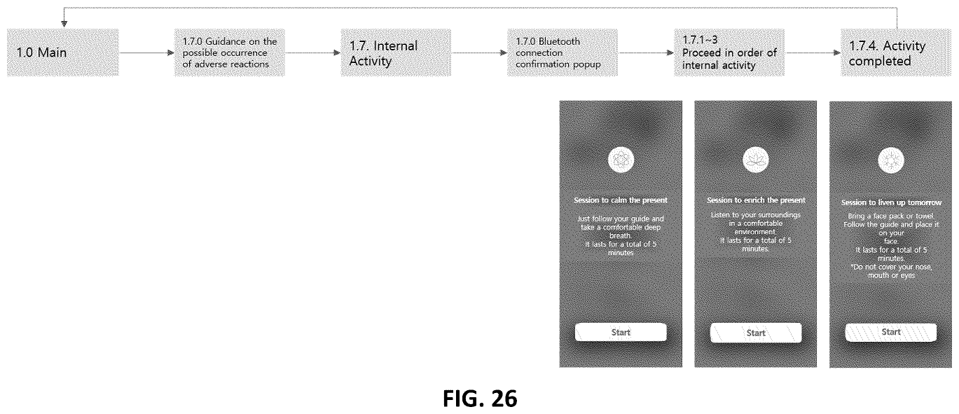

In certain embodiments, the one or more first modules comprise the relaxation module, and the relaxation module comprises one or more first instructions to increase adiponectin secretion in the subject. In certain embodiments, the one or more first instructions to increase adiponectin secretion in the subject comprise one or more meditation instructions. In certain embodiments, the one or more first instructions to increase adiponectin secretion in the subject comprise one or more sound instructions to hear relaxing sound. In some embodiments, the one or more sound instructions include generating relaxing sound, for example, including white noise. In additional embodiments, the device described herein comprises a speaker configured to generate the relaxing sound. One mechanism by which adiponectin can be increased is vagal nerve stimulation, which produces an anti-inflammatory effect and proteolysis block. In some embodiments, the relaxation module comprises meditation. In some embodiments, the relaxation module may instruct the subject to meditate by listening to white noise in a comfortable environment for about 10, 7, 5, or 3 minutes. In some embodiments, the relaxation module comprises deep breathing. In some embodiments, the relaxation module may instruct the subject to slowly breath in for about 10, 7, 5, or 3 seconds and exhale for 5, 3, or 1 second. In some embodiments, the relaxation module comprises massage exercises. In some embodiments, the relaxation module may instruct the subject to bring a face pack or towel and place it on the subject's face without covering the nose, mouth or eyes for about 10, 7, 5, or 3 minutes. In some embodiments, the relaxation module enables abdominal nerve stimulation. In some embodiments, the relaxation module is performed in an atmosphere or environment that is relaxing to the patient. In some embodiments, the relaxation module comprises listening to music. In some embodiments, the relaxation modules are scheduled for about 30, 20, 10 or 5 minutes. This module can be repeated daily, and the effects observed after 1 week, 2 weeks, 3 weeks, 1 month, 2 months, 3 months, 4 months, 5 months, 6 months, 9 months, or 1 year. FIG. 26 is a flow chart illustrating an exemplary internal activity use flow of a digital application of the meditation, breathing, and massage modules in the present disclosure. The subject may be instructed in the internal activity modules of the digital application to perform breathing exercises, mediation exercises, or massage exercises for a duration of about 30, 20, 10 or 5 minutes.

A session may comprise any number of digital therapeutic modules. In some embodiments, a session may comprise two or more digital therapeutic modules. In some embodiments, a session may comprise 3 or more, 4 or more, 5 or more, 6 or more, 7 or more, 8 or more, 9 or more, 10 or more, 11 or more, 12 or more, 13 or more, 14 or more, 15 or more, 20 or more, or 25 or more digital therapeutic modules. A session may comprise any number of digital therapeutic modules, and the digital therapeutic modules may be independently selected from a voluntary skeletal muscle exercise module, a vagal nerve stimulation module, an aerobic exercise module, and a relaxation module. In some embodiments, a session may consist of 4 digital therapeutic modules, and the digital therapeutic modules comprise a voluntary skeletal muscle exercise module, a vagal nerve stimulation module, an aerobic exercise module, and a relaxation module. A person of skill in the art will appreciate that there are a vast number of combinations for the number and type(s) of digital therapeutic modules that may go into a particular session. A session can be repeated as frequently or as infrequently as needed. In some embodiments, a session can be repeated 5 times per day, 4 times per day, 3 times per day, 2 times per day, daily, every 2 days, every 3 days, every 4 days, every 5 days, every 6 days, every 7 days, every 2 weeks, every 3 weeks, or every 4 weeks.

For patients having late stage cancer, it is contemplated that a voluntary skeletal muscle exercise module can cause the patient harm. Accordingly, in certain embodiments of the present disclosure, if a subject has late stage cancer, a voluntary skeletal muscle exercise module is excluded from the method or system for treating cancer cachexia. In certain embodiments of the present disclosure, if a subject has a cancerous mass that is that is greater than or equal to 0.1 centimeter (cm), greater than or equal 0.5 cm, greater than or equal 1 cm, greater than or equal 2 cm, greater than or equal 3 cm, greater than or equal 4 cm, or greater than or equal 5 cm in diameter, a voluntary skeletal muscle exercise module is excluded from the method or system for treating cancer cachexia.

In some embodiments, the subject has moderate muscle atrophy, and the one or more first modules consists of the voluntary skeletal muscle exercise module, the vagal nerve stimulation module, the aerobic exercise module, and the relaxation module. In some embodiments, the subject has moderate muscle atrophy, and the one or more first modules consists of the vagal nerve stimulation module, the aerobic exercise module, and the relaxation module.

In some embodiments, the subject has severe muscle atrophy and the method excludes providing a voluntary skeletal muscle exercise module and further excludes providing an aerobic exercise module.

In some embodiments, the external reviewer comprises a health professional (e.g., a healthcare provider or doctor). In some embodiments, the external reviewer comprises an artificial intelligence (AI). The term “artificial intelligence” can refer to intelligence exhibited by machines. In computer science, an ideal “intelligent” machine is a flexible rational agent that perceives its environment and takes actions that maximize its chance of success at some goal (e.g., treating cancer cachexia in a patient). Colloquially, the term “artificial intelligence” is applied when a machine mimics “cognitive” functions that humans associate with other human minds, such as “learning” and “problem solving. The term artificial intelligence may refer to an algorithm that may apply learning skills on multiple types of information (such as physiological information, additional information and person's medical history).

In some embodiments, the digital apparatus comprises a sensor, and the sensor comprises one or more of: a camera, an accelerometer, a magnetometer, a light sensor, a microphone, a proximity sensor, a touch sensor, a gyroscope, a Global Positioning System (GPS) sensor, an ambient light sensor, a fingerprint sensor, a pedometer, a heart rate sensor, a thermometer, a gesture recognition, and a face recognition. In some embodiments, the sensor comprises a touch sensor, and the subject provides the adherence information to the electronic device using the touch sensor.

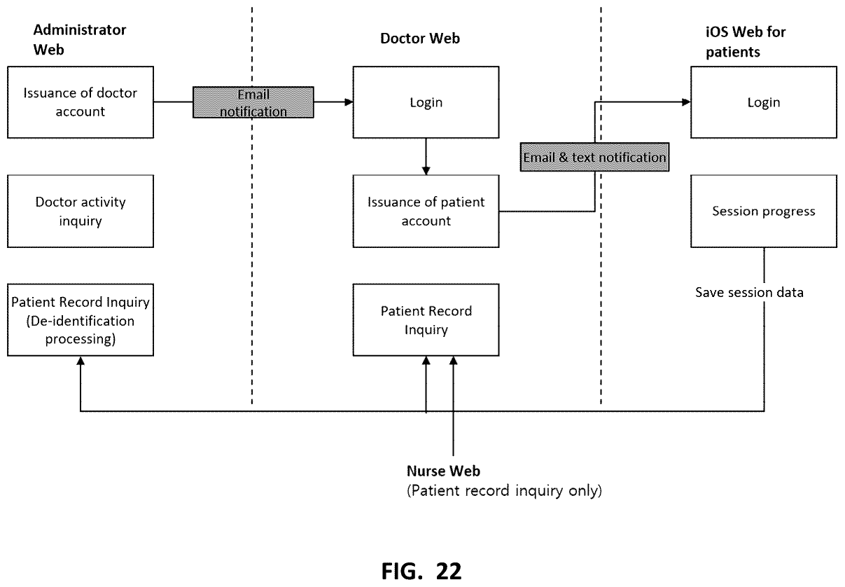

FIG. 22 depicts a flow chart illustrating a system for treating cancer cachexia, the system comprising an administrative portal (e.g., Administrator's web), a healthcare provider portal (e.g., Doctor's web) and a digital apparatus configured to execute a digital application (e.g., an application or ‘app’) for treating cancer cachexia in a subject. Among other things, the Administrator's portal allows an administrator to issue doctor accounts, review doctor information, and review de-identified patient information. Among other things, the Healthcare Provider's portal allows a healthcare provider (e.g., a doctor) to issue patient accounts, and review patient information (e.g., age, prescription information, and status for having completed one or more digital therapeutic modules or sessions). Among other things, the digital application allows a patent access to complete one or more digital therapeutic modules or sessions.

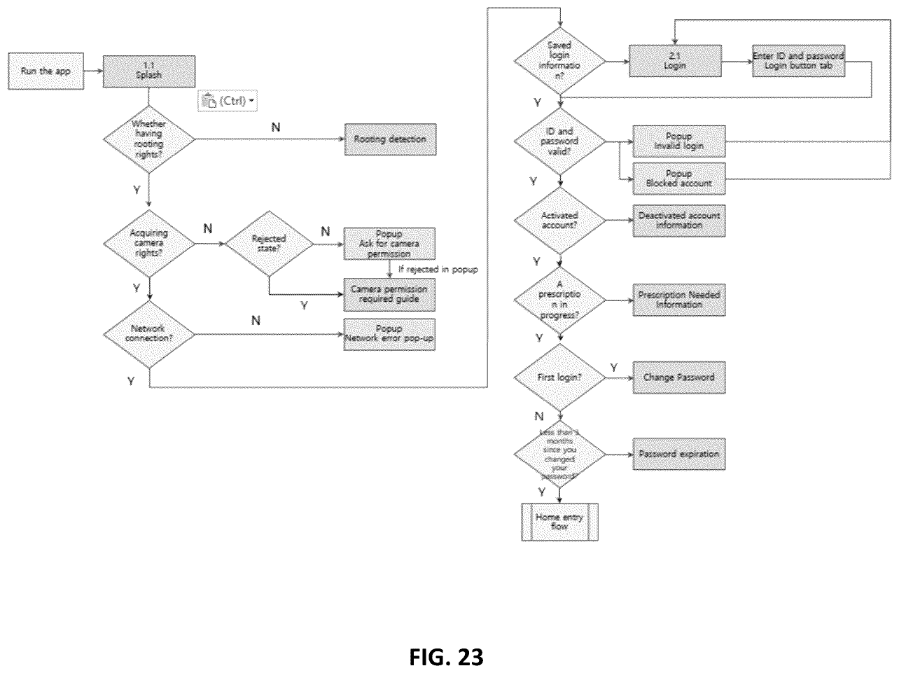

FIG. 23 is a flow chart illustrating an exemplary digital application usage flow of the present disclosure At the start of the application, a splash process may initiate the privacy and data access consent requests. The privacy and data access consent requests of the application may ask the subject to allow rooting rights, camera rights, and network connection rights on the electrical device. After consent is given to the application, the subject may be instructed to proceed with login verification. In addition, the login verification process may comprise, for example, the registration of ID and password, the activation of account, the confirmation of a prescription in progress, the option to change password if this is a first login, or the option to change password as a result of password expiration.

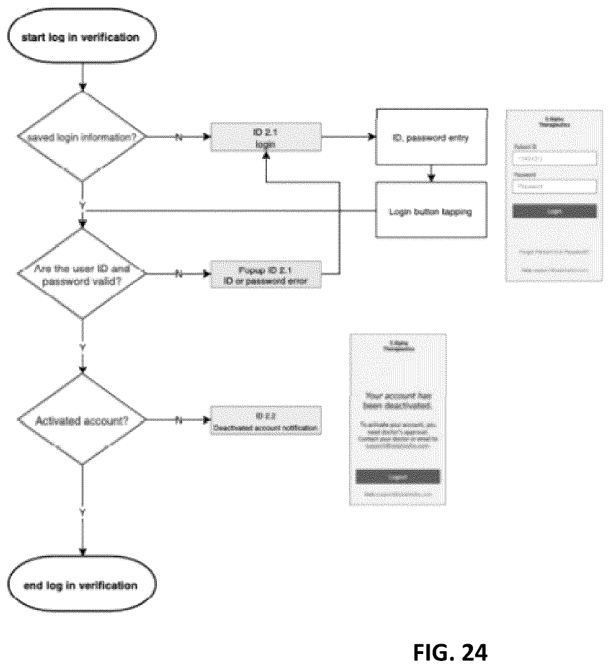

FIG. 24 depicts a flow chart illustrating an execution flow for login verification during a splash process at the starting of the digital application. In addition, a prescription verification process may comprise, for example, determining if the treatment period has expired, determining whether the subject has been recently (e.g., within the last hour) performed a voluntary skeletal muscle exercise), determining if, based on the prescription, the subject's sessions for the day have been completed (e.g., the subject is compliant with the prescription). In such instances, the digital apparatus may notify the subject that there are no sessions available to be completed.

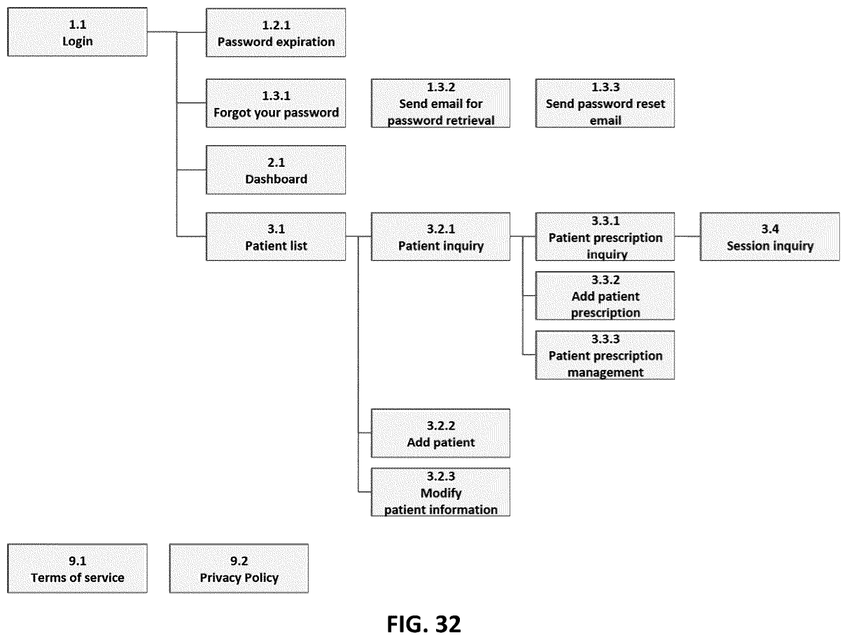

FIG. 32 is a flow chart illustrating an exemplary doctor portal structure and administrative portal structure of a digital application of the present disclosure. At the start of the digital application, the exemplary doctor portal may instruct the healthcare provider to login with a registered username and password. The digital application may instruct the healthcare provider to provide a new password if the healthcare provider's password has expired or the healthcare provider has forgotten the password. The digital application may provide a password retrieval process by sending an email to the healthcare provider. After the healthcare provider logs into the digital application, the dashboard of the digital application may provide a patient list, patient inquiries, patient prescription inquiries, and session inquiries. The digital application may allow the healthcare provider to add or modify patient information. The digital application may allow the healthcare provider to add patient prescription or manage patient prescriptions.

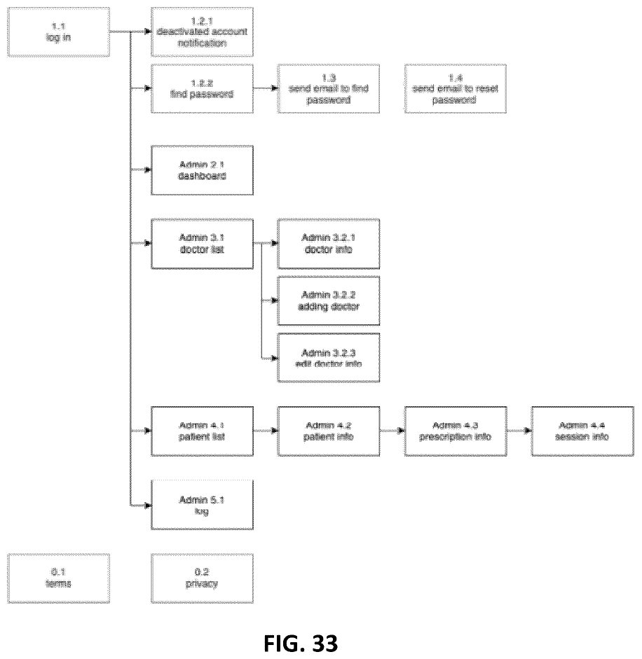

FIG. 33 is a flow chart illustrating an exemplary execution flow for an administrative portal in a system of the present disclosure. At the start of the digital application the exemplary administrative portal may instruct the administer to login with a registered username or password. The digital application may instruct the administrator to provide a password. If the administrator has forgotten the password, the digital application may provide a password retrieval process by sending an email to the administrator. After the administrator logs into the digital application, the dashboard of the digital application may provide a doctor list, patient list, and log of inputted data. The digital application may allow the administrator to review doctor information, add doctors to list of healthcare provider, and edit doctor information. The digital application may allow the administrator to review patient list, patient information, patient prescription information, and patient session information.

In some embodiments, the healthcare provider portal provides a healthcare provider with one or more options, and the one or more options provided to the healthcare provider are selected from the group consisting of adding or removing the subject, viewing or editing personal information for the subject, viewing adherence information for the subject, viewing a result of the subject for one or more at least partially completed digital therapeutic modules, prescribing one or more digital therapeutic modules to the subject, altering a prescription for one or more digital therapeutic modules, and communicating with the subject. In some embodiments, the one or more options comprise the viewing or editing personal information for the subject, and the personal information comprises one or more selected from the group consisting of an identification number for the subject, a name of the subject, a date of birth of the subject, an email of the subject, an email of the guardian of the subject, a contact phone number for the subject, a prescription for the subject, and one or more notes made by the healthcare provider about the subject. In some embodiments, the personal information comprises the prescription for the subject, and the prescription for the subject comprises one or more selected from the group consisting of a prescription identification number, a prescription type, a start date, a duration, a completion date, a number of scheduled or prescribed digital therapeutic modules to be performed by the subject, and a number of scheduled or prescribed digital therapeutic modules to be performed by the subject per day. In some embodiments, the one or more options comprise the viewing the adherence information, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules. In some embodiments, the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, and an indicator of whether the scheduled or prescribed digital therapeutic module was fully or partially completed.

A dashboard of a healthcare provider portal will be described in more detail. The dashboard of a healthcare provider portal may include the number of all patients associated with the present doctor's account. A graph may be used to show the number of patients who have opened the digital application for patient per day in the most recent 90 days. The number of patients in progress may also be viewed. A graph may be used to show the number of patients who have completed the daily sessions per day in the most recent 90 days. The healthcare provider portal may include a patient tab displaying a list of patients by displaying, for example, Patient ID (the unique identification number temporarily given to each patient when adding them on the list), Patient Name, Search bar for searching by ID, Name, Email, Memo, etc., and Add New Patient button for adding new patients. The patient tab may further display detailed information on a given patient, for example, detailed patient information, a button for editing patient information, prescription information, a button for adding a new prescription, a progress status for different each prescription, and a button or link for sending an email to the patient. The patient tab in the healthcare provider portal may further have an option for adding a new patient, for example, a button for adding a new patient, and displaying an error message when required patient information has not been provided. The patient tab in the healthcare provider portal may further include options for editing information of an existing patient by providing, e.g., a button or link for resetting a password, a button for deleting a given patient, and a button for saving changes. In addition, detailed prescription information can be displayed for a given patient. For example, the patient tab in the healthcare provider portal may include or display a button for editing prescription information, the duration of the sessions attended by the patient or subject, and an overview the treatment progress. Seven days are represented as a line or row of 7 squares. For 12 weeks, each 6 weeks may be presented separately. Different colors may be used to discern session statuses (e.g., grey for sessions not started, red for sessions not attended, yellow for sessions partially attended, and green for sessions fully attended). In some embodiments, the administrative portal provides an administrator with one or more options, and the one or more options provided to the administrator of the system are selected from the group consisting of adding or removing the healthcare provider, viewing or editing personal information for the healthcare provider, viewing or editing de-identified information of the subject, viewing adherence information for the subject, viewing a result of the subject for one or more at least partially completed digital therapeutic modules, and communicating with the healthcare provider. In some embodiments, the one or more options comprise the viewing or editing the personal information, and the personal information of the healthcare provider comprises one or more selected from the group consisting of an identification number for the healthcare provider, a name of the healthcare provider, an email of the healthcare provider, and a contact phone number for the healthcare provider. In some embodiments, the one or more options comprise the viewing or editing the de-identified information of the subject, and the de-identified information of the subject comprises one or more selected from the group consisting of an identification number for the subject, and the healthcare provider for the subject. In some embodiments, the one or more options comprise the viewing the adherence information for the subject, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules. In some embodiments, the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, and an indicator of whether the scheduled or prescribed digital therapeutic module was fully or partially completed.

A dashboard of an administrative portal may show the number of doctors. A graph may be used to show the number of doctors that have visited the digital application per day in the most recent 90 days. The number of all patients associated with the any doctor's account may be displayed on the administrative portal. A graph may be used to show the number of patients who have opened the digital application for patient per day in the most recent 90 days. The number of patients in progress may also be viewed. A graph may be used to show the number of patients who have completed the daily sessions per day in the most recent 90 days. A doctor tab in an administrative portal may display a list of doctors, by displaying, e.g., a search bar for searching for various doctors by name, email, etc., a button for adding a new doctor, the doctor's ID, a button for viewing detailed doctor information, and s deactivated doctor accounts. The doctor tab in the administrative portal further display a list of patients being cared for by a given doctor, with patient-identifying information redacted (*). For instance, the doctor tab displays is the doctor's account information, a button for editing the doctor's account information, a list of patients being cared for by the doctor, a list of patient ID numbers, a link or button for sending the doctor a registration email, a notification that the doctor's account has been deactivated, which only appears for deactivated accounts, and redacted or de-identified patient information. The doctor tab in the administrative portal may have options for adding a new doctor, editing information of an existing doctor, including activating or deactivating a doctor's account, etc. Additionally, a patient tab in the administrative portal displays information for one or more patients, wherein sensitive information is redacted. For example, the patient tab in the administrative portal displays detailed patient or prescription information for a given patient or detailed prescription information for a given patient.

While the disclosure has been shown and described with reference to certain exemplary embodiments thereof, it will be understood by those skilled in the art that various changes in form and details may be made therein without departing from the spirit and scope of the disclosure as defined by the appended claims.

### CERTAIN EMBODIMENTS

A method of treating cancer cachexia in a subject in need thereof, the method comprising: providing, by an electronic device to the subject, one or more first modules selected from the group consisting of a voluntary skeletal muscle exercise module, a vagal nerve stimulation module, an aerobic exercise module, and a relaxation module, each of the one or more first modules comprising one or more first instructions for the subject to follow; wherein the electronic device (i) comprises a sensor sensing adherence by the subject to the first instructions of the one or more first modules, (ii) transmits adherence information, based on the adherence, to a server, and (iii) receives one or more second instructions from the server based on the adherence information; and providing, by the electronic device to the subject, one or more second modules selected from the group consisting of a voluntary skeletal muscle exercise module, a vagal nerve stimulation module, an aerobic exercise module, and a relaxation module, the one or more second modules comprising the one or more second instructions.

The method according to Embodiment 1, wherein the one or more first modules comprise the voluntary skeletal muscle exercise module, and the voluntary skeletal muscle exercise module comprises one or more first instructions to increase MGF/IGF-1 secretion in the subject.

The method according to Embodiment 2, wherein the voluntary skeletal muscle exercise module comprises a screen touching exercise instruction to touch a randomly generated displayed animation on the electronic device.

The method according to Embodiment 2, wherein the voluntary skeletal muscle exercise module comprises a head lifting exercise instruction to lift and position a head of the subject at a predetermined angle from the reference plane.

The method according to Embodiment 2, wherein the voluntary skeletal muscle exercise module comprises a head turning exercise instruction to turn a head of the subject clockwise and/or counterclockwise at a predetermined angle from the reference plane.

The method according to Embodiment 2, wherein the voluntary skeletal muscle exercise module comprises a hand gripping exercise instruction to grip a fist of the subject at a rate matching a displayed animation on the electronic device.

The method according to Embodiment 2, wherein the voluntary skeletal muscle exercise module comprises an arm shaking exercise instruction to move an object displayed on the electronic device across the screen with an index finger of the subject.

The method according to Embodiment 2, wherein the voluntary skeletal muscle exercise module comprises a leg lifting exercise instruct to lift their legs as measured by a change in a background area captured by the electronic device.

The method according to Embodiment 2, wherein said one or more first instructions comprise instructions for aerobic, resistance, and/or concurrent exercise.

The method according to Embodiments 1-9, wherein the subject inputs health information into the electronic device.

The method according to Embodiment 10, wherein the health information comprises of the subject's sleep schedule, blood sugar levels, protein, amino acid, anti-inflammatory health food intake information, appetite stimulant consumption information, and albumin levels

The method according to any one of Embodiments 1-11, wherein the one or more first modules comprise the vagal nerve stimulation module, and the vagal nerve stimulation module comprises one or more first instructions to reduce inflammation in the subject.

The method according to any one of Embodiments 1-12, wherein the one or more first modules comprise the vagal nerve stimulation module, and the vagal nerve stimulation module comprises at least one instruction selected from the group consisting of sense stimulation instructions for sight, sound, touch, taste, and smell.

The method according to Embodiment 13, wherein the vagal nerve stimulation module comprises one or more sense stimulation instructions for sight, and the one or more sense stimulation instructions for sight include one or more instructions to view one or more figures to stimulate autonomic nervous system.

The method according to Embodiment 14, wherein the electronic device receives and displays the figures.

The method according to any one of Embodiments 13-15, wherein the vagal nerve stimulation module comprises one or more sense stimulation instructions for sound, and the one or more sense stimulation instructions for sound include one or more instructions to hear one or more sounds to cause horror or relaxation.

The method according to Embodiment 16, wherein the electronic device receives and plays the sounds.

The method according to any one of Embodiments 13-17, wherein the vagal nerve stimulation module comprises one or more sense stimulation instructions for touch, and the one or more sense stimulation instructions for touch include one or more instructions for abdominal breathing, controlling rate of breathing, cold massage, coughing, and skin massage.

The method according to any one of Embodiments 13-18, wherein the vagal nerve stimulation module comprises one or more sense stimulation instructions for touch, and the one or more sense stimulation instructions for touch include one or more instructions for abdominal breathing.

The method according to any one of Embodiments 13-19, wherein the vagal nerve stimulation module comprises one or more sense stimulation instructions for taste, and the one or more sense stimulation instructions for taste include one or more instructions to eat food to stimulate digestive glands in the subject.

The method according to Embodiment 20, wherein the electronic device receives and display information related to the food.

The method according to any one of Embodiments 13-21, wherein the vagal nerve stimulation module comprises one or more sense stimulation instructions for smell, and the one or more sense stimulation instructions for smell include one or more instructions to stimulate digestive glands and/or to relax.

The method according to Embodiment 22, wherein the electronic device is configured to release a scent for aroma therapy.

The method according to any one of Embodiments 1-23, wherein the one or more first modules comprise the aerobic exercise module, and the aerobic exercise module comprises one or more first instructions to increase adiponectin secretion in the subject.

The method according to Embodiment 24, wherein said one or more first instructions comprise one or more instructions for walking, biking, aerobic dance and/or swimming.

The method according to Embodiment 25, wherein the wherein the electronic device displays a guide screen indicating that the subject needs rest when maximum heart rate is reached.

The method according to Embodiment 26, wherein the maximum heart rate is calculated as [{207-(0.7*age)}-basal heart rate]*0.7+basal heart

The method according to Embodiment 27, wherein the basal heart rate is the subject's stable heart rate when setting up the device for the first time.

The method according to Embodiment 28, wherein when the device detects abnormal heart rate, a guide screen indicates that the subject should sufficiently rest.

The method according to Embodiment 29, wherein sufficient rest is about 10 minutes, or whenever the subject's heart rate falls below the maximum heart rate.

The method according to Embodiment 2, wherein the one or more first modules comprise the voluntary skeletal muscle exercise module, and the voluntary skeletal muscle exercise module compromise one or more first instructions for gravity exercise sessions, anti-gravity exercise sessions, lying down exercise sessions, and sitting exercise sessions.

The method according to Embodiment 31, wherein said one or more first instructions for gravity exercise sessions comprise one or more instructions for finger taps and heel taps.

The method according to Embodiment 31, wherein said one or more first instructions for anti-gravity exercise sessions comprise one or more instructions for head lifting, raising up hand, and raising up leg.

The method according to Embodiment 31, wherein said one or more first instructions for lying down exercise sessions comprise one or more instructions for hand gripping, fist clenching, and head turning.

The method according to Embodiment 31, wherein said one or more first instructions for sitting exercise sessions comprise one or more instructions for phone lifting, screen touching, raising up hands, raising up legs, hand gripping, and head turning.

The method according to Embodiment 2 or Embodiment 31, wherein voluntary skeletal muscle exercise module is scheduled for 45 days.

The method according to Embodiment 36, wherein the voluntary skeletal muscle exercise module is repeated and scheduled for a total of 90 days.

The method according to Embodiment 31 or 32, wherein one or more first instructions for gravity exercise sessions are scheduled for about 5 days.

The method according to Embodiment 31 or 33, wherein one or more first instructions for anti-gravity exercise sessions are scheduled for about 5 days

The method according to Embodiment 31 or 34, wherein one or more first instructions for lying down exercise sessions are scheduled for about 20 days.

The method according to Embodiment 31 or 35, wherein one or more first instructions for sitting down exercise sessions are scheduled for about 15 days.

The method according to Embodiment 31, wherein the one or more instructions for gravity exercise sessions, anti-gravity exercise sessions, lying down exercise sessions, and sitting exercise sessions are scheduled for about 20 minutes per session each day.

The method according to Embodiment 42, wherein the electronic device displays a guide screen indicating that the subject needs rest when maximum heart rate is reached.

The method according to Embodiment 43, wherein the maximum heart rate is calculated as [{207-(0.7*age)}-basal heart rate]*0.7+basal heart rate.

The method according to Embodiment 44, wherein the basal heart rate is the subject's stable heart rate when setting up the device for the first time.

The method according to Embodiment 45, wherein when the device detects abnormal heart rate, a guide screen indicates that the subject should sufficiently rest.

The method according to Embodiment 46, wherein sufficient rest is about 10 minutes, or whenever the subject's heart rate falls below the maximum heart rate.

The method according to any one of Embodiments 1-47, wherein the one or more first modules comprise the relaxation module, and the relaxation module comprises one or more first instructions to increase adiponectin secretion in the subject.

The method according to Embodiment 48, wherein the one or more first instructions comprise one or more breathing instructions.

The method according to Embodiment 48 or 49, wherein said one or more first instructions comprise one or more sound instructions to hear relaxing sound.

The method according to Embodiment 50, wherein the device receives and plays the relaxing sound.

The method according to Embodiment 48, wherein said one or more first instructions comprise one or more meditation instructions.

The method according to Embodiment 48, wherein said one or more first instructions comprise one or more massage instructions.

The method according to any one of Embodiments 48-53, wherein said instructions are scheduled for about 5 minutes.

The method according to any one of Embodiments 1-54, wherein the subject is an early cancer patient.

The method according to any one of Embodiments 1-54, wherein the subject has a cancer mass having a diameter of 3 cm or less.

The method according to any one of Embodiments 1 and 12-54, wherein the subject is a late cancer patient, and the method excludes providing a voluntary skeletal muscle exercise module.

The method according to any one of Embodiments 1 and 12-54, wherein the subject has a cancer mass having a diameter of more than 3 cm, and the method excludes providing a voluntary skeletal muscle exercise module.

The method according to Embodiment 55 or 56, wherein the subject has moderate muscle atrophy, and the one or more first modules consists of the voluntary skeletal muscle exercise module, the vagal nerve stimulation module, the aerobic exercise module, and the relaxation module.

The method according to Embodiment 58 or 59, wherein the subject has moderate muscle atrophy, and the one or more first modules consists of the vagal nerve stimulation module, the aerobic exercise module, and the relaxation module.

The method according to any one of Embodiments 1-60, wherein the subject has moderate muscle atrophy (in spec, define the moderate muscle atrophy as still being able to walk).

The method according to any one of Embodiments 1, 12-48, and 48-58, wherein the subject has severe muscle atrophy, and the method excludes providing a voluntary skeletal muscle exercise module and further excludes providing an aerobic exercise module.

The method according to Embodiment 62, wherein the one or more first modules consists of the vagal nerve stimulation module and the relaxation module.

The method according to any according to any one of Embodiments 1-63, wherein the server receives the one or more second instructions from an external reviewer.

The method according to any one of Embodiments 64, wherein the external reviewer comprises a health professional.

The method according to any one of Embodiments 64, wherein the external reviewer comprises an artificial intelligence (AI).

The method according to any one of Embodiments 1-66, wherein the sensor comprises one or more of: a camera, an accelerometer, a magnetometer, a light sensor, a microphone, a proximity sensor, a touch sensor, a gyroscope, a Global Positioning System (GPS) sensor, an ambient light sensor, a fingerprint sensor, a pedometer, a heart rate sensor, and a thermometer.

The method according to any one of Embodiments 1-67, wherein the sensor comprises a touch sensor, and the subject provides the adherence information to the electronic device using the touch sensor.

A system for treating cancer cachexia in a subject, comprising: a digital apparatus configured to execute a digital application comprising one or more first modules, for treating cancer cachexia in a subject, wherein the digital apparatus comprises a sensor for sensing adherence by the subject to a first set of instructions of the one or more first modules; a healthcare provider portal configured to provide one or more options to a healthcare provider to perform one or more tasks to prescribe treatment for the cancer cachexia in the subject based on information received from the digital application; and an administrative portal configured to provide one or more options to an administrator of the system to perform one or more tasks to manage access to the system by the healthcare provider.

The system of Embodiment 69, wherein the digital application for treating cancer cachexia instructs a processor of the digital apparatus to execute operations comprising:

generating digital therapeutic modules for treating cancer cachexia based on a mechanism of action in and a therapeutic hypothesis for the cancer cachexia.

The system of Embodiment 70, wherein the generating of the digital therapeutic modules comprises generating the digital therapeutic modules based on biochemical factors related to the cancer cachexia.

The system according to Embodiment 69, wherein the one or more first modules comprise the voluntary skeletal muscle exercise module, and the voluntary skeletal muscle exercise module comprises one or more first instructions to increase MGF/IGF-1 secretion in the subject.

The system according to Embodiment 72, wherein the voluntary skeletal muscle exercise module comprises a screen touching exercise instruction to touch a randomly generated displayed animation on the electronic device.

The system according to Embodiment 72, wherein the voluntary skeletal muscle exercise module comprises a head lifting exercise instruction to lift and position a head of the subject at a predetermined angle from the reference plane.

The system according to Embodiment 72, wherein the voluntary skeletal muscle exercise module comprises a head turning exercise instruction to turn a head of the subject clockwise and/or counterclockwise at a predetermined angle from the reference plane.

The system according to Embodiment 72, wherein the voluntary skeletal muscle exercise module comprises a hand gripping exercise instruction to grip a fist of the subject at a rate matching a displayed animation on the electronic device.

The system according to Embodiment 72, wherein the voluntary skeletal muscle exercise module comprises an arm shaking exercise instruction to move an object displayed on the electronic device across the screen with an index finger of the subject.

The system according to Embodiment 72, wherein the voluntary skeletal muscle exercise module comprises a leg lifting exercise instruct to lift their legs as measured by a change in a background area captured by the electronic device

The system according to Embodiment 72, wherein said one or more first instructions comprise instructions for aerobic, resistance, and/or concurrent exercise.

The system according to any one of Embodiments 69-79, wherein the subject inputs health information into the electronic device.

The system according to Embodiment 80, wherein the health information comprises of the subject's sleep schedule, blood sugar levels, protein, amino acid, anti-inflammatory health food intake information, appetite stimulant consumption information, and albumin levels.

The system according to any one of Embodiments 69-81, wherein the one or more first modules comprise the vagal nerve stimulation module, and the vagal nerve stimulation module comprises one or more first instructions to reduce inflammation in the subject.

The system according to any one of Embodiments 69-82, wherein the one or more first modules comprise the vagal nerve stimulation module, and the vagal nerve stimulation module comprises at least one instruction selected from the group consisting of sense stimulation instructions for sight, sound, touch, taste, and smell.

The system according to Embodiment 83, wherein the vagal nerve stimulation module comprises one or more sense stimulation instructions for sight, and the one or more sense stimulation instructions for sight include one or more instructions to view one or more figures (define in spec to include specific photos) to stimulate autonomic nervous system.

The system according to Embodiment 84, wherein the electronic device receives and displays the figures.

The system according to any one of Embodiments 83-85, wherein the vagal nerve stimulation module comprises one or more sense stimulation instructions for sound, and the one or more sense stimulation instructions for sound include one or more instructions to hear one or more sounds to cause horror or relaxation.

The system according to Embodiment 86, wherein the electronic device receives and plays the sounds.

The system according to any one of Embodiments 83-87, wherein the vagal nerve stimulation module comprises one or more sense stimulation instructions for touch, and the one or more sense stimulation instructions for touch include one or more instructions for abdominal breathing, controlling rate of breathing, cold massage, coughing, and skin massage.

The system according to any one of Embodiments 83-88, wherein the vagal nerve stimulation module comprises one or more sense stimulation instructions for touch, and the one or more sense stimulation instructions for touch include one or more instructions for abdominal breathing.

The system according to any one of Embodiments 83-89, wherein the vagal nerve stimulation module comprises one or more sense stimulation instructions for taste, and the one or more sense stimulation instructions for taste include one or more instructions to eat food to stimulate digestive glands in the subject.

The system according to Embodiment 90, wherein the electronic device receives and display information related to the food.

The system according to any one of Embodiments 83-91, wherein the vagal nerve stimulation module comprises one or more sense stimulation instructions for smell, and the one or more sense stimulation instructions for smell include one or more instructions to stimulate digestive glands and/or to relax.

The system according to Embodiment 92, wherein the electronic device is configured to release a scent for aroma therapy.

The system according to any one of Embodiments 69-93, wherein the one or more first modules comprise the aerobic exercise module, and the aerobic exercise module comprises one or more first instructions to increase adiponectin secretion in the subject.

The system according to Embodiment 94, wherein said one or more first instructions comprise one or more instructions for walking, biking, aerobic dance and/or swimming.

The system according to Embodiment 95, wherein the wherein the electronic device displays a guide screen indicating that the subject needs rest when maximum heart rate is reached.

The system according to Embodiment 96, wherein the maximum heart rate is calculated as [{207-(0.7*age)}-basal heart rate]*0.7+basal heart

The system according to Embodiment 97, wherein the basal heart rate is the subject's stable heart rate when setting up the device for the first time.

The system according to Embodiment 98, wherein when the device detects abnormal heart rate, a guide screen indicates that the subject should sufficiently rest.

The system according to Embodiment 99, wherein sufficient rest is about 10 minutes, or whenever the subject's heart rate falls below the maximum heart rate.

The system according to Embodiment 72, wherein the one or more first modules comprise the voluntary skeletal muscle exercise module, and the voluntary skeletal muscle exercise module compromise one or more first instructions for gravity exercise sessions, anti-gravity exercise sessions, lying down exercise sessions, and sitting exercise sessions.

The system according to Embodiment 101, wherein said one or more first instructions for gravity exercise sessions comprise one or more instructions for finger taps and heel taps.

The system according to Embodiment 101, wherein said one or more first instructions for anti-gravity exercise sessions comprise one or more instructions for head lifting, raising up hand, and raising up leg.

The system according to Embodiment 101, wherein said one or more first instructions for lying down exercise sessions comprise one or more instructions for hand gripping, fist clenching, and head turning.

The system according to Embodiment 101, wherein said one or more first instructions for sitting exercise sessions comprise one or more instructions for phone lifting, screen touching, raising up hands, raising up legs, hand gripping, and head turning.

The system according to Embodiment 72 or Embodiment 101, wherein voluntary skeletal muscle exercise module is scheduled for 45 days.

The system according to Embodiment 106, wherein the voluntary skeletal muscle exercise module is repeated and scheduled for a total of 90 days.

The system according to Embodiment 101 or 102, wherein one or more first instructions for gravity exercise sessions are scheduled for about 5 days.

The system according to Embodiment 101 or 103, wherein one or more first instructions for anti-gravity exercise sessions are scheduled for about 5 days

The system according to Embodiment 101 or 104, wherein one or more first instructions for lying down exercise sessions are scheduled for about 20 days.

The system according to Embodiment 101 or 105, wherein one or more first instructions for sitting down exercise sessions are scheduled for about 15 days.

The system according to Embodiment 101, wherein the one or more instructions for gravity exercise sessions, anti-gravity exercise sessions, lying down exercise sessions, and sitting exercise sessions are scheduled for about 20 minutes per session each day.

The system according to Embodiment 112, wherein the electronic device displays a guide screen indicating that the subject needs rest when maximum heart rate is reached.

The system according to Embodiment 113, wherein the maximum heart rate is calculated as [{207−(0.7*age)}-basal heart rate]*0.7+basal heart rate.

The system according to Embodiment 114, wherein the basal heart rate is the subject's stable heart rate when setting up the device for the first time.

The system according to Embodiment 115, wherein when the device detects abnormal heart rate, a guide screen indicates that the subject should sufficiently rest.

The system according to Embodiment 116, wherein sufficient rest is about 10 minutes, or whenever the subject's heart rate falls below the maximum heart rate.

The system according to any one of Embodiments 69-117, wherein the one or more first modules comprise the relaxation module, and the relaxation module comprises one or more first instructions to increase adiponectin secretion in the subject.

The system according to Embodiment 118, wherein the one or more first instructions comprise one or more breathing instructions.

The system according to Embodiments 118 or 119, wherein said one or more first instructions comprise one or more sound instructions to hear relaxing sound.

The system according to Embodiment 120, wherein the device receives and plays the relaxing sound.

The system according to Embodiment 118, wherein said one or more first instructions comprise one or more meditation instructions.

The system according to Embodiment 118, wherein said one or more first instructions comprise one or more massage instructions.

The system according to any one of Embodiments 118-124, wherein said instructions are scheduled for about 5 minutes.

The system according to any one of Embodiments 69-124, wherein the subject is an early cancer patient.

The system according to any one of Embodiments 69-124, wherein the subject has a cancer mass having a diameter of 3 cm or less.

The system according to any one of Embodiments 69 and 82-124, wherein the subject is a late cancer patient, and the system excludes providing a voluntary skeletal muscle exercise module.

The system according to any one of Embodiments 69 and 82-124, wherein the subject has a cancer mass having a diameter of more than 3 cm, and the system excludes providing a voluntary skeletal muscle exercise module.

The system according to Embodiment 125 or 126, wherein the subject has moderate muscle atrophy, and the one or more first modules consists of the voluntary skeletal muscle exercise module, the vagal nerve stimulation module, the aerobic exercise module, and the relaxation module.

The system according to Embodiment 128 or 129, wherein the subject has moderate muscle atrophy, and the one or more first modules consists of the vagal nerve stimulation module, the aerobic exercise module, and the relaxation module.

The system according to any one of Embodiments 69-130, wherein the subject has moderate muscle atrophy (in spec, define the moderate muscle atrophy as still being able to walk).

The system according to any one of Embodiments 69, 82-118, and 118-128, wherein the subject has severe muscle atrophy (in spec, define the severe muscle atrophy as not being able to walk), and the system excludes providing a voluntary skeletal muscle exercise module and further excludes providing an aerobic exercise module.

The system according to Embodiment 132 wherein the one or more first modules consists of the vagal nerve stimulation module and the relaxation module.

The system according to any one of Embodiments 69-133, wherein the digital application transmits data to a server, and wherein the server receives the one or more second instructions from an external reviewer.

The system according to any one of Embodiments 69-134, wherein the external reviewer comprises a health professional.

The system according to any one of Embodiments 69-134, wherein the external reviewer comprises an artificial intelligence (AI).

The system according to any one of Embodiments 69-136, wherein the sensor comprises one or more of: a camera, an accelerometer, a magnetometer, a light sensor, a microphone, a proximity sensor, a touch sensor, a gyroscope, a Global Positioning System (GPS) sensor, an ambient light sensor, a fingerprint sensor, a pedometer, a heart rate sensor, and a thermometer.

The system according to any one of Embodiments 69-137, wherein the sensor comprises a touch sensor, and the subject provides the adherence information to the electronic device using the touch sensor.

The system according to any one of Embodiment 69-138, wherein the one or more options provided to the healthcare provider are selected from the group consisting of adding or removing the subject, viewing or editing personal information for the subject, viewing adherence information for the subject, viewing a result of the subject for one or more at least partially completed digital therapeutic modules, prescribing one or more digital therapeutic modules to the subject, altering a prescription for one or more digital therapeutic modules, and communicating with the subject.

The system of Embodiment 139, wherein the one or more options comprise the viewing or editing personal information for the subject, and the personal information comprises one or more selected from the group consisting of an identification number for the subject, a name of the subject, a date of birth of the subject, an email of the subject, an email of the guardian of the subject, a contact phone number for the subject, a prescription for the subject, and one or more notes made by the healthcare provider about the subject.

The system of Embodiment 140, wherein the personal information comprises the prescription for the subject, and the prescription for the subject comprises one or more selected from the group consisting of a prescription identification number, a prescription type, a start date, a duration, a completion date, a number of scheduled or prescribed digital therapeutic modules to be performed by the subject, and a number of scheduled or prescribed digital therapeutic modules to be performed by the subject per day.

The system of any one of Embodiments 139-141, wherein the one or more options comprise the viewing the adherence information, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules.

The system of any one of Embodiments 139-143, wherein the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, an indicator of whether the scheduled or prescribed digital therapeutic module was fully or partially completed, and an exercise intensity (EI).

The system of Embodiment 69-143, wherein the one or more options provided to the administrator of the system are selected from the group consisting of adding or removing the healthcare provider, viewing or editing personal information for the healthcare provider, viewing or editing de-identified information of the subject, viewing adherence information for the subject, viewing a result of the subject for one or more at least partially completed digital therapeutic modules, and communicating with the healthcare provider.

The system of Embodiment 144, wherein the one or more options comprise the viewing or editing the personal information, and the personal information of the healthcare provider comprises one or more selected from the group consisting of an identification number for the healthcare provider, a name of the healthcare provider, an email of the healthcare provider, and a contact phone number for the healthcare provider.

The system of Embodiment 144 or 145, wherein the one or more options comprise the viewing or editing the de-identified information of the subject, and the de-identified information of the subject comprises one or more selected from the group consisting of an identification number for the subject, and the healthcare provider for the subject.

The system of any one of Embodiments 144-146, wherein the one or more options comprise the viewing the adherence information for the subject, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules.

The system of any one of Embodiments 139-147, wherein the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, an indicator of whether the scheduled or prescribed digital therapeutic module was fully or partially completed, and an exercise intensity (EI).

The system of Embodiment 69-148, wherein the digital application further comprises a push alarm for one or more of reminding the subject complete a digital therapeutic module.

The system of Embodiment 69-149, wherein the digital apparatus comprises: a digital instruction generation unit configured to generate digital therapeutic modules for treating cancer cachexia, generate digital instructions based on the digital therapeutic modules, and provide the digital instructions to the subject; and an outcome collection unit configured to collect the subject's execution outcomes of the digital instructions.

The system of Embodiment 69-150, wherein the digital instruction generation unit generates the digital therapeutic modules based on biochemical factors related to the cancer cachexia onset.

The system of Embodiment 151, wherein the biochemical factors comprise insulin-like growth factor 1 (IGF1) and hypoxia-inducible factor 1 (HIF1).

The system of Embodiment 69-152, wherein the digital instruction generation unit generates the digital therapeutic modules based on the inputs from the healthcare provider.

The system of Embodiment 69-153, wherein the digital instruction generation unit generates the digital therapeutic modules based on information received from the subject.

The system of Embodiment 154, wherein the information is received from the subject comprises at least one of basal factors, medical information, and digital therapeutics literacy of the subject, the basal factors including the subject's activity, heart rate, sleep, and diet (including nutrition and calories), the medical information including the subject's electronic medical record (EMR), family history, genetic vulnerability, and genetic susceptibility, and the digital therapeutics literacy including the subject's accessibility, and technology adoption to the digital therapeutics and the apparatus.

The system of Embodiment 69-155, wherein the digital instruction generation unit generates the digital therapeutic modules matching to imaginary parameters which correspond to the mechanism of action in and the therapeutic hypothesis for the cancer cachexia.

The system of Embodiment 156, wherein the imaginary parameters are deduced in relation to the subject's environment, behaviors, emotions, and cognition.

The system of Embodiment 69-157, wherein the outcome collection unit collects the execution outcomes of the digital instructions by monitoring the subject's adherence to the digital instructions or allowing the subject to directly input the subject's adherence to the digital instructions.

The system of Embodiment 69-158, wherein the generation of the digital instructions at the digital instruction generation unit and the collection of the subject's execution outcomes of the digital instructions at the outcome collection unit are repeatedly executed several times with multiple feedback loops, and the digital instruction generation unit generates the subject's digital instructions for this cycle based on the subject's digital instructions in the previous cycle and the execution outcome data on the subject's digital instructions in the previous cycle collected at the outcome collection unit.

A computing system for treating cancer cachexia in a subject in need thereof, comprising: a display configured to provide, to the subject, one or more first modules selected from the group consisting of a voluntary skeletal muscle exercise module, a vagal nerve stimulation module, an aerobic exercise module, and a relaxation module, each of the one or more first modules comprising one or more first instructions for the subject to follow; a sensor configured to sense adherence by the subject to the instructions of the one or more first modules; a transmitter configured to transmit adherence information, based on the adherence, to a server; and a receiver configured to receive, from the server, one or more second instructions based on the adherence information, wherein the display is further configured to provide, to the subject, one or more second modules selected from the group consisting of a voluntary skeletal muscle exercise module, a vagal nerve stimulation module, an aerobic exercise module, and a relaxation module, each of the one or more second modules comprising the one or more second instructions.

The computing system of Embodiment 160, wherein the digital application for treating cancer cachexia instructs a processor of the digital apparatus to execute operations comprising: generating digital therapeutic modules for treating cancer cachexia based on a mechanism of action in and a therapeutic hypothesis for the cancer cachexia.

The computing system of Embodiment 161, wherein the generating of the digital therapeutic modules comprises generating the digital therapeutic modules based on biochemical factors related to the cancer cachexia.

The computing system of Embodiment 162, wherein the one or more first modules comprise the voluntary skeletal muscle exercise module, and the voluntary skeletal muscle exercise module comprises one or more first instructions to increase MGF/IGF-1 secretion in the subject.

The computing system of Embodiment 163, wherein the voluntary skeletal muscle exercise module comprises a screen touching exercise instruction to touch a randomly generated displayed animation on the electronic device.

The computing system of Embodiment 163, wherein the voluntary skeletal muscle exercise module comprises a head lifting exercise instruction to lift and position a head of the subject at a predetermined angle from the reference plane.

The computing system of Embodiment 163, wherein the voluntary skeletal muscle exercise module comprises a head turning exercise instruction to turn a head of the subject clockwise and/or counterclockwise at a predetermined angle from the reference plane.

The computing system of Embodiment 163, wherein the voluntary skeletal muscle exercise module comprises a hand gripping exercise instruction to grip a fist of the subject at a rate matching a displayed animation on the electronic device.

The computing system of Embodiment 163, wherein the voluntary skeletal muscle exercise module comprises an arm shaking exercise instruction to move an object displayed on the electronic device across the screen with an index finger of the subject.

The computing system of Embodiment 163, wherein the voluntary skeletal muscle exercise module comprises a leg lifting exercise instruct to lift their legs as measured by a change in a background area captured by the electronic device.

The computing system of Embodiment 163, wherein said one or more first instructions comprise instructions for aerobic, resistance, and/or concurrent exercise.

The computing system of any one of Embodiments 160-170, wherein the subject inputs health information into the electronic device.

The computing system of Embodiment 171, wherein the health information comprises of the subject's sleep schedule, blood sugar levels, protein, amino acid, anti-inflammatory health food intake information, appetite stimulant consumption information, and albumin levels.

The computing system of any one of Embodiments 160-172, wherein the one or more first modules comprise the vagal nerve stimulation module, and the vagal nerve stimulation module comprises one or more first instructions to reduce inflammation in the subject.

The computing system of any one of Embodiments 160-173, wherein the one or more first modules comprise the vagal nerve stimulation module, and the vagal nerve stimulation module comprises at least one instruction selected from the group consisting of sense stimulation instructions for sight, sound, touch, taste, and smell.

The computing system of Embodiment 174, wherein the vagal nerve stimulation module comprises one or more sense stimulation instructions for sight, and the one or more sense stimulation instructions for sight include one or more instructions to view one or more figures (define in spec to include specific photos) to stimulate autonomic nervous system.

The computing system of Embodiment 175, wherein the electronic device receives and displays the figures.

The computing system of any one of Embodiments 173-175, wherein the vagal nerve stimulation module comprises one or more sense stimulation instructions for sound, and the one or more sense stimulation instructions for sound include one or more instructions to hear one or more sounds to cause horror or relaxation.

The computing system of Embodiment 177, wherein the electronic device receives and plays the sounds.

The computing system of any one of Embodiments 173-178, wherein the vagal nerve stimulation module comprises one or more sense stimulation instructions for touch, and the one or more sense stimulation instructions for touch include one or more instructions for abdominal breathing, controlling rate of breathing, cold massage, coughing, and skin massage.

The computing system of any one of Embodiments 173-179, wherein the vagal nerve stimulation module comprises one or more sense stimulation instructions for touch, and the one or more sense stimulation instructions for touch include one or more instructions for abdominal breathing.

The computing system of any one of Embodiments 173-180, wherein the vagal nerve stimulation module comprises one or more sense stimulation instructions for taste, and the one or more sense stimulation instructions for taste include one or more instructions to eat food to stimulate digestive glands in the subject.

The computing system of Embodiment 181, wherein the electronic device receives and display information related to the food. The computing system of any one of Embodiments 173-182, wherein the vagal nerve stimulation module comprises one or more sense stimulation instructions for smell, and the one or more sense stimulation instructions for smell include one or more instructions to stimulate digestive glands and/or to relax.

The computing system of Embodiment 183, wherein the electronic device is configured to release a scent for aroma therapy.

The computing system of any one of Embodiments 160-184, wherein the one or more first modules comprise the aerobic exercise module, and the aerobic exercise module comprises one or more first instructions to increase adiponectin secretion in the subject.

The computing system of Embodiment 185, wherein said one or more first instructions comprise one or more instructions for walking, biking, aerobic dance and/or swimming.

The computing system of Embodiment 186, wherein the wherein the electronic device displays a guide screen indicating that the subject needs rest when maximum heart rate is reached.

The computing system of Embodiment 187, wherein the maximum heart rate is calculated as [{207−(0.7*age)}-basal heart rate]*0.7+basal heart

The computing system of Embodiment 188, wherein the basal heart rate is the subject's stable heart rate when setting up the device for the first time.

The computing system of Embodiment 189, wherein when the device detects abnormal heart rate, a guide screen indicates that the subject should sufficiently rest.

The computing system of Embodiment 190, wherein sufficient rest is about 10 minutes, or whenever the subject's heart rate falls below the maximum heart rate.

The computing system of Embodiment 163, wherein the one or more first modules comprise the voluntary skeletal muscle exercise module, and the voluntary skeletal muscle exercise module compromise one or more first instructions for gravity exercise sessions, anti-gravity exercise sessions, lying down exercise sessions, and sitting exercise sessions.

The computing system of Embodiment 192, wherein said one or more first instructions for gravity exercise sessions comprise one or more instructions for finger taps and heel taps.

The computing system of Embodiment 192, wherein said one or more first instructions for anti-gravity exercise sessions comprise one or more instructions for head lifting, raising up hand, and raising up leg.

The computing system of Embodiment 192, wherein said one or more first instructions for lying down exercise sessions comprise one or more instructions for hand gripping, fist clenching, and head turning.

The computing system of Embodiment 192, wherein said one or more first instructions for sitting exercise sessions comprise one or more instructions for phone lifting, screen touching, raising up hands, raising up legs, hand gripping, and head turning.

The computing system of Embodiment 163 or Embodiment 192, wherein voluntary skeletal muscle exercise module is scheduled for 45 days.

The computing system of Embodiment 195, wherein the voluntary skeletal muscle exercise module is repeated and scheduled for a total of 90 days.

The computing system of Embodiment 192 or 193, wherein one or more first instructions for gravity exercise sessions are scheduled for about 5 days.

The computing system of Embodiment 192 or 194, wherein one or more first instructions for anti-gravity exercise sessions are scheduled for about 5 days

The computing system of Embodiment 192 or 195, wherein one or more first instructions for lying down exercise sessions are scheduled for about 20 days.

The computing system of Embodiment 192 or 196, wherein one or more first instructions for sitting down exercise sessions are scheduled for about 15 days.

The computing system of Embodiment 192, wherein the one or more instructions for gravity exercise sessions, anti-gravity exercise sessions, lying down exercise sessions, and sitting exercise sessions are scheduled for about 20 minutes per session each day.

The computing system of Embodiment 203, wherein the electronic device displays a guide screen indicating that the subject needs rest when maximum heart rate is reached.

The computing system of Embodiment 204, wherein the maximum heart rate may be calculated as [{207−(0.7*age)}-basal heart rate]*0.7+basal heart rate.

The computing system of Embodiment 205, wherein the basal heart rate is the subject's stable heart rate when setting up the device for the first time.

The computing system of Embodiment 206, wherein when the device detects abnormal heart rate, a guide screen indicates that the subject should sufficiently rest.

The computing system of Embodiment 207, wherein sufficient rest is about 10 minutes, or whenever the subject's heart rate falls below the maximum heart rate.

The computing system of any one of Embodiments 160-208, wherein the one or more first modules comprise the relaxation module, and the relaxation module comprises one or more first instructions to increase adiponectin secretion in the subject.

The computing system of Embodiment 209, wherein the one or more first instructions comprise one or more breathing instructions.

The computing system of Embodiment 209 or 210, wherein said one or more first instructions comprise one or more sound instructions to hear relaxing sound.

The computing system of Embodiment 211, wherein the device receives and plays the relaxing sound.

The computing system of Embodiment 209, wherein said one or more first instructions comprise one or more meditation instructions.

The computing system of Embodiment 209, wherein said one or more first instructions comprise one or more massage instructions.

The computing system of any one of Embodiments 209-214, wherein said instructions are scheduled for about 5 minutes.

The computing system of any one of Embodiments 160-214, wherein the subject is an early cancer patient.

The computing system of any one of Embodiments 160-214, wherein the subject has a cancer mass having a diameter of 3 cm or less.

The computing system of any one of Embodiments 160 and 173-215, wherein the subject is a late cancer patient, and the system excludes providing a voluntary skeletal muscle exercise module.

The computing system of any one of Embodiments 160 and 173-215, wherein the subject has a cancer mass having a diameter of more than 3 cm, and the system excludes providing a voluntary skeletal muscle exercise module.

The computing system of Embodiment 216 or 217, wherein the subject has moderate muscle atrophy, and the one or more first modules consists of the voluntary skeletal muscle exercise module, the vagal nerve stimulation module, the aerobic exercise module, and the relaxation module.

The computing system of Embodiment 219 or 220, wherein the subject has moderate muscle atrophy, and the one or more first modules consists of the vagal nerve stimulation module, the aerobic exercise module, and the relaxation module.

The computing system of any one of Embodiments 160-221, wherein the subject has moderate muscle atrophy (in spec, define the moderate muscle atrophy as still being able to walk).

The computing system of any one of Embodiments 160, 173-209, and 209-219, wherein the subject has severe muscle atrophy (in spec, define the severe muscle atrophy as not being able to walk), and the system excludes providing a voluntary skeletal muscle exercise module and further excludes providing an aerobic exercise module.

The computing system of Embodiment 223, wherein the one or more first modules consists of the vagal nerve stimulation module and the relaxation module.

The computing system of any one of Embodiments 160-224, wherein the digital application transmits data to a server, and wherein the server receives the one or more second instructions from an external reviewer.

The computing system of any one of Embodiments 160-225, wherein the external reviewer comprises a health professional.

The computing system of any one of Embodiments 160-225, wherein the external reviewer comprises an artificial intelligence (AI).

The computing system of any one of Embodiments 160-227, wherein the sensor comprises one or more of: a camera, an accelerometer, a magnetometer, a light sensor, a microphone, a proximity sensor, a touch sensor, a gyroscope, a Global Positioning System (GPS) sensor, an ambient light sensor, a fingerprint sensor, a pedometer, a heart rate sensor, and a thermometer.

The computing system of any one of Embodiments 160-228, wherein the sensor comprises a touch sensor, and the subject provides the adherence information to the electronic device using the touch sensor.

The computing system of Embodiment 160-229, wherein the one or more options provided to the healthcare provider are selected from the group consisting of adding or removing the subject, viewing or editing personal information for the subject, viewing adherence information for the subject, viewing a result of the subject for one or more at least partially completed digital therapeutic modules, prescribing one or more digital therapeutic modules to the subject, altering a prescription for one or more digital therapeutic modules, and communicating with the subject.

The computing system of Embodiment 230, wherein the one or more options comprise the viewing or editing personal information for the subject, and the personal information comprises one or more selected from the group consisting of an identification number for the subject, a name of the subject, a date of birth of the subject, an email of the subject, an email of the guardian of the subject, a contact phone number for the subject, a prescription for the subject, and one or more notes made by the healthcare provider about the subject.

The computing system of Embodiment 231, wherein the personal information comprises the prescription for the subject, and the prescription for the subject comprises one or more selected from the group consisting of a prescription identification number, a prescription type, a start date, a duration, a completion date, a number of scheduled or prescribed digital therapeutic modules to be performed by the subject, and a number of scheduled or prescribed digital therapeutic modules to be performed by the subject per day.

The computing system of Embodiment 232, wherein the one or more options comprise the viewing the adherence information, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules.

The computing system of Embodiment 232, wherein the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, an indicator of whether the scheduled or prescribed digital therapeutic module was fully or partially completed, and an exercise intensity (EI).

The computing system of Embodiment 160-234, wherein the one or more options provided to the administrator of the system are selected from the group consisting of adding or removing the healthcare provider, viewing or editing personal information for the healthcare provider, viewing or editing de-identified information of the subject, viewing adherence information for the subject, viewing a result of the subject for one or more at least partially completed digital therapeutic modules, and communicating with the healthcare provider.

The computing system of Embodiment 235, wherein the one or more options comprise the viewing or editing the personal information, and the personal information of the healthcare provider comprises one or more selected from the group consisting of an identification number for the healthcare provider, a name of the healthcare provider, an email of the healthcare provider, and a contact phone number for the healthcare provider.

The computing system of Embodiment 235, wherein the one or more options comprise the viewing or editing the de-identified information of the subject, and the de-identified information of the subject comprises one or more selected from the group consisting of an identification number for the subject, and the healthcare provider for the subject.

The computing system of Embodiment 235, wherein the one or more options comprise the viewing the adherence information for the subject, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules.

The computing system of Embodiment 235, wherein the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, an indicator of whether the scheduled or prescribed digital therapeutic module was fully or partially completed, and an exercise intensity (EI).

The computing system of Embodiment 160-239, wherein the digital application further comprises a push alarm for one or more of reminding the subject complete a digital therapeutic module.

The computing system of Embodiment 160-240, wherein the digital apparatus comprises: a digital instruction generation unit configured to generate digital therapeutic modules for treating cancer cachexia, generate digital instructions based on the digital therapeutic modules, and provide the digital instructions to the subject; and an outcome collection unit configured to collect the subject's execution outcomes of the digital instructions.

The computing system of Embodiment 160-241, wherein the digital instruction generation unit generates the digital therapeutic modules based on biochemical factors related to the cancer cachexia onset.

The computing system of Embodiment 242, wherein the biochemical factors comprise insulin-like growth factor 1 (IGF1) and hypoxia-inducible factor 1 (HIF1).

The computing system of Embodiment 160-243, wherein the digital instruction generation unit generates the digital therapeutic modules based on the inputs from the healthcare provider.

The computing system of Embodiment 160-244, wherein the digital instruction generation unit generates the digital therapeutic modules based on information received from the subject.

The computing system of Embodiment 245, wherein the information is received from the subject comprises at least one of basal factors, medical information, and digital therapeutics literacy of the subject, the basal factors including the subject's activity, heart rate, sleep, and diet (including nutrition and calories), the medical information including the subject's electronic medical record (EMR), family history, genetic vulnerability, and genetic susceptibility, and the digital therapeutics literacy including the subject's accessibility, and technology adoption to the digital therapeutics and the apparatus.

The computing system of Embodiment 160-246, wherein the digital instruction generation unit generates the digital therapeutic modules matching to imaginary parameters which correspond to the mechanism of action in and the therapeutic hypothesis for the cancer cachexia.

The computing system of Embodiment 160, wherein the imaginary parameters are deduced in relation to the subject's environment, behaviors, emotions, and cognition.

The computing system of Embodiment 160-248, wherein the outcome collection unit collects the execution outcomes of the digital instructions by monitoring the subject's adherence to the digital instructions or allowing the subject to directly input the subject's adherence to the digital instructions.

The computing system of Embodiment 160-249, wherein the generation of the digital instructions at the digital instruction generation unit and the collection of the subject's execution outcomes of the digital instructions at the outcome collection unit are repeatedly executed several times with multiple feedback loops, and the digital instruction generation unit generates the subject's digital instructions for this cycle based on the subject's digital instructions in the previous cycle and the execution outcome data on the subject's digital instructions in the previous cycle collected at the outcome collection unit.

A non-transitory computer readable medium having stored thereon software instructions for treating cancer cachexia in a subject in need thereof that, when executed by a processor, cause the processor to: display, by an electronic device to the subject, one or more first modules selected from the group consisting of a voluntary skeletal muscle exercise module, a vagal nerve stimulation module, an aerobic exercise module, and a relaxation module, each of the one or more first modules comprising instructions for the subject to follow; sense, by a sensor in the electronic device, adherence by the subject to the instructions of the one or more first modules; transmit, by the electronic device, adherence information, based on the adherence, to a server; receive, from the server, one or more second instructions based on the adherence information; and display, to the subject, one or more second modules selected from the group consisting of a voluntary skeletal muscle exercise module, a vagal nerve stimulation module, an aerobic exercise module, and a relaxation module, the one or more second modules comprising the one or more second instructions.

The non-transitory computer readable medium of Embodiment 251, wherein the digital application for treating cancer cachexia instructs a processor of the digital apparatus to execute operations comprising: generating digital therapeutic modules for treating cancer cachexia based on a mechanism of action in and a therapeutic hypothesis for the cancer cachexia.

The non-transitory computer readable medium of Embodiment 252, wherein the generating of the digital therapeutic modules comprises generating the digital therapeutic modules based on biochemical factors related to the cancer cachexia.

The non-transitory computer readable medium of Embodiment 253, wherein the one or more first modules comprise the voluntary skeletal muscle exercise module, and the voluntary skeletal muscle exercise module comprises one or more first instructions to increase MGF/IGF-1 secretion in the subject.

The non-transitory computer readable medium of Embodiment 254, wherein the voluntary skeletal muscle exercise module comprises a screen touching exercise instruction to touch a randomly generated displayed animation on the electronic device.

The non-transitory computer readable medium of Embodiment 254, wherein the voluntary skeletal muscle exercise module comprises a head lifting exercise instruction to lift and position a head of the subject at a predetermined angle from the reference plane.

The non-transitory computer readable medium of Embodiment 254, wherein the voluntary skeletal muscle exercise module comprises a head turning exercise instruction to turn a head of the subject clockwise and/or counterclockwise at a predetermined angle from the reference plane.

The non-transitory computer readable medium of Embodiment 254, wherein the voluntary skeletal muscle exercise module comprises a hand gripping exercise instruction to grip a fist of the subject at a rate matching a displayed animation on the electronic device.

The non-transitory computer readable medium of Embodiment 254, wherein the voluntary skeletal muscle exercise module comprises an arm shaking exercise instruction to move an object displayed on the electronic device across the screen with an index finger of the subject.

The non-transitory computer readable medium of Embodiment 254, wherein the voluntary skeletal muscle exercise module comprises a leg lifting exercise instruct to lift their legs as measured by a change in a background area captured by the electronic device.

The non-transitory computer readable medium of Embodiment 254, wherein said one or more first instructions comprise instructions for aerobic, resistance, and/or concurrent exercise.

The non-transitory computer readable medium of any one of Embodiments 251-261, wherein the subject inputs health information into the electronic device.

The non-transitory computer readable medium of Embodiment 262, wherein the health information comprises of the subject's sleep schedule, blood sugar levels, protein, amino acid, anti-inflammatory health food intake information, appetite stimulant consumption information, and albumin levels.

The non-transitory computer readable medium of any one of Embodiments 251-263, wherein the one or more first modules comprise the vagal nerve stimulation module, and the vagal nerve stimulation module comprises one or more first instructions to reduce inflammation in the subject.

The non-transitory computer readable medium of any one of Embodiments 251-264, wherein the one or more first modules comprise the vagal nerve stimulation module, and the vagal nerve stimulation module comprises at least one instruction selected from the group consisting of sense stimulation instructions for sight, sound, touch, taste, and smell.

The non-transitory computer readable medium of Embodiment 265, wherein the vagal nerve stimulation module comprises one or more sense stimulation instructions for sight, and the one or more sense stimulation instructions for sight include one or more instructions to view one or more figures (define in spec to include specific photos) to stimulate autonomic nervous system.

The non-transitory computer readable medium of Embodiment 266, wherein the electronic device receives and displays the figures.

The non-transitory computer readable medium of any one of Embodiments 265-267, wherein the vagal nerve stimulation module comprises one or more sense stimulation instructions for sound, and the one or more sense stimulation instructions for sound include one or more instructions to hear one or more sounds to cause horror or relaxation.

The non-transitory computer readable medium of Embodiment 268, wherein the electronic device receives and plays the sounds.

The non-transitory computer readable medium of any one of Embodiments 265-269, wherein the vagal nerve stimulation module comprises one or more sense stimulation instructions for touch, and the one or more sense stimulation instructions for touch include one or more instructions for abdominal breathing, controlling rate of breathing, cold massage, coughing, and skin massage.

The non-transitory computer readable medium of any one of Embodiments 265-270, wherein the vagal nerve stimulation module comprises one or more sense stimulation instructions for touch, and the one or more sense stimulation instructions for touch include one or more instructions for abdominal breathing.

The non-transitory computer readable medium of any one of Embodiments 265-271, wherein the vagal nerve stimulation module comprises one or more sense stimulation instructions for taste, and the one or more sense stimulation instructions for taste include one or more instructions to eat food to stimulate digestive glands in the subject.

The non-transitory computer readable medium of Embodiment 272, wherein the electronic device receives and display information related to the food.

The non-transitory computer readable medium of any one of Embodiments 265-273, wherein the vagal nerve stimulation module comprises one or more sense stimulation instructions for smell, and the one or more sense stimulation instructions for smell include one or more instructions to stimulate digestive glands and/or to relax.

The non-transitory computer readable medium of Embodiment 274, wherein the electronic device is configured to release a scent for aroma therapy.

The non-transitory computer readable medium of any one of Embodiments 251-275, wherein the one or more first modules comprise the aerobic exercise module, and the aerobic exercise module comprises one or more first instructions to increase adiponectin secretion in the subject.

The non-transitory computer readable medium of Embodiment 276, wherein said one or more first instructions comprise one or more instructions for walking, biking, aerobic dance and/or swimming.

The non-transitory computer readable medium of Embodiment 277, wherein the wherein the electronic device displays a guide screen indicating that the subject needs rest when maximum heart rate is reached.

The non-transitory computer readable medium of Embodiment 278, wherein the maximum heart rate is calculated as [{207−(0.7*age)}-basal heart rate]*0.7+basal heart

The non-transitory computer readable medium of Embodiment 279, wherein the basal heart rate is the subject's stable heart rate when setting up the device for the first time.

The non-transitory computer readable medium of Embodiment 280, wherein when the device detects abnormal heart rate, a guide screen indicates that the subject should sufficiently rest.

The non-transitory computer readable medium of Embodiment 281, wherein sufficient rest is about 10 minutes, or whenever the subject's heart rate falls below the maximum heart rate.

The non-transitory computer readable medium of Embodiment 254, wherein the one or more first modules comprise the voluntary skeletal muscle exercise module, and the voluntary skeletal muscle exercise module compromise one or more first instructions for gravity exercise sessions, anti-gravity exercise sessions, lying down exercise sessions, and sitting exercise sessions.

The non-transitory computer readable medium of Embodiment 283, wherein said one or more first instructions for gravity exercise sessions comprise one or more instructions for finger taps and heel taps.

The non-transitory computer readable medium of Embodiment 283, wherein said one or more first instructions for anti-gravity exercise sessions comprise one or more instructions for head lifting, raising up hand, and raising up leg.

The non-transitory computer readable medium of Embodiment 283, wherein said one or more first instructions for lying down exercise sessions comprise one or more instructions for hand gripping, fist clenching, and head turning.

The non-transitory computer readable medium of Embodiment 283, wherein said one or more first instructions for sitting exercise sessions comprise one or more instructions for phone lifting, screen touching, raising up hands, raising up legs, hand gripping, and head turning.

The non-transitory computer readable medium of Embodiment 251 or Embodiment 283, wherein voluntary skeletal muscle exercise module is scheduled for 45 days.

The non-transitory computer readable medium of Embodiment 288, wherein the voluntary skeletal muscle exercise module is repeated and scheduled for a total of 90 days.

The non-transitory computer readable medium of Embodiment 283 or 284, wherein one or more first instructions for gravity exercise sessions are scheduled for about 5 days.

The non-transitory computer readable medium of Embodiment 283 or 285, wherein one or more first instructions for anti-gravity exercise sessions are scheduled for about 5 days

The non-transitory computer readable medium of Embodiment 283 or 286, wherein one or more first instructions for lying down exercise sessions are scheduled for about 20 days.

The non-transitory computer readable medium of Embodiment 283 or 287, wherein one or more first instructions for sitting down exercise sessions are scheduled for about 15 days.

The non-transitory computer readable medium of Embodiment 283, wherein the one or more instructions for gravity exercise sessions, anti-gravity exercise sessions, lying down exercise sessions, and sitting exercise sessions are scheduled for about 20 minutes per session each day.

The non-transitory computer readable medium of Embodiment 294, wherein the electronic device displays a guide screen indicating that the subject needs rest when maximum heart rate is reached.

The non-transitory computer readable medium of Embodiment 295, wherein the maximum heart rate is calculated as [{207−(0.7*age)}-basal heart rate]*0.7+basal heart rate.

The non-transitory computer readable medium of Embodiment 296, wherein the basal heart rate is the subject's stable heart rate when setting up the device for the first time.

The non-transitory computer readable medium of Embodiment 297, wherein when the device detects abnormal heart rate, a guide screen indicates that the subject should sufficiently rest.

The non-transitory computer readable medium of according to Embodiment 298, wherein sufficient rest is about 10 minutes, or whenever the subject's heart rate falls below the maximum heart rate.

The non-transitory computer readable medium of any one of Embodiments 251-299, wherein the one or more first modules comprise the relaxation module, and the relaxation module comprises one or more first instructions to increase adiponectin secretion in the subject.

The non-transitory computer readable medium of Embodiment 300, wherein the one or more first instructions comprise one or more breathing instructions.

The non-transitory computer readable medium of Embodiment 300 or 301, wherein said one or more first instructions comprise one or more sound instructions to hear relaxing sound.

The non-transitory computer readable medium of Embodiment 302, wherein the device receives and plays the relaxing sound.

The non-transitory computer readable medium of Embodiment 300, wherein said one or more first instructions comprise one or more meditation instructions.

The non-transitory computer readable medium of Embodiment 300, wherein said one or more first instructions comprise one or more massage instructions.

The non-transitory computer readable medium of any one of Embodiments 300-305, wherein said instructions are scheduled for about 5 minutes.

The non-transitory computer readable medium of any one of Embodiments 251-306, wherein the subject is an early cancer patient.

The non-transitory computer readable medium of any one of Embodiments 251-306, wherein the subject has a cancer mass having a diameter of 3 cm or less.

The non-transitory computer readable medium of any one of Embodiments 251 and 271-306, wherein the subject is a late cancer patient, and the system excludes providing a voluntary skeletal muscle exercise module.

The non-transitory computer readable medium of any one of Embodiments 251 and 271-306, wherein the subject has a cancer mass having a diameter of more than 3 cm, and the system excludes providing a voluntary skeletal muscle exercise module.

The non-transitory computer readable medium of Embodiment 307 or 308, wherein the subject has moderate muscle atrophy, and the one or more first modules consists of the voluntary skeletal muscle exercise module, the vagal nerve stimulation module, the aerobic exercise module, and the relaxation module.

The non-transitory computer readable medium of Embodiment 309 or 310, wherein the subject has moderate muscle atrophy, and the one or more first modules consists of the vagal nerve stimulation module, the aerobic exercise module, and the relaxation module.

The non-transitory computer readable medium of any one of Embodiments 251-312, wherein the subject has moderate muscle atrophy (in spec, define the moderate muscle atrophy as still being able to walk).

The non-transitory computer readable medium of any one of Embodiments 251, 271-300, and 300-310 wherein the subject has severe muscle atrophy (in spec, define the severe muscle atrophy as not being able to walk), and the system excludes providing a voluntary skeletal muscle exercise module and further excludes providing an aerobic exercise module.

The non-transitory computer readable medium of Embodiment 314, wherein the one or more first modules consists of the vagal nerve stimulation module and the relaxation module.

The non-transitory computer readable medium of any one of Embodiments 251-315, wherein the digital application transmits data to a server, and wherein the server receives the one or more second instructions from an external reviewer.

The non-transitory computer readable medium of any one of Embodiments 251-316, wherein the external reviewer comprises a health professional.

The non-transitory computer readable medium of any one of Embodiments 251-317, wherein the external reviewer comprises an artificial intelligence (AI).

The non-transitory computer readable medium of any one of Embodiments 251-318, wherein the sensor comprises one or more of: a camera, an accelerometer, a magnetometer, a light sensor, a microphone, a proximity sensor, a touch sensor, a gyroscope, a Global Positioning System (GPS) sensor, an ambient light sensor, a fingerprint sensor, a pedometer, a heart rate sensor, and a thermometer.

The non-transitory computer readable medium of any one of Embodiments 251-319, wherein the sensor comprises a touch sensor, and the subject provides the adherence information to the electronic device using the touch sensor.

The non-transitory computer readable medium of Embodiment 251-320, wherein the one or more options provided to the healthcare provider are selected from the group consisting of adding or removing the subject, viewing or editing personal information for the subject, viewing adherence information for the subject, viewing a result of the subject for one or more at least partially completed digital therapeutic modules, prescribing one or more digital therapeutic modules to the subject, altering a prescription for one or more digital therapeutic modules, and communicating with the subject.

The non-transitory computer readable medium of Embodiment 321, wherein the one or more options comprise the viewing or editing personal information for the subject, and the personal information comprises one or more selected from the group consisting of an identification number for the subject, a name of the subject, a date of birth of the subject, an email of the subject, an email of the guardian of the subject, a contact phone number for the subject, a prescription for the subject, and one or more notes made by the healthcare provider about the subject.

The non-transitory computer readable medium of Embodiment 322, wherein the personal information comprises the prescription for the subject, and the prescription for the subject comprises one or more selected from the group consisting of a prescription identification number, a prescription type, a start date, a duration, a completion date, a number of scheduled or prescribed digital therapeutic modules to be performed by the subject, and a number of scheduled or prescribed digital therapeutic modules to be performed by the subject per day.

The non-transitory computer readable medium of any one of Embodiments 321-323, wherein the one or more options comprise the viewing the adherence information, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules.

The non-transitory computer readable medium of any one of Embodiments 321-324, wherein the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, an indicator of whether the scheduled or prescribed digital therapeutic module was fully or partially completed, and an exercise intensity (EI).

The non-transitory computer readable medium of Embodiment 251-325, wherein the one or more options provided to the administrator of the system are selected from the group consisting of adding or removing the healthcare provider, viewing or editing personal information for the healthcare provider, viewing or editing de-identified information of the subject, viewing adherence information for the subject, viewing a result of the subject for one or more at least partially completed digital therapeutic modules, and communicating with the healthcare provider.

The non-transitory computer readable medium of Embodiment 326, wherein the one or more options comprise the viewing or editing the personal information, and the personal information of the healthcare provider comprises one or more selected from the group consisting of an identification number for the healthcare provider, a name of the healthcare provider, an email of the healthcare provider, and a contact phone number for the healthcare provider.

The non-transitory computer readable medium of Embodiment 326 or 327, wherein the one or more options comprise the viewing or editing the de-identified information of the subject, and the de-identified information of the subject comprises one or more selected from the group consisting of an identification number for the subject, and the healthcare provider for the subject.

The non-transitory computer readable medium of any one of Embodiments 326-328, wherein the one or more options comprise the viewing the adherence information for the subject, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules.

The non-transitory computer readable medium of any one of Embodiments 326-329, wherein the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, an indicator of whether the scheduled or prescribed digital therapeutic module was fully or partially completed, and an exercise intensity (EI).

The non-transitory computer readable medium of Embodiment 251-330, wherein the digital application further comprises a push alarm for one or more of reminding the subject complete a digital therapeutic module.

The non-transitory computer readable medium of Embodiment 251-331, wherein the digital apparatus comprises: a digital instruction generation unit configured to generate digital therapeutic modules for treating cancer cachexia, generate digital instructions based on the digital therapeutic modules, and provide the digital instructions to the subject; and an outcome collection unit configured to collect the subject's execution outcomes of the digital instructions.

The non-transitory computer readable medium of Embodiment 251-332, wherein the digital instruction generation unit generates the digital therapeutic modules based on biochemical factors related to the cancer cachexia onset.

The non-transitory computer readable medium of Embodiment 333, wherein the biochemical factors comprise insulin-like growth factor 1 (IGF1) and hypoxia-inducible factor 1 (HIF1).

The non-transitory computer readable medium of Embodiment 251-334, wherein the digital instruction generation unit generates the digital therapeutic modules based on the inputs from the healthcare provider.

The non-transitory computer readable medium of Embodiment 251-335, wherein the digital instruction generation unit generates the digital therapeutic modules based on information received from the subject.

The non-transitory computer readable medium of Embodiment 336, wherein the information is received from the subject comprises at least one of basal factors, medical information, and digital therapeutics literacy of the subject, the basal factors including the subject's activity, heart rate, sleep, and diet (including nutrition and calories), the medical information including the subject's electronic medical record (EMR), family history, genetic vulnerability, and genetic susceptibility, and the digital therapeutics literacy including the subject's accessibility, and technology adoption to the digital therapeutics and the apparatus.

The non-transitory computer readable medium of Embodiment 251-337, wherein the digital instruction generation unit generates the digital therapeutic modules matching to imaginary parameters which correspond to the mechanism of action in and the therapeutic hypothesis for the cancer cachexia.

The non-transitory computer readable medium of Embodiment 338, wherein the imaginary parameters are deduced in relation to the subject's environment, behaviors, emotions, and cognition.

The non-transitory computer readable medium of Embodiment 251-339, wherein the outcome collection unit collects the execution outcomes of the digital instructions by monitoring the subject's adherence to the digital instructions or allowing the subject to directly input the subject's adherence to the digital instructions.

The non-transitory computer readable medium of Embodiment 251-340, wherein the generation of the digital instructions at the digital instruction generation unit and the collection of the subject's execution outcomes of the digital instructions at the outcome collection unit are repeatedly executed several times with multiple feedback loops, and the digital instruction generation unit generates the subject's digital instructions for this cycle based on the subject's digital instructions in the previous cycle and the execution outcome data on the subject's digital instructions in the previous cycle collected at the outcome collection unit.

What is claimed is:

1. A method of treating cancer cachexia in a subject in need thereof, the method comprising:
providing, by an electronic device to the subject, one or more first modules selected from the group consisting of a voluntary skeletal muscle exercise module, a vagal nerve stimulation module, an aerobic exercise module, and a relaxation module, each of the one or more first modules comprising one or more first instructions for the subject to follow; and
providing, by the electronic device to the subject, one or more second modules selected from the group consisting of a voluntary skeletal muscle exercise module, a vagal nerve stimulation module, an aerobic exercise module, and a relaxation module, the one or more second modules comprising the one or more second instructions,
wherein the electronic device comprises a sensor sensing adherence by the subject to the first instructions of the one or more first modules, transmits adherence information, based on the adherence, to a server, and receives one or more second instructions from the server based on the adherence information.

2. The method according to claim 1, wherein the one or more first modules comprise the voluntary skeletal muscle exercise module, and the voluntary skeletal muscle exercise module comprises one or more instructions to increase MGF/IGF-1 secretion in the subject.

3. A computing system for treating cancer cachexia in a subject in need thereof, comprising:
a display configured to provide, to the subject, one or more first modules selected from the group consisting of a voluntary skeletal muscle exercise module, a vagal nerve stimulation module, an aerobic exercise module, and a relaxation module, each of the one or more first modules comprising one or more first instructions for the subject to follow;
a sensor configured to sense adherence by the subject to the instructions of the one or more first modules;
a transmitter configured to transmit adherence information, based on the adherence, to a server; and
a receiver configured to receive, from the server, one or more second instructions based on the adherence information,
wherein the display is further configured to provide, to the subject, one or more second modules selected from the group consisting of a voluntary skeletal muscle exercise module, a vagal nerve stimulation module, an aerobic exercise module, and a relaxation module, each of the one or more second modules comprising the one or more second instructions.

4. A non-transitory computer readable medium having stored thereon software instructions for treating cancer cachexia in a subject in need thereof that, when executed by a processor, cause the processor to:
display, by an electronic device to the subject, one or more first modules selected from the group consisting of a voluntary skeletal muscle exercise module, a vagal nerve stimulation module, an aerobic exercise module, and a relaxation module, each of the one or more first modules comprising instructions for the subject to follow;
sense, by a sensor in the electronic device, adherence by the subject to the instructions of the one or more first modules;
transmit, by the electronic device, adherence information, based on the adherence, to a server;
receive, from the server, one or more second instructions based on the adherence information; and
display, to the subject, one or more second modules selected from the group consisting of a voluntary skeletal muscle exercise module, a vagal nerve stimulation module, an aerobic exercise module, and a relaxation module, the one or more second modules comprising the one or more second instructions.
